(19) United States
(12) Patent Application Publication
(10) Pub. No.: US 2008/0275022 A1
(43) Pub. Date: Nov. 6, 2008

(54) Substituted Quinazolones as Anti-Cancer Agents

(75) Inventors: Brian Aquila, Marlborough, MA (US); Les Dakin, Natick, MA (US); Jayachandran Ezhuthachan, Brookline, MA (US); John Lee, Harvard, MA (US); Paul Lyne, Arlington, MA (US); Timothy Pontz, Cambridge, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 11/569,918

(22) PCT Filed: Jun. 14, 2005
(86) PCT No.: PCT/GB05/02327

§ 371 Date: Dec. 1, 2006

Related U.S. Application Data
(60) Provisional application No. 60/579,265, filed on Jun. 15, 2004.

(51) Int. Cl. A61K 31/517; C07D 239/88; C07D 413/02; A61K 31/5377; C07D 403/02; A61P 35/00

(52) U.S. Cl. 514/210.21; 544/287; 514/266.3; 514/234.5; 544/119

(57) ABSTRACT

The invention relates to chemical compounds of the formula (I): or pharmaceutically acceptable salts thereof, which possess B Raf inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

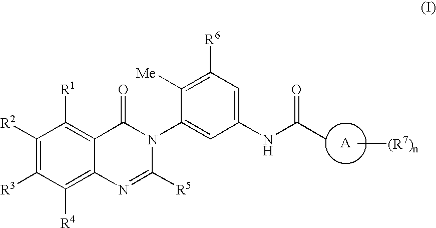

The invention relates to chemical compounds, or pharmaceutically acceptable salts thereof, which possess B-Raf inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The classical Ras, Raf, MAP protein kinase/extracellular signal-regulated kinase kinase (MEK), extracellular signal-regulated kinase (ERK) pathway plays a central role in the regulation of a variety of cellular functions dependent upon cellular context, including cellular proliferation, differentiation, survival, immortalization and angiogenesis (reviewed in Peyssonnaux and Eychene, Biology of the Cell, 2001, 93, 3-62). In this pathway, Raf family members are recruited to the plasma membrane upon binding to guanosine triphosphate (GTP) loaded Ras resulting in the phosphorylation and activation of Raf proteins. Activated Rafs then phosphorylate and activate MEKs, which in turn phosphorylate and activate ERKs. Upon activation, ERKs translocate from the cytoplasm to the nucleus resulting in the phosphorylation and regulation of activity of transcription factors such as Elk-1 and Myc.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp. Rev. Mol. Med., 2002, 25 April, http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway.

Three Raf serine/threonine protein kinase isoforms have been reported Raf-1/c-Raf, B-Raf and A-Raf (reviewed in Mercer and Pritchard, Biochim. Biophys. Acta, 2003, 1653, 25-40), the genes for which are thought to have arisen from gene duplication. All three Raf genes are expressed in most tissues with high-level expression of B-Raf in neuronal tissue and A-Raf in urogenital tissue. The highly homologous Raf family members have overlapping but distinct biochemical activities and biological functions (Hagemann and Rapp, Expt. Cell Res. 1999, 253, 34-46). Expression of all three Raf genes is required for normal murine development however both c-Raf and B-Raf are required to complete gestation. B-Raf −/− mice die at E12.5 due to vascular haemorrhaging caused by increased apoptosis of endothelial cells (Wojnowski et al., Nature Genet., 1997, 16, 293-297). B-Raf is reportedly the major isoform involved in cell proliferation and the primary target of oncogenic Ras. Activating somatic missense mutations have been identified exclusively for B-Raf, occurring with a frequency of 66% in malignant cutaneous melanomas (Davies et al., Nature, 2002, 417, 949-954) and also present in a wide range of human cancers, including but not limited to papillary thyroid tumours (Cohen et al., J. Natl. Cancer Inst., 2003, 95, 625-627), cholangiocarcinomas (Tannapfel et al., Gut, 2003, 52, 706-712), colon and ovarian cancers (Davies et al., Nature, 2002, 417, 949-954). The most frequent mutation in B-Raf (80%) is a glutamic acid for valine substitution at position 600. These mutations increase the basal kinase activity of B-Raf and are thought to uncouple Raf/MEK/ERK signalling from upstream proliferation drives including Ras and growth factor receptor activation resulting in constitutive activation of ERK. Mutated B-Raf proteins are transforming in NIH3T3 cells (Davies et al., Nature, 2002, 417, 949-954) and melanocytes (Wellbrock et al., Cancer Res., 2004, 64, 2338-2342) and have also been shown to be essential for melanoma cell viability and transformation (Hingorani et al., Cancer Res., 2003, 63, 5198-5202). As a key driver of the Raf/MEK/ERK signalling cascade, B-Raf represents a likely point of intervention in tumours dependent on this pathway.

AstraZeneca application WO 00/55153 discloses certain quinazolinones which are inhibitors of the production of cytokines such as tumour necrosis factor (TNF), in particular of TNFα, and various interleukins, in particular IL-1. The present inventors have surprisingly found that certain other, novel, quinazolinones are potent B-Raf inhibitors and are accordingly expected to be useful in the treatment of neoplastic disease.

Accordingly, the present invention provides a compound of formula (I):

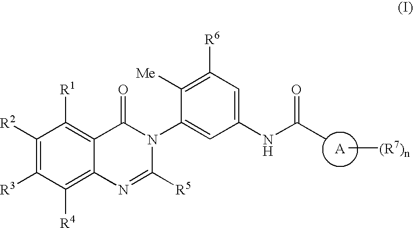

wherein:

Ring A is a 5 or 6 membered carbocyclyl or a 5 or 6 membered heterocyclyl; wherein if said heterocyclyl contains an —NH— moiety that nitrogen may be optionally substituted by a group selected from R²⁰;

R¹, R², R³, R⁴ and R⁵ are independently selected from hydrogen, halo, nitro, cyano, hydroxy, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂carbamoyl, N′—(C₁₋₆alkyl)ureido, N′,N′—(C₁₋₆alkyl)₂ureido, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁶or heterocyclyl-R¹⁶—; wherein at least one R¹, R², R³, R⁴ and R⁵ is not hydrogen; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹;

R⁶ is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂carbamoyl, C₁₋₆alkylS(O)a wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷- or heterocyclyl-R¹⁷—; wherein R⁶ may be optionally substituted on carbon by one or more R¹⁰; and wherein if said heterocyclyl contains an —NH— moiety that nitrogen may be optionally substituted by a group selected from R¹¹;

R⁷ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁸— or heterocyclyl-R¹⁸—; wherein R⁷ may be optionally substituted on carbon by one or more R¹²; and wherein if said heterocyclyl contains an —NH— moiety that nitrogen may be optionally substituted by a group selected from R¹³;

n is selected from 1-4; wherein the values of R⁷ may be the same or different;

R⁸, R¹⁰ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁹— or heterocyclyl-R¹⁹—; wherein R⁸, R¹⁰ and R¹² independently of each other may be optionally substituted on carbon by one or more R¹⁴; and wherein if said heterocyclyl contains an —NH— moiety that nitrogen may be optionally substituted by a group selected from R¹⁵;

R¹⁶, R¹⁷ and R¹⁹ are independently selected from a direct bond, —O—, —N(R²¹)—, —C(O)—, —N(R²¹)C(O)—, —C(O)N(R²¹)—, —S(O)ₛ—, —SO₂N(R²¹)— or —N(R²¹)SO₂—; wherein R²¹ is hydrogen or C₁₋₆alkyl and s is 0-2;

R¹⁸ is N(R²²)—, —C(O)—, —N(R²²)C(O)—, —C(O)N(R²²)—, —S(O)ₛ—, —SO₂N(R²²)— or —N(R²²)SO₂—; wherein R²² is hydrogen or C₁₋₆alkyl and s is 0-2;

R⁹, R¹¹, R¹³, R¹⁵ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that said compound is not: 2-chloro-N-{4-methyl-3-[6-(4-methylpiperazin-1-yl)-4-oxoquinazolin-3(4H)-yl]phenyl} isonicotinamide; 3,5-difluoro-N-{4-methyl-3-[6-(4-methylpiperazin-1-yl)-4-oxoquinazolin-3(4H)-yl]phenyl}benzamide; 3-(acetylamino)-N-{4-methyl-3-[6-(4-methylpiperazin-1-yl)-4-oxoquinazolin-3(4H)-yl]phenyl}benzamide; 3-fluoro-N-{4-methyl-3-[6-(4-methylpiperazin-1-yl)-4-oxoquinazolin-3(4H)-yl]phenyl}-4-(trifluoromethyl)benzamide; 2-methoxy-N-{4-methyl-3-[6-(4-methylpiperazin-1-yl)-4-oxoquinazolin-3(4H)-yl]phenyl}benzamide; 3-ethoxy-N-{4-methyl-3-[6-(4-methylpiperazin-1-yl)-4-oxoquinazolin-3(4H)-yl]phenyl}benzamide; N-{4-methyl-3-[6-(4-methylpiperazin-1-yl)-4-oxoquinazolin-3(4H)-yl]phenyl}-3-(1,1,2,2-tetrafluoroethoxy)benzamide; 3-chloro-N-{4-methyl-3-[6-(4-methyl-1,4-diazepan-1-yl)-4-oxoquinazolin-3(4H)-yl]phenyl}isonicotinamide; 3,5-difluoro-N-{4-methyl-3-[6-(4-methyl-1,4-diazepan-1-yl)-4-oxoquinazolin-3(4H)-yl]phenyl}benzamide; 4-methoxy-N-[4-methyl-3-(2-methyl-4-oxoquinazolin-3(4H)-yl)phenyl]benzamide; or 4-methyl-N-[4-methyl-3-(2-methyl-4-oxoquinazolin-3(4H)-yl)phenyl]benzamide.

According, to a further aspect of the present invention there is provided a compound of formula (I):

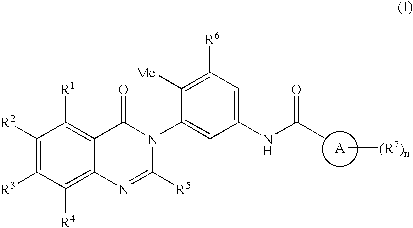

wherein:

Ring A is carbocyclyl or heterocyclyl; wherein if said heterocyclyl contains an —NH-moiety that nitrogen may be optionally substituted by a group selected from R²⁰;

R¹, R², R³, R⁴ and R⁵ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁶— or heterocyclyl-R¹⁶—; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹;

R⁶ is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)a wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁷—; wherein R⁶ may be optionally substituted on carbon by one or more R¹⁰; and wherein if said heterocyclyl contains an —NH— moiety that nitrogen may be optionally substituted by a group selected from R¹¹;

R⁷ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁸— or heterocyclyl-R¹⁸—; wherein R⁷ may be optionally substituted on carbon by one or more R¹²; and wherein if said heterocyclyl contains an —NH— moiety that nitrogen may be optionally substituted by a group selected from R¹³;

n is selected from 0-4; wherein the values of R⁷ may be the same or different;

R⁸, R¹⁰ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁹— or heterocyclyl-R¹⁹—; wherein R⁸, R¹⁰ and R¹² independently of each other may be optionally substituted on carbon by one or more R¹⁴; and wherein if said heterocyclyl contains an —NH— moiety that nitrogen may be optionally substituted by a group selected from R¹⁵;

R¹⁶, R¹⁷, R¹⁸ and R¹⁹ are independently selected from a direct bond, —O—, —N(R²¹)—, —C(O)—, —N(R²¹)C(O)—, —C(O)N(R²¹)—, —S(O)ₛ—, —SO₂N(R²¹)— or —N(R²¹)SO₂—; wherein R²¹ is hydrogen or C₁₋₆alkyl and s is 0-2;

R⁹, R¹¹, R¹³, R¹⁵ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, and a ring sulphur atom may be optionally oxidised to form the S-oxides. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. Further examples and suitable values of the term “heterocyclyl” are pyridyl, pyrrolyl, pyrimidinyl, pyrrolidinyl, pyrazolyl, piperidinyl, azetidinyl, 1,2,3-thiadiazolyl, 1,3,4-thiadiazolyl, morpholino, piperazinyl; oxiranyl, imidazolyl and tetrahydrofuranyl.

A “5 or 6 membered heterocyclyl” is a saturated, partially saturated or unsaturated, monocyclic ring containing 5 or 6 atoms of which at least one atom is chosen from nitrogen, sulphur or oxygen, which may, unless otherwise specified, be carbon or nitrogen linked, wherein a —CH₂— group can optionally be replaced by a —C(O)—, and a ring sulphur atom may be optionally oxidised to form the S-oxides. Examples and suitable values of the term “5 or 6 membered heterocyclyl” are morpholino, piperidyl, pyridyl, pyranyl, pyrrolyl, pyrazolyl, isothiazolyl, thienyl, thiadiazolyl, piperazinyl, thiazolidinyl, pyrrolidinyl, thiomorpholino, pyrrolinyl, 3,5-dioxapiperidinyl, tetrahydropyranyl, imidazolyl, pyrimidyl, pyrazinyl, pyridazinyl, isoxazolyl, 4-pyridone, 2-pyrrolidone and 4-thiazolidone.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl. A further particular example of “carbocyclyl” is cyclopropyl.

A “5 or 6 membered carbocyclyl” is a saturated, partially saturated or unsaturated, monocyclic carbon ring that contains 5 or 6 carbon atoms; wherein a —CH₂— group can optionally be replaced by a —C(O)—. Suitable values for “carbocyclyl” include cyclopropyl, cyclobutyl, 1-oxocyclopentyl, cyclopentyl, cyclopentenyl, cyclohexyl, cyclohexenyl and phenyl. A particular example of “5 or 6 membered carbocyclyl” is phenyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂carbamoyl” are N,N—(C₁₋₄alkyl)₂carbamoyl, dimethylaminocarbonyl and methylethylaminocarbonyl. Examples of “C₁₋₆alkylsulphonyl” are mesyl, ethylsulphonyl and isopropylsulphonyl. Examples of “C₁₋₆alkylsulphonylamino” are mesylamino, ethylsulphonylamino and isopropylsulphonylamino. Examples of “N′—(C₁₋₆alkyl)ureido” are N′-methylureido and N′-ethylureido. Examples of “N′,N′—(C₁₋₆alkyl)₂ureido” are N′,N′-dimethylureido and N′-methyl-N′-ethylureido.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess B-Raf inhibitory activity. The invention further relates to any and all tautomeric forms of the compounds of the formula (I) that possess B-Raf inhibitory activity.

It is also to be understood that certain compounds of the formula (I) can exist in solvated as well as unsolvated forms such as, for example, hydrated forms. It is to be understood that the invention encompasses all such solvated forms which possess B-Raf inhibitory activity.

Particular values of variable groups are as follows. Such values may be used where appropriate with any of the definitions, claims or embodiments defined hereinbefore or hereinafter.

Ring A is carbocyclyl.

Ring A is heterocyclyl; wherein if said heterocyclyl contains an —NH— moiety that nitrogen may be optionally substituted by a group selected from R²⁰.

Ring A is a 5 or 6 membered carbocyclyl.

Ring A is a 5 or 6 membered heterocyclyl; wherein if said heterocyclyl contains an —NH— moiety that nitrogen may be optionally substituted by a group selected from R²⁰.

Ring A is heterocyclyl; wherein if said heterocyclyl contains an —NH— moiety that nitrogen may be optionally substituted by a group selected from R²⁰; wherein R²⁰ is C₁₋₆alkyl.

Ring A is phenyl or pyrazolyl; wherein said pyrazolyl may be optionally substituted on nitrogen by a group selected from R²⁰; wherein R²⁰ is C₁₋₆alkyl.

Ring A is phenyl, pyridyl, thienyl or pyrazolyl; wherein said pyrazolyl may be optionally substituted on nitrogen by a group selected from R²⁰; wherein R²⁰ is C₁₋₆alkyl.

Ring A is phenyl or 1-t-butylpyrazolyl.

Ring A is phenyl, 1-t-butylpyrazol-5-yl, 1-methylpyrazol-5-yl, pyrid-2-yl, pyrid-3-yl, pyrid-4-yl, thien-2-yl and thien-3-yl.

Ring A is phenyl

Ring A is 1-t-butylpyrazolyl.

Ring A is 1-t-butylpyrazol-5-yl, 1-methylpyrazol-5-yl, pyrid-2-yl, pyrid-3-yl, pyrid-4-yl, thien-2-yl and thien-3-yl.

R¹, R², R³, R⁴ and R⁵ are independently selected from hydrogen, halo, nitro, cyano, hydroxy, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, N′—(C₁₋₆alkyl)ureido, N′,N′—(C₁₋₆alkyl)₂ureido, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino or carbocyclyl-R¹⁶—; wherein at least one R¹, R², R³, R⁴ and R⁵ is not hydrogen; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹.

R¹, R², R³, R⁴ and R⁵ are independently selected from hydrogen, halo, hydroxy, C₁₋₆alkyl, C₁₋₆alkoxy, N—(C₁₋₆alkyl)amino or heterocyclyl-R¹⁶—; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹; wherein

R⁸ is selected from hydroxy, N,N—(C₁₋₆alkyl)₂amino, N—(C₁₋₆alkyl)carbamoyl or heterocyclyl-R¹⁹—;

R¹⁶ and R¹⁹ are independently selected from a direct bond or —N(R²¹)—; wherein R²¹ is hydrogen; and

R⁹ is selected from C₁₋₆alkyl or C₁₋₆alkoxycarbonyl.

R¹, R², R³, R⁴ and R⁵ are independently selected from hydrogen, halo, hydroxy, amino, carboxy, carbamoyl, C₁₋₆alkyl, C₂₋₆alkynyl, C₁₋₆alkoxy, N—(C₁₋₆alkyl)amino, N—(C₁₋₆alkyl)carbamoyl, N′—(C₁₋₆alkyl)ureido, C₁₋₆alkylsulphonylamino, carbocyclyl-R¹⁶— or heterocyclyl-R¹⁶—; wherein at least one R¹, R², R³, R⁴ and R⁵ is not hydrogen; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹;

R⁸ is selected from hydroxy, amino, C₁₋₆alkyl, C₁₋₆alkoxy, N—(C₁₋₆alkyl)amino, N,N—(C₁₋₆alkyl)₂amino, C₁₋₆alkanoylamino, N—(C₁₋₆alkyl)carbamoyl or heterocyclyl-R¹⁹—; wherein R⁸, R¹⁰ and R¹² independently of each other may be optionally substituted on carbon by one or more R¹⁴; and wherein if said heterocyclyl contains an —NH— moiety that nitrogen may be optionally substituted by a group selected from R¹⁵;

R¹⁶ and R¹⁹ are independently selected from a direct bond, —N(R²¹)—, —N(R²¹)C(O)— or —C(O)N(R²¹)—; wherein R²¹ is hydrogen;

R⁹ and R¹⁵ are independently selected from C₁₋₆alkyl and C₁₋₆alkoxycarbonyl;

R¹⁴ is methoxy.

R¹, R², R³, R⁴ and R⁵ are independently selected from hydrogen, halo, hydroxy, C₁₋₆alkyl, C₁₋₆alkoxy, N—(C₁₋₆alkyl)amino, azetidinyl-R¹⁶—, pyrimidinyl-R¹⁶—, pyrazolyl-R¹⁶—, pyrrolyl-R¹⁶—, pyridyl-R¹⁶—, piperazinyl-R¹⁶— or morpholino-R¹⁶—; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹; wherein

R⁸ is selected from hydroxy, N,N—(C₁₋₆alkyl)₂amino, N—(C₁₋₆alkyl)carbamoyl, oxiranyl-R¹⁹—, piperidinyl-R¹⁹—, morpholino-R¹⁹—, pyridyl-R¹⁹— or pyrrolidinyl-R¹⁹—;

R¹⁶ and R¹⁹ are independently selected from a direct bond or —N(R²¹)—; wherein R²¹ is hydrogen; and

R⁹ is selected from C₁₋₆alkyl or C₁₋₆alkoxycarbonyl.

R¹, R², R³, R⁴ and R⁵ are independently selected from hydrogen, chloro, bromo, hydroxy, amino, carboxy, carbamoyl, methyl, propyl, propynyl, methoxy, ethoxy, propoxy, methylamino, ethylamino, propylamino, N-methylcarbamoyl, N-ethylcarbamoyl, N′-methylureido, mesylamino, cyclopropyl-R¹⁶—, pyridyl-R¹⁶—, pyrrolyl-R¹⁶—, pyrimidinyl-R¹⁶—, pyrrolidinyl-R¹⁶—, pyrazolyl-R¹⁶—, piperidinyl-R¹⁶—, azetidinyl-R¹⁶, 1,2,3-thiadiazolyl-R¹⁶—, 1,3,4-thiadiazolyl-R¹⁶—, morpholino-R¹⁶ or piperazinyl-R¹⁶—; wherein at least one R¹, R², R³, R⁴ and R⁵ is not hydrogen; wherein R¹, R², R³, R⁴ and R⁵ independently of each other may be optionally substituted on carbon by one or more R⁸; and wherein said piperazinyl may be optionally substituted by a group selected from R⁹;

R⁸ is selected from hydroxy, amino, methyl, methoxy, methylamino, dimethylamino, diethylamino, acetylamino, N-methylcarbamoyl, oxiranyl-R¹⁹—, morpholino-R¹⁹—, pyridyl-R¹⁹—, piperidinyl-R¹⁹—, piperazinyl-R¹⁹—, imidazolyl-R¹⁹—, tetrahydrofuranyl-R¹⁹— or pyrrolidinyl-R¹⁹—; wherein R⁸, R¹⁰ and R¹² independently of each other may be optionally substituted on carbon by one or more R¹⁴; and wherein said piperazinyl may be optionally substituted by a group selected from R¹⁵;

R¹⁶ and R¹⁹ are independently selected from a direct bond, —N(R²¹)—, —N(R²¹)C(O)— or —C(O)N(R²¹)—; wherein R²¹ is hydrogen;

R⁹ and R¹⁵ are independently selected from methyl, ethyl, isopropyl and t-butoxycarbonyl;

R¹⁴ is methoxy.

R¹, R², R³, R⁴ and R⁵ are independently selected from hydrogen, chloro, bromo, methyl, hydroxy, methoxy, pyrimidin-5-yl, pyrazol-4-yl, pyrrol-2-yl, pyrid-3-yl, morpholino, 4-ethylpiperazin-1-yl, azetidin-3-ylamino, 1-t-butoxycarbonylazetidin-3-ylamino, N-methylcarbamoylmethylamino, 2-pyrrolidin-1-ylethylamino, 2-pyrid-2-ylethylamino, 2-piperidin-1-ylethylamino, 2-hydroxypropylamino, 3-dimethylaminopropylamino, oxiran-2-ylmethoxy, 2-dimethylaminoethoxy, 2-pyrrolidin-1-ylethoxy, 2-morpholinoethoxy, 2-piperidin-1-ylethoxy, 3-dimethylaminopropoxy.

R¹, R², R³, R⁴ and R⁵ are independently selected from hydrogen, chloro, bromo, hydroxy, amino, carboxy, carbamoyl, methyl, 3-dimethylaminopropyl, 3-methylaminopropyl, 3-acetylaminopropyl, methoxy, N-methylcarbamoyl, N-(2-ethoxyethyl)carbamoyl, N-(2-dimethylaminoethyl)carbamoyl, N-[2-(imidazol-4-yl)ethyl]carbamoyl, 3-(amino)prop-1-yn-1-yl, 3-(acetylamino)prop-1-yn-1-yl, 3-(methylamino)prop-1-yn-1-yl, 3-(dimethylamino)prop-1-yn-1-yl, N′-methylureido, mesylamino, 2-(dimethylamino)ethoxy, 2-(diethylamino)ethoxy, 3-(dimethylamino)propoxy, 2-morpholinoethoxy, 3-morpholinopropoxy, 2-(piperidin-1-yl)ethoxy, 2-(pyrrolidino)ethoxy, oxiranylmethoxy, 3-(1-methylpiperazin-4-yl)propoxy, 2-(pyrrolidin-1-yl)ethylamino, 2-hydroxypropylamino, 2-(piperidin-1-yl)ethylamino, 3-(dimethylamino)propylamino, 2-(pyrid-2-yl)ethylamino, 1-(t-butoxycarbonyl)azetidin-3-ylamino, azetidin-3-ylamino, (N-methylcarbamoyl)methylamino, tetrahydrofuran-2-ylmethylamino, 2-methoxyethylamino, 3-(piperidin-1-yl)propylamino, cyclopropylaminocarbonyl, cyclopropylcarbonylamino, pyrazol-3-ylaminocarbonyl, 1,3,4-thiadiazol-2-ylaminocarbonyl, 5-methyl-1,3,4-thiadiazol-2-ylaminocarbonyl, 1,2,3-thiadiazol-4-ylcarbonylamino, 1-ethylpiperazin-4-yl, 1-isopropylpiperazin-4-yl, morpholino, azetidin-3-ylamino, pyrid-3-yl, pyrrol-2-yl, pyrazol-4-yl, pyrimidin-5-yl, 3-dimethylaminopyrrolidin-1-yl, 4-(piperidin-1-yl)piperidin-1-yl, (2S)-2-(methoxymethyl)pyrrolidin-1-yl and 1-methylpiperazin-4-yl.

R⁶ is hydrogen.

R⁷ is selected from C₁₋₆alkyl; wherein R⁷ may be optionally substituted on carbon by one or more R¹²; wherein R¹² is selected from halo or cyano.

R⁷ is a substituent on carbon and is selected from halo, C₁₋₆alkyl, C₁₋₆alkoxy, C₁₋₆alkylS(O)ₐ wherein a is 2, C₁₋₆alkylsulphonylamino, carbocyclyl-R¹⁸— or heterocyclyl-R¹⁸—; wherein R⁷ may be optionally substituted on carbon by one or more R¹²;

R¹² is selected from halo or cyano;

R¹⁸ is —S(O)ₛ— or —N(R²²)SO₂—; wherein R²² is hydrogen and s is 0-2.

R⁷ is selected from methyl, trifluoromethyl or 1-cyano-1-methylethyl.

R⁷ is selected from fluoro, chloro, methyl, t-butyl, methoxy, mesyl, cyclopropylaminosulphonyl, azetidin-1-ylsulphonyl, morpholinosulphonyl, mesylamino, trifluoromethyl or 1-cyano-1-methylethyl.

n is selected from 0-2; wherein the values of R⁷ may be the same or different.

n is selected from 0-1.

n is selected from 1 or 2; wherein the values of R⁷ may be the same or different.

n is 2; wherein the values of R⁷ may be the same or different.

n is 1.

n is 0.

Ring A, R⁷ and n together form 3-trifluoromethylphenyl, 3-(1-cyano-1-methylethyl)phenyl or 1-t-butyl-3-methylpyrazolyl.

Therefore in a further aspect of the invention there is provided a compound of formula (I) (as depicted above) wherein:

Ring A is carbocyclyl or heterocyclyl; wherein if said heterocyclyl contains an —NH-moiety that nitrogen may be optionally substituted by a group selected from R²⁰;

R¹, R², R³, R⁴ and R⁵ are independently selected from hydrogen, halo, hydroxy, C₁₋₆alkyl, C₁₋₆alkoxy, N—(C₁₋₆alkyl)amino or heterocyclyl-R¹⁶—; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹; wherein

R⁸ is selected from hydroxy, N,N—(C₁₋₆alkyl)₂amino, N—(C₁₋₆alkyl)carbamoyl or heterocyclyl-R¹⁹—;

R¹⁶ and R¹⁹ are independently selected from a direct bond or —N(R²¹)—; wherein R²¹ is hydrogen;

R⁹ is selected from C₁₋₆alkyl or C₁₋₆alkoxycarbonyl;

R⁶ is hydrogen;

R⁷ is selected from C₁₋₆alkyl; wherein R⁷ may be optionally substituted on carbon by one or more R¹²; wherein R¹² is selected from halo or cyano;

n is 1; and

R²⁰ is C₁₋₆alkyl;

or a pharmaceutically acceptable salt thereof.

Therefore in a further aspect of the invention there is provided a compound of formula (I) (as depicted above) wherein:

Ring A is carbocyclyl or heterocyclyl; wherein if said heterocyclyl contains an —NH-moiety that nitrogen may be optionally substituted by a group selected from R²⁰;

R¹, R², R³, R⁴ and R⁵ are independently selected from hydrogen, halo, hydroxy, C₁₋₆alkyl, C₁₋₆alkoxy, N—(C₁₋₆alkyl)amino or heterocyclyl-R¹⁶—; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹; wherein

R⁸ is selected from hydroxy, N,N—(C₁₋₆alkyl)₂amino, N—(C₁₋₆alkyl)carbamoyl or heterocyclyl-R¹⁹—;

R¹⁶ and R¹⁹ are independently selected from a direct bond or —N(R²¹)—; wherein R²¹ is hydrogen;

R⁹ is selected from C₁₋₆alkyl or C₁₋₆alkoxycarbonyl;

R⁶ is hydrogen;

R⁷ is selected from C₁₋₆alkyl; wherein R⁷ may be optionally substituted on carbon by one or more R¹²; wherein R¹² is selected from halo or cyano;

n is 1; and

R²⁰ is C₁₋₆alkyl;

or a pharmaceutically acceptable salt thereof;
with the proviso that said compound is not: 2-methyl-N-{4-methyl-3-[6-(4-methylpiperazin-1-yl)-4-oxoquinazolin-3(4H)-yl]phenyl}-2,3-dihydro-1-benzofuran-7-carboxamide; 2,2-dimethyl-N-{4-methyl-3-[6-(4-methylpiperazin-1-yl)-4-oxoquinazolin-3(4H)-yl]phenyl}chromane-6-carboxamide; or 4-methyl-N-[4-methyl-3-(2-methyl-4-oxoquinazolin-3(4H)-yl)phenyl]benzamide.

Therefore in a further aspect of the invention there is provided a compound of formula (I) (as depicted above) wherein:

Ring A is a 5 or 6 membered carbocyclyl or a 5 or 6 membered heterocyclyl; wherein if said heterocyclyl contains an —NH— moiety that nitrogen may be optionally substituted by a group selected from R²⁰;

R¹, R², R³, R⁴ and R⁵ are independently selected from hydrogen, halo, hydroxy, amino, carboxy, carbamoyl, C₁₋₆alkyl, C₂₋₆alkynyl, C₁₋₆alkoxy, N—(C₁₋₆alkyl)amino, N—(C₁₋₆alkyl)carbamoyl, N′—(C₁₋₆alkyl)ureido, C₁₋₆alkylsulphonylamino, carbocyclyl-R¹⁶ or heterocyclyl-R¹⁶—; wherein at least one R¹, R², R³, R⁴ and R⁵ is not hydrogen; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹;

R⁶ is hydrogen;

R⁷ is a substituent on carbon and is selected from halo, C₁₋₆alkyl, C₁₋₆alkoxy, C₁₋₆alkylS(O)ₐ wherein a is 2, C₁₋₆alkylsulphonylamino, carbocyclyl-R¹⁸— or heterocyclyl-R¹⁸—; wherein R⁷ may be optionally substituted on carbon by one or more R¹²;

n is selected from 1 or 2; wherein the values of R⁷ may be the same or different;

R⁸ is selected from hydroxy, amino, C₁₋₆alkyl, C₁₋₆alkoxy, N—(C₁₋₆alkyl)amino, N,N—(C₁₋₆alkyl)₂amino, C₁₋₆alkanoylamino, N—(C₁₋₆alkyl)carbamoyl or heterocyclyl-R¹⁹—; wherein R⁸, R¹⁰ and R¹² independently of each other may be optionally substituted on carbon by one or more R¹⁴; and wherein if said heterocyclyl contains an —NH— moiety that nitrogen may be optionally substituted by a group selected from R¹⁵;

R⁹ and R¹⁵ are independently selected from C₁₋₆alkyl and C₁₋₆alkoxycarbonyl;

R¹² is selected from halo or cyano;

R¹⁴ is methoxy;

R¹⁶ and R¹⁹ are independently selected from a direct bond, —N(R²¹)—, —N(R²¹)C(O)— or —C(O)N(R²¹)—; wherein R²¹ is hydrogen;

R¹⁸ is —S(O)ₛ— or —N(R²²)SO₂—; wherein R²² is hydrogen and s is 0-2;

R²⁰ is C₁₋₆alkyl;

or a pharmaceutically acceptable salt thereof;
with the proviso that said compound is not: 2-chloro-N-{4-methyl-3-[6-(4-methylpiperazin-1-yl)-4-oxoquinazolin-3(4H)-yl]phenyl} isonicotinamide; 3,5-difluoro-N-{4-methyl-3-[6-(4-methylpiperazin-1-yl)-4-oxoquinazolin-3(4H)-yl]phenyl}benzamide; 3-fluoro-N-{4-methyl-3-[6-(4-methylpiperazin-1-yl)-4-oxoquinazolin-3(4H)-yl]phenyl}-4-(trifluoromethyl)benzamide; 2-methoxy-N-{4-methyl-3-[6-(4-methylpiperazin-1-yl)-4-oxoquinazolin-3(4H)-yl]phenyl}benzamide; 3-ethoxy-N-{4-methyl-3-[6-(4-methylpiperazin-1-yl)-4-oxoquinazolin-3(4H)-yl]phenyl}benzamide; N-{4-methyl-3-[6-(4-methylpiperazin-1-yl)-4-oxoquinazolin-3(4H)-yl]phenyl}-3-(1,1,2,2-tetrafluoroethoxy)benzamide; 3-chloro-N-{4-methyl-3-[6-(4-methyl-1,4-diazepan-1-yl)-4-oxoquinazolin-3(4H)-yl]phenyl} isonicotinamide; 3,5-difluoro-N-{4-methyl-3-[6-(4-methyl-1,4-diazepan-1-yl)-4-oxoquinazolin-3(4H)-yl]phenyl}benzamide; 4-methoxy-N-[4-methyl-3-(2-methyl-4-oxoquinazolin-3(4H)-yl)phenyl]benzamide; or 4-methyl-N-[4-methyl-3-(2-methyl-4-oxoquinazolin-3(4H)-yl)phenyl]benzamide.

Therefore in a further aspect of the invention there is provided a compound of formula (I) (as depicted above) wherein:

Ring A is phenyl or 1-t-butylpyrazolyl;

R¹, R², R³, R⁴ and R⁵ are independently selected from hydrogen, chloro, bromo, methyl, hydroxy, methoxy, pyrimidin-5-yl, pyrazol-4-yl, pyrrol-2-yl, pyrid-3-yl, morpholino, 4-ethylpiperazin-1-yl, azetidin-3-ylamino, 1-t-butoxycarbonylazetidin-3-ylamino, N-methylcarbamoylmethylamino, 2-pyrrolidin-1-ylethylamino, 2-pyrid-2-ylethylamino, 2-piperidin-1-ylethylamino, 2-hydroxypropylamino, 3-dimethylaminopropylamino, oxiran-2-ylmethoxy, 2-dimethylaminoethoxy, 2-pyrrolidin-1-ylethoxy, 2-morpholinoethoxy, 2-piperidin-1-ylethoxy, 3-dimethylaminopropoxy;

R⁶ is hydrogen;

R⁷ is selected from methyl, trifluoromethyl or 1-cyano-1-methylethyl;

n is 1;

or a pharmaceutically acceptable salt thereof.

Therefore in a further aspect of the invention there is provided a compound of formula (I) (as depicted above) wherein:

Ring A is phenyl or 1-t-butylpyrazolyl;

R¹, R², R³, R⁴ and R⁵ are independently selected from hydrogen, chloro, bromo, methyl, hydroxy, methoxy, pyrimidin-5-yl, pyrazol-4-yl, pyrrol-2-yl, pyrid-3-yl, morpholino, 4-ethylpiperazin-1-yl, azetidin-3-ylamino, 1-t-butoxycarbonylazetidin-3-ylamino, N-methylcarbamoylmethylamino, 2-pyrrolidin-1-ylethylamino, 2-pyrid-2-ylethylamino, 2-piperidin-1-ylethylamino, 2-hydroxypropylamino, 3-dimethylaminopropylamino, oxiran-2-ylmethoxy, 2-dimethylaminoethoxy, 2-pyrrolidin-1-ylethoxy, 2-morpholinoethoxy, 2-piperidin-1-ylethoxy, 3-dimethylaminopropoxy;

R⁶ is hydrogen;

R⁷ is selected from methyl, trifluoromethyl or 1-cyano-1-methylethyl;

n is 1;

or a pharmaceutically acceptable salt thereof with the proviso that said compound is not: 4-methyl-N-[4-methyl-3-(2-methyl-4-oxoquinazolin-3(4H)-yl)phenyl]benzamide.

Therefore in a further aspect of the invention there is provided a compound of formula (I) (as depicted above) wherein:

Ring A is phenyl, 1-t-butylpyrazol-5-yl, 1-methylpyrazol-5-yl, pyrid-2-yl, pyrid-3-yl, pyrid-4-yl, thien-2-yl and thien-3-yl;

R¹, R², R³, R⁴ and R⁵ are independently selected from hydrogen, chloro, bromo, hydroxy, amino, carboxy, carbamoyl, methyl, 3-dimethylaminopropyl, 3-methylaminopropyl, 3-acetylaminopropyl, methoxy, N-methylcarbamoyl, N-(2-ethoxyethyl)carbamoyl, N-(2-dimethylaminoethyl)carbamoyl, N-[2-(imidazol-4-yl)ethyl]carbamoyl, 3-(amino)prop-1-yn-1-yl, 3-(acetylamino)prop-1-yn-1-yl, 3-(methylamino)prop-1-yn-1-yl, 3-(dimethylamino)prop-1-yn-1-yl, N′-methylureido, mesylamino, 2-(dimethylamino)ethoxy, 2-(diethylamino)ethoxy, 3-(dimethylamino)propoxy, 2-morpholinoethoxy, 3-morpholinopropoxy, 2-(piperidin-1-yl)ethoxy, 2-(pyrrolidino)ethoxy, oxiranylmethoxy, 3-(1-methylpiperazin-4-yl)propoxy, 2-(pyrrolidin-1-yl)ethylamino, 2-hydroxypropylamino, 2-(piperidin-1-yl)ethylamino, 3-(dimethylamino)propylamino, 2-(pyrid-2-yl)ethylamino, 1-(t-butoxycarbonyl)azetidin-3-ylamino, azetidin-3-ylamino, (N-methylcarbamoyl)methylamino, tetrahydrofuran-2-ylmethylamino, 2-methoxyethylamino, 3-(piperidin-1-yl)propylamino, cyclopropylaminocarbonyl, cyclopropylcarbonylamino, pyrazol-3-ylaminocarbonyl, 1,3,4-thiadiazol-2-ylaminocarbonyl, 5-methyl-1,3,4-thiadiazol-2-ylaminocarbonyl, 1,2,3-thiadiazol-4-ylcarbonylamino, 1-ethylpiperazin-4-yl, 1-isopropylpiperazin-4-yl, morpholino, azetidin-3-ylamino, pyrid-3-yl, pyrrol-2-yl, pyrazol-4-yl, pyrimidin-5-yl, 3-dimethylaminopyrrolidin-1-yl, 4-(piperidin-1-yl)piperidin-1-yl, (2S)-2-(methoxymethyl)pyrrolidin-1-yl and 1-methylpiperazin-4-yl;

R⁶ is hydrogen;

R⁷ is selected from fluoro, chloro, methyl, t-butyl, methoxy, mesyl, cyclopropylaminosulphonyl, azetidin-1-ylsulphonyl, morpholinosulphonyl, mesylamino, trifluoromethyl or 1-cyano-1-methylethyl;

n is selected from 1 or 2; wherein the values of R⁷ may be the same or different;

or a pharmaceutically acceptable salt thereof;
with the proviso that said compound is not: 2-chloro-N-{4-methyl-3-[6-(4-methylpiperazin-1-yl)-4-oxoquinazolin-3(4H)-yl]phenyl}isonicotinamide; 3,5-difluoro-N-{4-methyl-3-[6-(4-methylpiperazin-1-yl)-4-oxoquinazolin-3(4H)-yl]phenyl}benzamide; 2-methoxy-N-{4-methyl-3-[6-(4-methylpiperazin-1-yl)-4-oxoquinazolin-3(4H)-yl]phenyl}benzamide; 4-methoxy-N-[4-methyl-3-(2-methyl-4-oxoquinazolin-3(4H)-yl)phenyl]benzamide; or 4-methyl-N-[4-methyl-3-(2-methyl-4-oxoquinazolin-3(4H)-yl)phenyl]benzamide.

In another aspect of the invention, preferred compounds of the invention are any one of the Examples or a pharmaceutically acceptable salt thereof.

In another aspect of the invention, particular compounds of the invention are any one of Examples 49, 58, 59, 62, 66, 71, 74, 81, 86, 97, 107 and 108 or a pharmaceutically acceptable salt thereof.

Another aspect of the present invention provides a process for preparing a compound of formula (I) or a pharmaceutically acceptable salt thereof which process (wherein variable are, unless otherwise specified, as defined in formula (I)) comprises of:

### Process a) Reacting an Amine of the Formula (II)

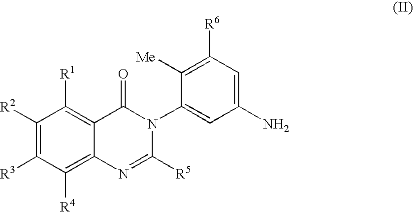

with an acid of formula (III):

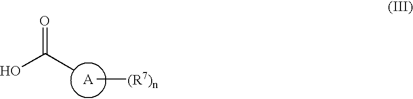

or an activated acid derivative thereof;

### Process b) Reacting an Amine of Formula (VI):

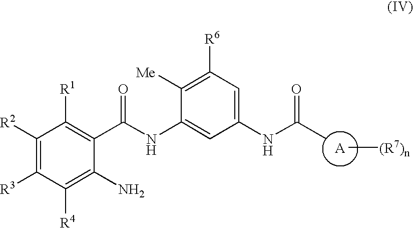

with an compound of formula (V):

(R^{a}O)₃CR⁵ (V)

wherein R^{a} is methyl or ethyl;

### Process c) Reaction of an Amine of Formula (VI):

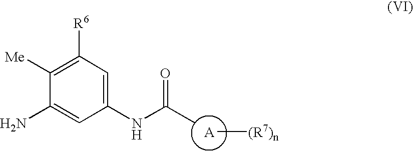

with a benzo[d][1,3]oxazin-4-one of the formula (VII):

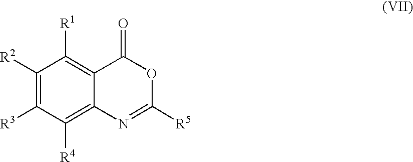

and thereafter if necessary:
i) converting a compound of the formula (I) into another compound of the formula (I);
ii) removing any protecting groups;
iii) forming a pharmaceutically acceptable salt.

Specific reaction conditions for the above reactions are as follows.

Process a) Amines of formula (II) and acids of formula (III) may be coupled together in the presence of a suitable coupling reagent. Standard peptide coupling reagents known in the art can be employed as suitable coupling reagents, or for Example carbonyldiimidazole and dicyclohexyl-carbodiimide, optionally in the presence of a catalyst such as dimethylaminopyridine or 4-pyrrolidinopyridine, optionally in the presence of a base for Example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for Example acid chlorides, and active esters, for Example pentafluorophenyl esters. The reaction of these types of compounds with amines is well known in the art, for Example they may be reacted in the presence of a base, such as those described above, and in a suitable solvent, such as those described above. The reaction may conveniently be performed at a temperature in the range of −40 to 40° C.

Amines of formula (II) may be prepared according to Scheme 1:

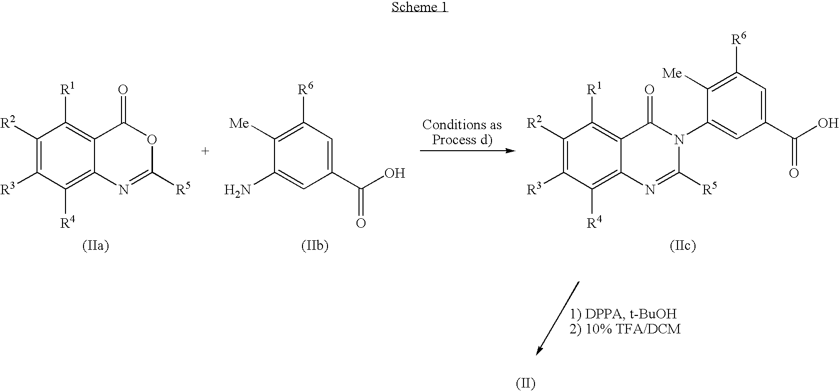

Compounds of formula (IIa), (IIb) and (III) are commercially available compounds, or they are known in the literature or they may be prepared by standard processes known in the art.

Process b) Compounds of formula (IV) and (V) can be reacted in an appropriate solvent with a catalyst such as acetic acid. For example, compounds of formula (IV) and (V) can be heated in the presence of ethanol and catalytic acetic acid to yield compounds of formula (I). Suitable solvents include toluene, benzene, and isopropyl alcohol.

Amines of formula (IV) may be prepared according to Scheme 2:

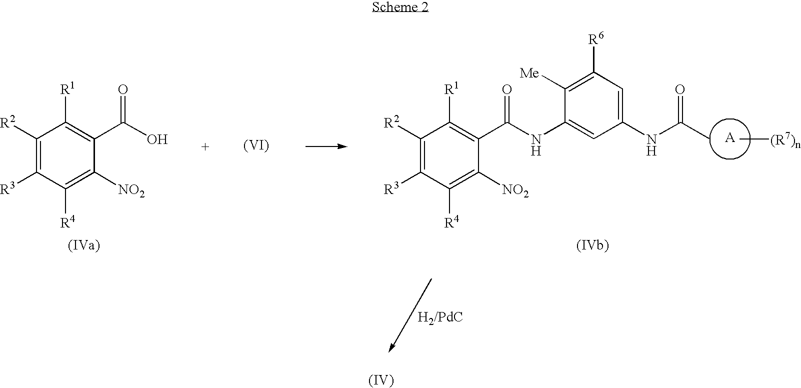

Compounds of formula (IVa) and (V) are commercially available compounds, or they are known in the literature or they may be prepared by standard processes known in the art. By utilizing

Process c) Compounds of formula (VII) and (VI) can be heated together in an appropriate solvent. For example, compounds of formula (VII) and (VI) can be heated in the presence of DMF. Other suitable solvents include toluene, benzene and dioxane.

Amines of formula (VI) may be prepared according to Scheme 3:

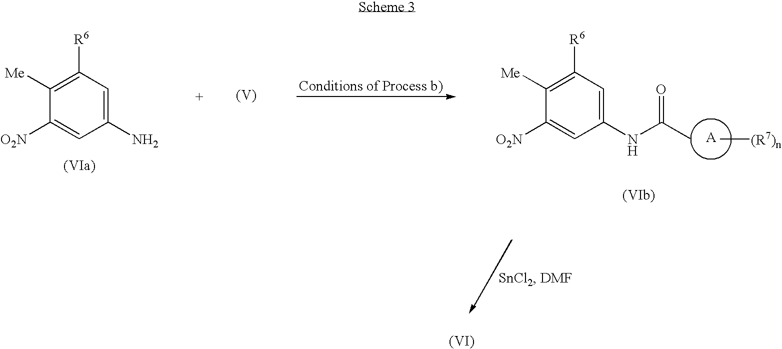

Compounds of formula (VII) and (VIa) are commercially available compounds, or they are known in the literature or they may be prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cancer activity which is believed to arise from the B-Raf inhibitory activity of the compound. These properties may be assessed, for example, using the procedure set out below:—

### B-Raf In Vitro ELISA Assay

Activity of human recombinant, purified wild type His-B-Raf protein kinase was determined in vitro using an enzyme-linked immunosorbent assay (ELISA) assay format, which measures phosphorylation of the B-Raf substrate, human recombinant, purified His-derived (detagged) MEK1. The reaction utilized 2.5 nM B-Raf, 0.15 μM MEK1 and 10 μM adenosine triphosphate (ATP) in 40 mM N-(2-hydroxyethyl)piperazine-N′-(2-ethanesulfonic acid hemisodium salt (HEPES), 5 mM 1,4-dithio-DL-threitol (DTT), 10 mM MgCl₂, 1 mM ethylenediaminetetraacetic acid (EDTA) and 0.2 M NaCl (1×HEPES buffer), with or without compound at various concentrations, in a total reaction volume of 25 μl in 384 well plates. B-Raf and compound were preincubated in 1×HEPES buffer for 1 hour at 25° C. Reactions were initiated with addition of MEK1 and ATP in 1×HEPES buffer and incubated at 25° C. for 50 minutes and reactions stopped by addition of 10 μl 175 mM EDTA (final concentration 50 mM) in 1×HEPES buffer. 5 μl of the assay mix was then diluted 1:20 into 50 mM EDTA in 1×HEPES buffer, transferred to 384 well black high protein binding plates and incubated overnight at 4° C. Plates were washed in tris buffered saline containing 0.1% Tween20 (TBST), blocked with 50 μl Superblock (Pierce) for 1 hour at 25° C., washed in TBST, incubated with 50 μl rabbit polyclonal anti-phospho-MEK antibody (Cell Signaling) diluted 1:1000 in TBS for 2 hours at 25° C., washed with TBST, incubated with 50 μl goat anti-rabbit horseradish peroxidase-linked antibody (Cell Signaling) diluted 1:2000 in TBS for 1 hour at 25° C. and washed with TBST. 50 μl of fluorogenic peroxidase substrate (Quantablu—Pierce) was added and following incubation for 45-60 minutes, 50 ul QuantabluSTOP (Pierce) was added. Blue fluorescent product was detected at excitation 325 and emission 420 using a TECAN Ultra plate reader. Data was graphed and IC₅₀s calculated using Excel Fit (Microsoft).

When tested in the above in vitro assay, the compounds of the present invention exhibited activity less than 30 μM. For example the following results were obtained:

| Example No | IC₅₀ (μM) |
|---|---|
| 9 | 0.535 |
| 14 | 3.20 |
| 22 | 0.518 |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their B-Raf inhibitory properties. Accordingly the compounds of the present invention are expected to be useful in the treatment of diseases or medical conditions mediated alone or in part by B-Raf, i.e. the compounds may be used to produce a B-Raf inhibitory effect in a warm-blooded animal in need of such treatment.

Thus the compounds of the present invention provide a method for treating cancer characterised by inhibition of B-Raf, i.e. the compounds may be used to produce an anti-cancer effect mediated alone or in part by the inhibition of B-Raf.

Such a compound of the invention is expected to possess a wide range of anti-cancer properties as activating mutations in B-Raf have been observed in many human cancers, including but not limited to, melanoma, papillary thyroid tumors, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those primary and recurrent solid tumours which are associated with B-Raf, especially those tumours which are significantly dependent on B-Raf for their growth and spread, including for example, certain tumours of the skin, colon, thyroid, lungs and ovaries. Particularly the compounds of the present invention are useful in the treatment of melanomas.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a B-Raf inhibitory effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

The B-Raf inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
(x) Cell cycle inhibitors including for example CDK inhibitiors (eg flavopiridol) and other inhibitors of cell cycle checkpoints (e.g. checkpoint kinase); inhibitors of aurora kinase and other kinases involved in mitosis and cytokinesis regulation (e.g. mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of B-Raf in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are the millimolar ratio equivalents to those used in the previous example;
(xi) the following abbreviations have been used:

THF tetrahydrofuran;

DMF N,N-dimethylformamide;

EtOAc ethyl acetate;

Pd₂(dba)₃ tris(dibenzylideneacetone)dipalladium (0);

BINAP (+/−)-2,2′-bis(diphenylphosphino)-1,1′-binaphthyl;

EDCI 1-(3-dimethylaminopropyl)-3-ethylcarbodiimide hydrochloride;

HOBt hydroxybenzotriazole;

DCM dichloromethane; and

DMSO dimethylsulphoxide;

(xii) “ISCO” refers to normal phase flash column chromatography using 12g and 40 g pre-packed silica gel cartridges used according to the manufacturers instruction obtained from ISCO, Inc, 4700 superior street Lincoln, Nebr., USA.; and
(xiii) “Reverse phase Gilson” refers to a YMC-AQC18 reverse phase HPLC Column with dimension 20mm/100 and 50 mm/250 in water/acetonitrile with 0.1% TFA as mobile phase,obtained from Waters Corporation 34, Maple street, Milford Mass., USA.

### Example 1

### N-[3-(6-Bromo-4-oxo-4H-quinazolin-3-yl)-4-methylphenyl]-3-trifluoromethylbenzamide

A stirred mixture of 2-amino-5-bromobenzoic acid (646 mg, 2.99 mmol), triethyl orthoformate (738 μl, 4.49 mmol) and acetic acid (17 μl, 0.30 mmol) in toluene (13 ml) was heated under reflux for 2.5 hours. N-(3-Amino-4-methylphenyl)-3-trifluoromethylbenzamide (Method 2; 879 mg, 2.99 mmol) was then added to the mixture and stirred at 120° C. for 16 hours. The mixture was cooled to 25° C. and the resulting precipitate was collected by vacuum filtration and dried to give 750 mg (50%) of a white solid. NMR (400 MHz): 10.70 (s, 1H), 7.50-8.45 (m, 11H), 2.12 (s, 3H); m/z 503.

### Examples 2-4

The following compounds were prepared by the procedure of Example 1, using N-(3-amino-4-methylphenyl)-3-trifluoromethylbenzamide (Method 2) or N-(3-amino-4-methylphenyl)-3-(cyano-dimethyl-methyl)-benzamide (Method 15) and the appropriate starting material.

| Ex | Compound | NMR | m/z | S.M |
|---|---|---|---|---|
| 2 | 3-[2-Methyl-5-(3- | 14.75 (s, br, 1H), 10.75 (s, | 467 | 2-Amino- |
| | trifluoromethyl- | 1H), 8.85 (s, 1H), 8.55 (m, | | isophthalic acid |
| | benzoylamino)-phenyl]-4- | 2H), 8.30 (m, 2H), 8.01 (m, | | |
| | oxo-3,4-dihydro- | 2H), 7.70 (m, 3H), 7.55 (d, | | |
| | quinazoline-8-carboxylic | 1H), 2.20 (s, 3H) | | |
| | acid | | | |
| 3 | 3-(5-{[3-(1-Cyano-1- | 14.75 (s, 1H), 10.66 (s, | 467 | 2-Amino- |
| | methylethyl)benzoyl]amino}- | 1H), 8.82 (s, 1H), 8.55 (d, | | isophthalic acid |
| | 2-methylphenyl)-4-oxo-3,4- | 2H), 8.15 (s, 1H), 8.00 (m, | | |
| | dihydroquinazoline-8- | 2H), 7.90 (m, 3H), 7.70 (s, | | |
| | carboxylic acid | 1H), 7.55 (d, 1H), 2.10 (s, | | |
| | | 3H), 1.85 (s, 6H) | | |
| 4 | 6-Bromo-3-{5-[3-(cyano- | 14.50 (s, br, 1H), 10.60 (s, | 546 | 2-Amino-5- |
| | dimethyl-methyl)- | 1H), 8.70 (s, 1H), 8.51 (s, | | bromo-isophthalic |
| | benzoylamino]-2-methyl- | 1H), 8.50 (s, 1H), 8.10 (s, | | acid |
| | phenyl}-4-oxo-3,4-dihydro- | 1H), 8.00 (m, 2H), 7.90 (m, | | |
| | quinazoline-8-carboxylic | 2H), 7.70 (t, 1H), 7.50 (d, | | |
| | acid | 1H), 2.10 (s, 3H), 1.70 (s, | | |
| | | 6H) | | |

### Example 5

### N-[4-Methyl-3-(6-morpholino-4-oxo-4H-quinazolin-3-yl)phenyl]-3-trifluoromethylbenzamide

A microwave vial was charged with sodium tert-butoxide (33 mg, 0.299 mmol), Pd₂(dba)₃ (18 mg, 10% mmol), BINAP (24 mg, 20% mmol) and N-[3-(6-bromo-4-oxo-4H-quinazolin-3-yl)-4-methylphenyl]-3-trifluoromethylbenzamide (Example 1; 100 mg, 0.199 mmol). The vial was fitted with a septum and purged with nitrogen. 1,4-Dioxane (3.3 ml) and morpholine (21 mg, 0.239 mmol, 1.2 eq) were then added via syringe. The vial was irradiated in a microwave at 175° C. for 30 min. The reaction mixture was filtered through a pad of silica gel and washed with DCM. The filtrate was concentrated and the residue was purified by purified by column chromatography utilizing an ISCO system (hexane-EtOAc) to give 35 mg (34.7%) of light yellow solid. NMR (400 MHz): 10.55 (s, 1H), 7.40-8.25 (m, 11), 3.72 (m, 4H), 3.17 (m, 4H), 2.00 (s, 3H); m/z 509.

### Examples 6-10

The following compounds were prepared by the procedure of Example 5, using the appropriate amine and N-[3-(6-bromo-4-oxo-4H-quinazolin-3-yl)-4-methylphenyl]-3-trifluoromethylbenzamide (Example 1) as a starting material.

| Ex | Compound | NMR | m/z |
|---|---|---|---|
| 6 | N-{4-Methyl-3-[4-oxo-6-(2- | 10.55 (s, 1H), 8.20 (m, 2H), 7.96 (s, 1H), | 536 |
| | pyrrolidin-1-yl-ethylamino)- | 7.90 (d, 1H), 7.72 (m, 3H), 7.50 (d, 1H), | |
| | 4H-quinazolin-3-yl]phenyl}-3- | 7.32 (d, 1H), 7.20 (m, 2H), 3.42 (m, 4H), | |
| | trifluoromethylbenzamide | 3.25 (m, 2H), 3.00 (m, 2H), 1.95 (m, 5H), | |
| | | 1.85 (m, 2H) | |
| 7 | N-{3-[6-(2-Hydroxy- | 10.70 (s, 1H), 8.35 (s, 1H), 8.30 (d, 1H), | 497 |
| | propylamino)-4-oxo-4H- | 8.05 (m, 2H), 7.85 (m, 3H), 7.55 (d, 1H), | |
| | quinazolin-3-yl]-4- | 7.50 (d, 1H), 7.30 (d, 1H), 7.20 (s, 1H), | |
| | methylphenyl}-3- | 6.32 (t, 1H), 4.80 (d, 1H), 3.89 (m, 1H), 3.10 (m, | |
| | trifluoromethylbenzamide | 2H), 2.10 (s, 3H), 1.20 (d, 3H) | |
| 8 | N-{4-Methyl-3-[4-oxo-6-(2- | 10.50 (s, 1H), 8.12 (s, 1H), 8.10 (d, 1H), | 550 |
| | piperidin-1-yl-ethylamino)- | 7.90 (s, 1H), 7.82 (d, 1H), 7.65 (m, 3H), | |
| | 4H-quinazolin-3-yl]phenyl}-3- | 7.40 (d, 1H), 7.30 (d, 1H), 7.10 (s, 2H), | |
| | trifluoromethylbenzamide | 3.30 (m, 4H), 3.10 (m, 2H), 2.80 (m, 2H), | |
| | | 1.90 (s, 3H), 1.50-1.70 (m, 5H), 1.22 (m, 1H) | |
| 9 | N-{3-[6-(3-Dimethylamino | 10.60 (s, 1H), 8.23 (s, 1H), 8.22 (d, 1H), | 524 |
| | propylamino)-4-oxo-4H- | 7.95 (m, 2H), 7.75 (m, 3H), 7.70 (br, 1H), | |
| | quinazolin-3-yl]-4- | 7.50 (d, 1H), 7.40 (d, 1H), 7.30 (d, 1H), | |
| | methylphenyl}-3- | 7.22 (s, 1H), 3.10 (m, 4H), 2.80 (m, 2H), | |
| | trifluoromethylbenzamide | 2.05 (s, 3H), 1.00 (s, 6H) | |
| 10 | N-{4-Methyl-3-[4-oxo-6-(2- | 10.35 (s, 1H), 8.55 (s, 1H), 8.10 (m, 3H), | 546 |
| | pyridin-2-yl-ethylamino)-4H- | 7.76 (m, 2H), 7.60 (m, 6H), 7.35 (d, 1H), | |
| | quinazolin-3-yl]phenyl}-3- | 7.22 (d, 1H), 7.00 (m, 2H), 3.35 (t, 2H), | |
| | trifluoromethylbenzamide | 3.03 (t, 2H), 1.85 (s, 3H) | |

### Example 11

### N-[3-(6-Bromo-2-methyl-4-oxo-4H-quinazolin-3-yl)-4-methylphenyl]-3-trifluoromethylbenzamide

A suspension of 6-bromo-2-methylbenzo[d][1,3]oxazin-4-one (240 mg, 1 mmol) and N-(3-amino-4-methylphenyl)-3-trifluoromethylbenzamide (Method 2; 294 mg, 1 mmol) in 5 ml of anhydrous toluene was heated at reflux for 12 hours. The resulting solid was collected by vacuum filtration, washed with EtOAc:hexane (1:1) and dried (280 mg, 54.2%). NMR (400 MHz): 10.62 (s, 1H), 8.35 (s, 1H), 8.30 (d, 1H), 8.10 (d, 1H), 8.05 (d, 1H), 8.00 (s, 1H), 7.85 (m, 3H), 7.79 (d, 1H), 7.52 (d, 1H), 2.20 (s, 3H), 2.10 (s, 3H); m/z 517.

### Example 12

The following compound was prepared by the procedure of Example 11, using 7-bromo-2-methylbenzo[d][1,3]oxazin-4-one

| Ex | Compound | m/z |
|---|---|---|
| 12 | N-[3-(7-bromo-2-methyl-4-oxoquinazolin-3(4H)-yl)-4- | 517 |
| | methylphenyl]-3-(trifluoromethyl)benzamide | |

### Example 13

### N-{3-[6-(4-Ethylpiperazin-1-yl)-2-methyl-4-oxo-4H-quinazolin-3-yl]-4-methylphenyl}-3-trifluoromethylbenzamide

A microwave vial was charged with sodium tert-butoxide (32 mg, 0.291 m mol), Pd₂ (dba)₃ (18 mg, 10% m mol), BINAP (24 mg, 20% m mol) and N-[3-(6-bromo-2-methyl-4-oxo-4H-quinazolin-3-yl)-4-methylphenyl]-3-trifluoromethylbenzamide (Example 11; 100 mg, 0.194 m mol). The vial was fitted with a septum and purged with nitrogen. 1-Ethyl-piperazine (53 mg, 0.465 mmol, 2.4 eq) in 1,4-dioxane (3.3 ml) was then added via syringe. The vial irradiated in a microwave at 175° C. for 30 min. The mixture was filtered through a pad of silica gel and washed with DCM. The filtrate was concentrated and then purified by column chromatography utilizing an ISCO system (hexane-EtOAc to 0.1% triethyl amine and 5% methanol in DCM) to give 35 mg (32.8%) of a light yellow solid. NMR (400 MHz): 10.40 (s, 1H), 8.10 (m, 2H), 7.80 (d, 1H), 7.55 (m, 3H), 7.40 (m, 2H), 7.20 (m, 2H), 3.20 (m, 4H), 3.10 (m, 4H), 2.20 (q, 2H), 1.90 (s, 3H), 1.80 (s, 3H), 0.85 (t, 3H); m/z 550.

### Examples 14-15

The following examples were synthesised by the procedure of Example 13 using N-[3-(6-bromo-2-methyl-4-oxo-4H-quinazolin-3-yl)-4-methylphenyl]-3-trifluoromethylbenzamide (Example 11) or N-[3-(7-bromo-2-methyl-4-oxoquinazolin-3(4H)-yl)-4-methylphenyl]-3-(trifluoromethyl)benzamide (Example 12) and the appropriate amine as starting materials.

| Ex | Compound | NMR | M/z |
|---|---|---|---|
| 14 | N-[4-Methyl-3-(2-methyl-6- | 10.70 (s, 1H) 8.32 (m, 2H), 8.05 (s, 1H), | 523 |
| | morpholin-4-yl-4-oxo-4H- | 7.80 (m, 3H), 7.65 (m, 2H), 7.50 (m, | |
| | quinazolin-3-yl)phenyl]-3- | 2H), 3.80 (m, 4H), 3.20 (m, 4H), | |
| | trifluoromethylbenzamide | 2.20 (s, 3H), 2.13 (s, 3H) | |
| 15 | N-{3-[7-(4-Ethyl-piperazin- | 10.40 (s, 1H) 8.10 (m, 1H), 8.05 (d, | 550 |
| | 1-yl)-2-methyl-4-oxo-4H- | 1H), 7.80 (m, 2H), 7.65 (m, 3H), | |
| | quinazolin-3-yl]-4- | 7.25 (d, 1H), 7.10 (d, 1H), 6.90 (s, 1H), | |
| | methylphenyl}-3- | 4.00 (m, 2H), 3.40 (m, 2H), 3.00 (m, 6H), 1.95 | |
| | trifluoromethylbenzamide | (s, 3H), 1.80 (s, 3H), 1.10 (t, 3H) | |

### Example 16

### N-[3-(7-Bromo-4-oxo-4H-quinazolin-3-yl)-4-methylphenyl]-3-trifluoromethylbenzamide

A stirred mixture of 2-amino-4-bromobenzoic acid (Method 3; 607 mg, 2.8 mmol), triethyl orthoformate (622 mg, 700 μl, 4.2 mmol) and acetic acid (17 μl, 0.30 mmol) in toluene (13 ml) was heated at reflux for 2.5 hours. N-(3-Amino-4-methylphenyl)-3-trifluoromethylbenzamide (Method 2; 827 mg, 2.8 mmol) was then added and the mixture was stirred at 120° C. for 16 hours. The solvent was removed under reduced pressure to 5-8 ml and cooled to 25° C. The resulting precipitate was filtered, washed with EtOAc:hexane (1:1), and dried in vacuo to yield 671 mg (47.8%) of white solid. NMR (400 MHz): 10.70 (s, 1H), 8.42 (s, 1H), 8.35 (m, 2H), 8.20 (d, 1H), 8.05 (m, 2H), 7.95 (s, 1H), 7.85 (m, 3H), 7.50 (d, 1H), 2.11 (s, 3H); m/z 503.

### Example 17

### N-[4-Methyl-3-(7-morpholin-4-yl-4-oxo-4H-quinazolin-3-yl)phenyl]-3-trifluoromethylbenzamide

A microwave vial was charged with sodium tert-butoxide (33 mg, 0.299 mmol), Pd₂(dba)₃ (18 mg, 10% mmol), BINAP (24 mg, 20% mmol) and N-[3-(7-bromo-4-oxo-4H-quinazolin-3-yl)-4-methylphenyl]-3-trifluoromethylbenzamide (Example 16; 100 mg, 0.199 mmol). The vial was fitted with a septum and purged with nitrogen. Morpholine (42 mg, 0.478 mmol, 2.4 eq) in 1,4-dioxane was then added via syringe. The vial irradiated in a microwave at 175° C. for 30 min. The mixture was filtered through a pad of silica gel and washed with DCM. The filtrate was concentrated and then purified by column chromatography utilizing an ISCO system (hexane-EtOAc) to give 40 mg (39.6%) of a light yellow solid. NMR (400 MHz): 10.56 (s, 1H), 8.23 (s, 1H), 8.22 (d, 1H), 8.15 (s, 1H), 7.95 (m, 2H), 7.79 (m, 3H), 7.31 (d, 1H), 7.22 (d, 1H), 7.00 (s, 1H), 3.70 (m, 4H), 3.30 (m, 4H), 2.05 (s, 3H); m/z 509.

### Example 18

The following compound was prepared according to Example 17 using N-[3-(7-bromo-4-oxo-4H-quinazolin-3-yl)-4-methylphenyl]-3-trifluoromethylbenzamide (Example 16) and the appropriate amine as starting materials.

| Ex. | Compound | NMR | m/z |
|---|---|---|---|
| 18 | N-{3-[7-(4-Ethyl-piperazin- | 10.50 (s, 1H), 8.16 (m, 3H), 7.98 (d, 1H), | 536 |
| | 1-yl)-4-oxo-4H-quinazolin- | 7.90 (d, 1H), 7.70 (m, 3H), 7.35 (d, 1H), 7.25 (d, | |
| | 3-yl]-4-methylphenyl}-3- | 1H), 7.06 (s, 1H), 4.10 (d, 2H), 3.50 (d, 2H), | |
| | trifluoromethylbenzamide | 3.10 (m, 6H), 1.65 (s, 3H), 1.19 (t, 3H) | |

### Example 19

### N-[3-(6-Methoxy-4-oxo-4H-quinazolin-3-yl)-4-methylphenyl]-3-trifluoromethylbenzamide

A stirred mixture of 5-methoxyanthranilic acid (500 mg, 2.99 mmol), trimethylorthoformate (491 μl, 4.49 mmol) and acetic acid (17 μl, 0.30 mmol) in toluene (13 ml) was heated under at for 2.5 hours. N-(3-Amino-4-methylphenyl)-3-trifluoromethylbenzamide (Method 2; 750 mg, 3 mmol) was then added to the reaction mixture and heating was continued for 16 hours. The reaction mixture was cooled to 25° C. and diluted with EtOAc. The solution was then washed with 1 M HCl, 2 M NaOH, brine, and dried with Na₂SO₄(s). The solvents were removed under reduced pressure to yield a cream coloured foam/solid (731 mg, 70% crude yield based on aniline). The product was purified by column chromatography utilizing an ISCO system (EtOAc/Hexane) to give 558 mg (53%) an off white solid. NMR (400 MHz): 10.57 (s, 1H), 7.50-8.45 (m, 11H), 3.59 (s, 3H), 2.12 (s, 3H); m/z 454.

### Example 20

### N-{3-[6-(2-Dimethylamino-ethoxy)-4-oxo-4H-quinazolin-3-yl]-4-methylphenyl}-3-trifluoromethylbenzamide

A suspension of N-[3-(6-hydroxy-4-oxo-4H-quinazolin-3-yl)-4-methylphenyl]-3-trifluoromethylbenzamide (Example 52; 100 mg, 0.228 mmol), 2-dimethyl amino ethyl chloride hydrochloride (43 mg, 0.296 mmol), potassium carbonate (315 mg, 2.28 mmol) and sodium iodide (3.45 mg, 0.023 mmol) in acetone (10 ml) was stirred at 60° C. for 18 hours. The solid was filtered and washed with acetone. The resulting filtrate was concentrated and the resulting product was purified by column chromatography utilizing an ISCO system (0.1% triethyl amine and 5% methanol in DCM) to give 45 mg (38.8%) of a white solid. NMR (400 MHz): δ 10.75 (s, 1H), 8.35 (m, 2H), 8.25 (s, 1H), 8.05 (d, 1H), 7.80-7.95 (m, 4H), 7.70 (s, 1H), 7.62 (m, 1H), 7.50 (d, 1H), 4.40 (t, 2H), 3.22 (t, 2H), 2.65 (s, 6H), 2.10 (s, 3H); m/z 511.

### Examples 21-26

The following examples were synthesised by the procedure of Example 20 using N-[3-(6-hydroxy-4-oxo-4H-quinazolin-3-yl)-4-methylphenyl]-3-trifluoromethylbenzamide (Example 52) and the appropriate chloro compound as starting materials.

| Ex | Compound | NMR | m/z |
|---|---|---|---|
| 21 | N-{3-[6-(3-Dimethylamino- | 10.75 (s, 1H), 8.38 (s, 1H), 8.32 (d, 1H), | 525 |
| | propoxy)-4-oxo-4H- | 8.26 (s, 1H), 8.07 (d, 1H), 7.95 (m, 3H), 7.82 (d, | |
| | quinazolin-3-yl]-4- | 1H), 7.70 (s, 1H), 7.55 (m, 2H), 4.25 (m, 2H), | |
| | methylphenyl}-3- | 2.75 (m, 2H), 2.42 (s, 6H), 2.15 (s, 3H), | |
| | trifluoromethylbenzamide | 2.05 (m, 2H) | |
| 22 | N-{4-Methyl-3-[6-(2- | 10.56 (s, 1H), 8.16 (m, 3H), 7.90 (d, 1H), | 553 |
| | morpholinoethoxy)-4-oxo- | 7.80 (s, 1H), 7.70 (m, 3H), 7.60 (s, 1H), 7.45 (d, | |
| | 4H-quinazolin-3-yl]phenyl}- | 1H), 7.35 (d, 1H), 4.40 (m, 2H), 3.40-3.60 (m, | |
| | 3-trifluoromethylbenzamide | 8H), 3.12 (m, 2H), 1.96 (s, 3H) | |
| 23 | N-{4-Methyl-3-[4-oxo-6-(2- | 10.70 (s, 1H), 8.35 (m, 3H), 8.05 (d, 1H), | 551 |
| | piperidin-1-ylethoxy)-4H- | 7.95 (s, 1H), 7.86 (m, 3H), 7.75 (s, 1H), 7.62 (d, | |
| | quinazolin-3-yl]phenyl}-3- | 1H), 7.52 (d, 1H), 4.55 (m, 2H), 3.55 (m, 4H), | |
| | trifluoromethylbenzamide | 3.10 (m, 2H), 2.15 (s, 3H), 1.92 (m, 2H), | |
| | | 1.75 (m, 3H), 1.50 (m, 1H) | |
| 24 | N-{3-[6-(2-Diethylamino- | 10.70 (s, 1H), 8.30 (m, 3H), 8.00 (d, 1H), | 539 |
| | ethoxy)-4-oxo-4H- | 7.95 (s, 1H), 7.80 (m, 3H), 7.70 (s, 1H), 7.60 (d, | |
| | quinazolin-3-yl]-4- | 1H), 7.50 (d, 1H), 4.47 (t, 2H), 3.62 (t, 2H), | |
| | methylphenyl}-3- | 3.30 (m, 4H), 2.10 (s, 3H), 1.30 (t, 6H) | |
| | trifluoromethylbenzamide | | |
| 25 | N-{4-Methyl-3-[4-oxo-6-(2- | 10.40 (s, 1H), 7.92-8.10 (m, 3H), | 537 |
| | pyrrolidin-1-yl-ethoxy)-4H- | 7.50-7.72 (m, 5H), 7.20-7.40 (m, 3H), 4.00 (m, 2H), | |
| | quinazolin-3-yl]phenyl}-3- | 2.60 (m, 2H), 2.30 (m, 4H), 1.85 (s, 3H), | |
| | trifluoromethylbenzamide | 1.42 (m, 4H) | |
| 26 | N-[4-Methyl-3-(6- | 10.57 (s, 1H), 8.20 (m, 3H), 7.92 (d, 1H), | 496 |
| | oxiranylmethoxy-4-oxo-4H- | 7.70 (m, 4H), 7.55 (s, 1H), 7.50 (d, 1H), 7.40 (d, | |
| | quinazolin-3-yl)phenyl]-3- | 1h), 4.50 (m, 1H), 3.92 (m, 1H), 2.85 (m, 1H), | |
| | trifluoromethylbenzamide | 2.75 (m, 1H), 2.50 (m, 1H), 2.00 (s, 3H) | |

### Example 27

### N-[3-(6-Bromo-4-oxo-4H-quinazolin-3-yl)-4-methylphenyl]-3-(1-cyano-1-methylethyl)benzamide

A mixture of 3-(5-amino-2-methylphenyl)-6-bromo-3H-quinazolin-4-one (Method 18; 2g, 6.06 mmol), 3-(1-cyano-1-methylethyl)benzoic acid (Method 11; 1.15 g, 6.06 mmol), EDCI (2.3 g, 12.12 mmol), HOBt (818 mg, 6.06 mmol) and diisopropyl ethyl amine (1.17 g, 9.09 mmol, 1.5 eq) in DMF (20 ml) were stirred at 25° C. for 72 hours. The reaction mixture was diluted with DCM, washed with water, brine and dried with Na₂SO₄ (s). The solvents were removed under reduced pressure to afford an oil that was purified by column chromatography utilizing an ISCO system (hexane-EtOAc) to give 1.61 g (53%) of a white solid. NMR (400 MHz): 10.55 (s, 1H), 7.55-8.50 (m, 11H), 2.15 (s, 3H), 1.80 (s, 6H); m/z 502.

### Examples 28-46

The following compounds were synthesized as described in Example 27 from 3-(5-amino-2-methylphenyl)-8-methoxyquinazolin-4(3H)-one (Method 39) and the appropriate carboxylic acid.

| Ex | Compound | NMR | m/z | S.M |
|---|---|---|---|---|
| 28 | N-[3-(8-Methoxy-4- | 10.59 (s, 1H), 8.90 (s, 1H), | 400 | 5- |
| | oxoquinazolin-3(4H)-yl)- | 8.60 (s, 1H), 8.24 (s, 1H), | | Methylpyridine- |
| | 4-methylphenyl]-5- | 8.11 (s, 1H), 7.83 (m, 1H), 7.77 (dd, | | 3-carboxylic acid |
| | methylnicotinamide | 2H), 7.50-7.60 (m, 1H), | | |
| | | 7.41-7.49 (m, 2H), 3.93 (s, 3H), | | |
| | | 2.37 (s, 3H), 2.05 (s, 3H) | | |
| 29 | N-[3-(8-Methoxy-4- | 10.60 (s, 1H), 8.26 (s, 1H), | 400 | 6- |
| | oxoquinazolin-3(4H)-yl)- | 7.89-8.00 (m, 4H), 7.76 (d, | | Methylpyridine- |
| | 4-methylphenyl]-6- | 1H), 7.42-7.57 (m, 4H), | | 2-carboxylic acid |
| | methylpyridine-2- | 3.94 (s, 3H), 2.61 (s, 3H), 2.06 (s, | | |
| | carboxamide | 3H) | | |
| 30 | 4-Methoxy-N-[3-(8- | 10.58 (s, 1H), 8.24-8.34 (m, | 483 | 4-Methoxy-3- |
| | methoxy-4- | 3H), 7.82-7.87 (m, 2H), | | (trifluoromethyl)benzoic |
| | oxoquinazolin-3(4H)-yl)- | 7.75 (d, 1H), 7.54 (t, 1H), 7.44 (m, | | acid |
| | 4-methylphenyl]-3- | 3H), 3.97 (s, 3H), 3.94 (s, 3H), | | |
| | (trifluoromethyl)- | 2.05 (s, 3H) | | |
| | benzamide | | | |
| 31 | N-[3-(8-Methoxy-4- | 10.67 (s, 1H), 8.24 (s, 1H), | 467 | 2-methyl-5- |
| | oxoquinazolin-3(4H)-yl)- | 7.80 (s, 2H), 7.71-7.77 (m, | | (trifluoromethyl)benzoic |
| | 4-methylphenyl]-2- | 3H), 7.54 (m, 2H), | | acid |
| | methyl-5- | 7.40-7.47 (m, 2H), 3.93 (s, 3H), 2.45 (s, | | |
| | (trifluoromethyl)- | 3H), 2.04 (s, 3H) | | |
| | benzamide | | | |
| 32 | 2-Chloro-N-[3-(8- | 10.86 (s, 1H), 8.24 (s, 1H), | 488 | 2-chloro-5- |
| | methoxy-4- | 8.04 (s, 1H), 7.88 (m, 1H), | | (trifluoromethyl)benzoic |
| | oxoquinazolin-3(4H)-yl)- | 7.68-7.85 (m, 4H), 7.53 (m, | | acid |
| | 4-methylphenyl]-5- | 1H), 7.42-7.49 (m, 2H), | | |
| | (trifluoromethyl)- | 3.94 (s, 3H), 2.05 (s, 3H) | | |
| | benzamide | | | |
| 33 | 2-Fluoro-N-[3-(8- | 10.80 (s, 1H), 8.24 (s, 1H), | 471 | 2-fluoro-5- |
| | methoxy-4- | 8.06 (d, 2H), 7.95-8.03 (m, | | (trifluoromethyl)benzoic |
| | oxoquinazolin-3(4H)-yl)- | 1H), 7.69-7.80 (m, 3H), | | acid |
| | 4-methylphenyl]-5- | 7.51-7.65 (m, 2H), 7.43-7.47 (m, | | |
| | (trifluoromethyl)- | 2H), 3.94 (s, 3H), 2.05 (s, 3H) | | |
| | benzamide | | | |
| 34 | 3-Fluoro-N-[3-(8- | 10.71 (s, 1H), 8.24 (s, 1H), | 471 | 3-fluoro-5- |
| | methoxy-4- | 8.17 (s, 1H), 8.12 (d, 1H), | | (trifluoromethyl)benzoic |
| | oxoquinazolin-3(4H)-yl)- | 7.98 (d, 1H), 7.72-7.85 (m, 3H), | | acid |
| | 4-methylphenyl]-5- | 7.44-7.57 (m, 3H), 3.94 (s, | | |
| | (trifluoromethyl)- | 3H), 2.06 (s, 3H) | | |
| | benzamide | | | |
| 35 | 4-Fluoro-N-[3-(8- | 10.57 (s, 1H), 8.26 (m, 2H), | 471 | 4-fluoro-3- |
| | methoxy-4- | 8.14 (s, 1H), 7.69-7.77 (m, | | (trifluoromethyl)benzoic |
| | oxoquinazolin-3(4H)-yl)- | 2H), 7.57-7.68 (m, 2H), | | acid |
| | 4-methylphenyl]-3- | 7.40-7.50 (m, 1H), 7.33-7.39 (m, | | |
| | (trifluoromethyl)- | 2H), 3.84 (s, 3H), 1.96 (s, 3H) | | |
| | benzamide | | | |
| 36 | N-[3-(8-Methoxy-4- | 10.72 (s, 1H), 8.23 (s, 1H), | 457 | 1-methyl-3- |
| | oxoquinazolin-3(4H)-yl)- | 7.72-7.85 (m, 3H), | | (trifluoromethyl)- |
| | 4-methylphenyl]-1- | 7.49-7.63 (m, 2H), 7.41-7.49 (m, 2H), | | 1H-pyrazole-5- |
| | methyl-3- | 4.15 (s, 3H), 3.94 (s, 3H), | | carboxylic acid |
| | (trifluoromethyl)-1H- | 2.05 (s, 3H) | | |
| | pyrazole-5-carboxamide | | | |
| 37 | N-[3-(8-Methoxy-4- | 10.27 (s, 1H), 8.33 (s, 1H), | 478 | 3- |
| | oxoquinazolin-3(4H)-yl)- | 8.25 (s, 1H), 7.65-7.87 (m, | | [(methylsulfonyl)- |
| | 4-methylphenyl]-3- | 5H), 7.35-7.60 (m, 2H), | | amino]benzoic |
| | [(methylsulfonyl)amino]- | 5.75 (s, 1H), 3.94 (s, 3H), 2.04 (s, | | acid |
| | benzamide | 3H), 1.78 (s, 7H), 1.08 (s, 1H) | | |
| 38 | 3-tert-Butyl-N-[3-(8- | 10.43 (s, 1H), 8.24 (s, 1H), | 442 | Method 21 |
| | methoxy-4- | 9.20 (s, 1H), 7.94-7.83 (m, | | |
| | oxoquinazolin-3(4H)-yl)- | 3H), 7.77 (t, 1H), 7.62 (d, 1H), | | |
| | 4-methylphenyl]- | 7.55 (t, 1H), 7.46-7.41 (m, | | |
| | benzamide | 3H), 3.94 (s, 3H), 2.05 (s, 3H), | | |
| | | 1.30 (s, 9H) | | |
| 39 | 2-(1-Cyano-1- | 10.81 (s, 1H), 8.80 (d, 1H), | 454 | Method 20 |
| | methylethyl)-N-[3-(8- | 8.24 (s, 1H), 8.03 (s, 1H), | | |
| | methoxy-4- | 7.86-7.82 (m, 3H), 7.75 (d, | | |
| | oxoquinazolin-3(4H)-yl)- | 1H), 7.54 (t, 1H), | | |
| | 4-methylphenyl]- | 7.46-7.44 (m, 2H), 3.94 (s, 3H), 2.06 (s, | | |
| | isonicotinamide | 3H), 1.76 (s, 6H) | | |
| 40 | 4-Chloro-3-(1-cyano-1- | 10.77 (s, 1H), 8.24 (s, 1H), | 488 | Method 12 |
| | methylethyl)-N-[3-(8- | 8.05 (d, 1H), 7.99 (dd, 1H), | | |
| | methoxy-4- | 7.87-7.85 (m, 2H), | | |
| | oxoquinazolin-3(4H)-yl)- | 7.76-7.71 (m, 3H), 7.54 (t, 1H), 7.44 (t, | | |
| | 4-methylphenyl]- | 1H), 3.94 (s, 3H), 2.05 (s, 3H), | | |
| | benzamide | 1.86 (s, 6H) | | |
| 41 | 5-(1-Cyano-1- | 10.63 (s, 1H), 8.23 (s, 1H), | 459 | Method 30 |
| | methylethyl)-N-[3-(8- | 8.05 (d, 1H), 7.83-7.81 (m, | | |
| | methoxy-4- | 2H), 7.75 (dd, 1H), 7.54 (t, | | |
| | oxoquinazolin-3(4H)-yl)- | 1H), 7.46-7.43 (m, 2H), | | |
| | 4-methylphenyl]- | 7.29 (d, 1H), 3.94 (s, 3H), 2.04 (s, | | |
| | thiophene-2-carboxamide | 3H), 1.78 (s, 6H) | | |
| 42 | 5-(1-Cyano-1- | 10.35 (s, 1H), 8.24 (s, 1H), | 459 | Method 31 |
| | methylethyl)-N-[3-(8- | 7.84-7.71 (m, 5H), 7.54 (t, | | |
| | methoxy-4- | 1H), 7.46-7.41 (m, 2H), | | |
| | oxoquinazolin-3(4H)-yl)- | 3.94 (s, 3H), 2.04 (s, 3H), 1.78 (s, | | |
| | 4-methylphenyl]- | 6H) | | |
| | thiophene-3-carboxamide | | | |
| 43 | N-[3-(8-Methoxy-4- | 10.83 (s, 1H), 8.51 (m, 1H), | 464 | 3- |
| | oxoquinazolin-3(4H)-yl)- | 8.31 (d, 1H), 8.25 (s, 1H), | | (methylsulfonyl)- |
| | 4-methylphenyl]-3- | 8.13 (m, 1H), 7.77-7.88 (m, 3H), | | benzoic acid |
| | (methylsulfonyl)- | 7.75 (dd, 1H), 7.54 (t, 1H), | | |
| | benzamide | 7.45 (m, 2H), 3.94 (s, 3H), | | |
| | | 3.33 (s, 3H), 2.06 (s, 3H) | | |
| 44 | N-[3-(8-Methoxy-4- | 10.73 (s, 1H), 8.29 (m, 2H), | 535 | Method 32 |
| | oxoquinazolin-3(4H)-yl)- | 8.24 (s, 1H), 7.94 (m, 1H), | | |
| | 4-methylphenyl]-3- | 7.84 (m, 3H), 7.76 (dd, 1H), | | |
| | (morpholin-4- | 7.54 (t, 1H), 7.45 (d, 2H), | | |
| | ylsulfonyl)benzamide | 3.94 (s, 3H), 3.63 (m, 4H), 2.90 (m, | | |
| | | 4H), 2.06 (s, 3H) | | |
| 45 | 3-(Azetidin-1- | 10.75 (s, 1H), 8.34 (m, 2H), | 505 | Method 33 |
| | ylsulfonyl)-N-[3-(8- | 8.25 (s, 1H), 8.01 (m, 1H), | | |
| | methoxy-4- | 7.85 (m, 3H), 7.75 (dd, 1H), | | |
| | oxoquinazolin-3(4H)-yl)- | 7.54 (t, 1H), 7.45 (d, 2H), | | |
| | 4-methylphenyl]- | 3.94 (s, 3H), 3.70 (t, 4H), 2.06 (s, | | |
| | benzamide | 3H), 1.99 (m, 2H) | | |
| 46 | 3-[(Cyclopropylamino)- | 10.82 (s, 1H), 8.42 (m, 1H), | 505 | Method 34 |
| | sulfonyl]-N-[3-(8- | 8.28 (m, 1H), 8.24 (s, 1H), | | |
| | methoxy-4- | 8.08 (m, 1H), 8.00 (m, 1H), | | |
| | oxoquinazolin-3(4H)-yl)- | 7.88 (m, 1H), 7.76 (m, 2H), | | |
| | 4-methylphenyl]- | 7.54 (t, 1H), 7.45 (m, 2H), | | |
| | benzamide | 3.94 (s, 3H), 2.12 (m, 1H), 2.06 (s, | | |
| | | 3H), 0.46 (m, 2H), 0.34 (m, | | |
| | | 2H) | | |

### Example 47

### 3-(1-Cyano-1-methylethyl)-N-[4-methyl-3-(6-morpholino-4-oxo-4H-quinazolin-3-yl)phenyl]benzamide

A microwave vial was charged with sodium tert-butoxide (29 mg, 0.24 mmol), Pd₂(dba)₃ (15 mg, 10% mmol), BINAP (20 mg, 20% mmol) and N-[3-(6-bromo-4-oxo-4H-quinazolin-3-yl)-4-methylphenyl]-3-(1-cyano-1-methylethyl)benzamide (Example 27; 80 mg, 0.16 mmol). The vial was fitted with a septum and purged with nitrogen. 1,4-Dioxane (3.3 ml) and morpholine (33 mg, 0.38 mmol, 2.4 eq) were then added via syringe. The vial was irradiated in a microwave at 175° C. for 30 min. The mixture was filtered through a pad of silica gel and washed with DCM. The filtrate was concentrated and purified first by column chromatography utilizing an ISCO system (0.5% triethyl amine, 5% methanol in DCM) and then by reverse phase chromatography utilizing a Gilson HPLC (0.1% TFA in acetonitrile-water) to give 25 mg (30.9%) of a white solid. NMR (400 MHz): 10.31 (s, 1H), 8.00 (s, 1H), 7.90 (s, 1H), 7.80 (d, 1H), 7.30-7.69 (m, 8H), 3.65 (t, 4H), 3.15 (t, 4H), 1.95 (s, 3H), 1.60 (s, 6H); m/z 508.

### Example 48

The following compound was prepared according to Example 47 using N-[3-(6-bromo-4-oxo-4H-quinazolin-3-yl)-4-methylphenyl]-3-(1-cyano-1-methylethyl)benzamide (Example 27) and the appropriate amine as starting materials.

| Ex | Compound | NMR | m/z |
|---|---|---|---|
| 48 | 3-(1-Cyano-1-methylethyl)- | 10.22 (s, 1H), 7.85 (s, 1H), 7.80 (s, 1H), | 523 |
| | N-{3-[6-(3-dimethylamino- | 7.70 (d, 1H), 7.45-7.60 (m, 5H), | |
| | propylamino)-4-oxo-4H- | 7.10-7.40 (m, 5H), 2.85 (m, 2H), 2.60 (m, 2H), | |
| | quinazolin-3-yl]-4- | 1.85 (s, 6H), 1.55 (s, 6H), 1.50 (m, 2H) | |
| | methylphenyl}benzamide | | |

### Example 49

### 3-(1-Cyano-1-methylethyl)-N-{3-[6-(4-ethylpiperazin-1-yl)-4-oxo-4H-quinazolin-3-yl]-4-methylphenyl}benzamide

A microwave vial was charged with caesium carbonate (194 mg, 0.599 mmol), Pd₂(dba)₃ (36.5 mg, 10% mmol), tri-t-butyl phosphine (10% wt in hexane, 160 μl, 20% mmol) and N-[3-(6-bromo-4-oxo-4H-quinazolin-3-yl)-4-methylphenyl]-3-(1-cyano-1-methylethyl)benzamide (Example 27; 200 mg, 0.399 mmol). The vial was fitted with a septum and purged with nitrogen. 1,4-Dioxane and 1-ethylpiperizine (91 mg, 0.798 mmol, 2.0 eq) were then added via syringe. The vial was irradiated in a microwave at 165° C. for 20 min. The mixture was filtered through a pad of silica gel and washed with DCM. The filtrate was concentrated and then purified by column chromatography utilizing an ISCO system (0.2 triethyl amine, 5% methanol in DCM) to give 110 mg (51.6%) of light a yellow solid. NMR (400 MHz): 10.58 (s, 1H), 8.20 (s, 1H), 8.04 (s, 1H), 7.95 (d, 1H), 7.35 (m, 2H), 7.23 (m, 3H), 7.60 (m, 2H), 7.45 (d, 1H), 4.01 (m, 2H), 3.60 (m, 2H), 3.20 (m, 6H), 2.10 (s, 3H), 1.73 (s, 6H), 1.30 (t, 3H); m/z 535.

### Example 50

The following compound was prepared according to Example 49 using N-[3-(7-bromo-4-oxo-4H-quinazolin-3-yl)-4-methylphenyl]-3-(1-cyano-1-methylethyl)benzamide (Example 51) as the starting material.

| Ex | Compound | NMR | m/z |
|---|---|---|---|
| 50 | 3-(1-Cyano-1-methylethyl)- | 10.52 (s, 1H), 8.20 (s, 1H), 8.10 (s, 1H), | 523 |
| | N-{3-[7-(3-dimethylamino- | 8.01 (d, 1H), 8.00 (d, 1H), 7.80 (m, 3H), | |
| | propylamino)-4-oxo-4H- | 7.70 (m, 1H), 7.49 (d, 1H), 7.05 (d, 1h), | |
| | quinazolin-3-yl]-4- | 6.90 (s, 1H), 6.75 (br, 1H), 3.18 (m, 2H), | |
| | methylphenyl}benzamide | 2.90 (m, 2H), 2.55 (m, 2H), 2.10 (s, 3H), 1.80 | |
| | | (s, 6H), 1.05 (s, 6H) | |

### Example 51

### N-[3-(7-Bromo-4-oxo-4H-quinazolin-3-yl)-4-methylphenyl]-3-(1-cyano-1-methylethyl)benzamide

A stirred mixture of 2-amino-4-bromobenzoic acid (Method 3; 273 mg, 1.26 mmol), triethyl orthoformate (280 mg, 310 μl, 1.89 mmol) and acetic acid (7 μl, 0.13 mmol) in toluene (8 ml) was heated at reflux for 2.5 hours. N-(3-Amino-4-methylphenyl)-3-(1-cyano-1-methylethyl)benzamide (Method 15; 370 mg, 1.26 mmol) was then added to the mixture and stirred at 120° C. for 32 hours. The solvents were removed under reduced pressure and the resulting product was purified by column chromatography utilizing an ISCO system (hexane-EtOAc) to yield 131 mg (20.8%) of a white solid. NMR (400 MHz): 10.50 (s, 1H), 8.40 (s, 1H), 8.20 (d, 1H), 8.08 (m, 2H), 8.00 (d, 1H), 7.76-7.90 (m, 4H), 7.65 (m, 1H), 7.49 (d, 1H), 2.12 (s, 3H), 1.80 (s, 6H); m/z 502.

### Example 52

### N-[3-(6-Hydroxy-4-oxo-4H-quinazolin-3-yl)-4-methylphenyl]-3-trifluoromethylbenzamide

N-[3-(6-Methoxy-4-oxo-4H-quinazolin-3-yl)-4-methylphenyl]-3-trifluoromethyl-benzamide (Example 19; 1.8 g, 4.0 mmol) and BBr₃ (10 ml of 1.0 M solution in DCM) were stirred in DCM (10 ml) for 20 hours. The reaction was quenched with water and then diluted with 2 M NaOH. The aqueous layer was washed with DCM (10 ml) and then acidified with 2 M HCl and extracted with EtOAc (10 ml). The combined organics were dried with Na₂SO₄ (s) and concentrated under reduced pressure to give a white solid (1.4 g, 85% crude). NMR (400 MHz): δ 10.8 (s, 1H), 7.3-8.2 (m, 11H), 3.3 (brs, 1H); m/z: 440.

### Example 53

### N-(3-{7-[1-(t-Butoxycarbonyl)azetidin-3-ylamino]-4-oxo-4H-quinazolin-3-yl}-4-methylphenyl)-3-(1-cyano-1-methylethyl)benzamide

A microwave vial was charged with sodium tert-butoxide (33 mg, 0.299 mmol), Pd₂(dba)₃ (18 mg, 10% mmol), BINAP (24 mg, 20% mmol) and N-[3-(7-bromo-4-oxo-4H-quinazolin-3-yl)-4-methylphenyl]-3-(1-cyano-1-methylethyl)benzamide (Example 51; 100 mg, 0.199 mmol). The vial was fitted with a septum and purged with nitrogen. 3-Amino-azetidine-1-carboxylic acid tert-butyl ester (82 mg, 0.478 mmol, 2.4 eq) in dioxane was added dropwise via a syringe. The vial was irradiated in a microwave at 175° C. for 30 min. The mixture was then filtered through a pad of silica gel and washed with DCM. The filtrate was concentrated and then purified by column chromatography utilizing an ISCO system (hexane-EtOAc) to give 80 mg (67.9%) of a yellow solid. NMR (400 MHz): 10.25 (s, 1H), 7.96 (s, 1H), 7.85 (s, 1H), 7.72 (m, 2H), 7.60 (d, 1H), 7.56 (m, 2H), 7.41 (m, 1H), 7.25 (d, 1H), 7.18 (d, 1H), 6.65 (d, 1H), 6.40 (s, 1H), 4.10 (m, 3H), 3.51 (m, 2H), 1.87 (s, 3H), 1.53 (s, 6H), 1.21 (s, 9H); m/z 593.

### Example 54

The following compound was prepared according to Example 53 using the starting material illustrated and the appropriate amine.

| Ex | Compound | NMR | m/z | SM |
|---|---|---|---|---|
| 54 | 3-(1-Cyano-1-methylethyl)- | 10.30 (s, 1H), 8.05 (s, 1H), 7.89 (s, | 509 | Example |
| | N-{4-methyl-3-[7-(N- | 1H), 7.81 (m, 1H), 7.75 (m, 3H), | | 51 |
| | methylcarbamoylmethyl | 7.62 (m, 3H), 7.45 (m, 1H), 7.25 (d, | | |
| | amino)-4-oxo-4H- | 1H), 6.75 (dd, 1H), 6.40 (s, 1H), | | |
| | quinazolin-3-yl]- | 3.65 (s, 2H), 2.48 (d, 3H), 1.90 (s, | | |
| | phenyl}benzamide | 3H), 1.55 (s, 6H) | | |
| 55 | N-(3-{6-[1-(t- | | 593 | Example |
| | Butoxycarbonyl)azetidin-3- | | | 27 |
| | ylamino]-4-oxo-4H- | | | |
| | quinazolin-3-yl}-4- | | | |
| | methylphenyl)-3-(1-cyano-1- | | | |
| | methylethyl)benzamide | | | |

### Example 56

N-{3-[7-(Azetidin-3-ylamino)-4-oxo-4H-quinazolin-3-yl]-4-methylphenyl}-3-(1-cyano-1-methylethyl)benzamide

N-(3-{7-[1-(t-Butoxycarbonyl)azetidin-3-ylamino]-4-oxo-4H-quinazolin-3-yl}-4-methylphenyl)-3-(1-cyano-1-methylethyl)benzamide (Example 53; 79 mg, 0.133 mmol) was treated with 4 M HCl in dioxane. The mixture was stirred at 25° C. for 2 hours. The suspension was diluted with diethyl ether (4 ml) and stirred for 30 min. The light yellow solid (65 mg, 100%) was collected by filtration, washed with ethyl ether, and dried. NMR (400 MHz): 10.61 (s, 1H), 9.33 (br, 2H), 8.42 (s, 1H), 8.15 (s, 1H), 8.05 (m, 2H), 7.90 (m, 2H), 7.80 (d, 1H), 7.65 (m, 1H), 7.50 (d, 1H), 6.95 (d, 1H), 6.75 (s, 1H), 4.63 (m, 1H), 4.42 (m, 2H), 4.00 (m, 2H), 2.12 (s, 3H), 1.80 (s, 6H); m/z 493.

### Example 57

The following compound was prepared according to Example 56 using N-(3-{6-[1-(t-butoxycarbonyl)azetidin-3-ylamino]-4-oxo-4H-quinazolin-3-yl}-4-methylphenyl)-3-(1-cyano-1-methylethyl)benzamide (Example 55) as the starting material.

| Ex | Compound | NMR | m/z |
|---|---|---|---|
| 57 | N-{3-[6-(Azetidin-3- | 10.60 (s, 1H), 9.00 (br, 3H), 8.20 (m, 2H), | 494 |
| | ylamino)-4-oxo-4H- | 8.10 (s, 1H), 8.00 (d, 1H), 7.80 (m, 3H), | |
| | quinazolin-3-yl]-4- | 7.55 (d, 1H), 7.40 (d, 1H), 7.15 (d, 1H), | |
| | methylphenyl}-3- | 7.05 (s, 1H), 4.45 (m, 1H), 4.20 (m, 2H), 3.80 | |
| | trifluoromethylbenzamide | (m, 2H), 2.00 (s, 3H) | |

### Example 58

### 3-(1-Cyano-1-methylethyl)-N-[4-methyl-3-(4-oxo-7-pyrimidin-5-yl-4H-quinazolin-3-yl)phenyl]-benzamide

A microwave vial was charged with caesium carbonate (259 mg, 0.796 mmol), Pd(PPh₃)₄ (17 mg, 7.5% mmol), 5-pyrimidine boronic acid (30 mg, 0.239 mmol) and N-[3-(7-bromo-4-oxo-4H-quinazolin-3-yl)-4-methylphenyl]-3-(1-cyano-1-methylethyl)benzamide (Example 51; 100 mg, 0.199 mmol). The vial was fitted with a septum and purged with nitrogen. 1,4-Dioxane and water (4:1) (3 ml) was then added via syringe. The vial was irradiated in a microwave at 165° C. for 20 min. The mixture was then filtered through a pad of silica gel and washed with DCM. The filtrate was concentrated and purified first by column chromatography utilizing an ISCO system (0.5% triethyl amine, 5% methanol in DCM) and then by reverse phase chromatography utilizing a Gilson HPLC (0.1% TFA in acetonitrile-water) to give 20 mg (20%) of a light yellow solid. NMR (400 MHz): 10.45 (s, 1H), 9.26 (s, 2H), 9.21 (s, 1H), 8.32 (s, 1H), 8.30 (d, 1H), 8.20 (s, 1H), 8.00 (m, 2H), 7.90 (d, 1H), 7.80 (m, 2H), 7.70 (d, 1H), 7.55 (m, 1H), 7.40 (d, 1H), 2.00 (s, 3H), 1.59 (s, 6H); m/z 501.

### Examples 59-61

The following compounds were synthesized according to Example 58.

| Ex | Compound | NMR | m/z | S.M |
|---|---|---|---|---|
| 59 | 3-(1-Cyano-1- | 10.26 (s, 1H), 8.15 (m, 2H), | 489 | 4-(4,4,5,5- |
| | methylethyl)-N-{4- | 7.98 (d, 1H), 7.81 (m, 2H), 7.75 (m, | | tetramethyl[1,3,2]- |
| | methyl-3-[4-oxo-7- | 2H), 7.60 (m, 1H), 7.55 (d, 1H), | | dioxaborolan-2- |
| | (1H-pyrazol-4-yl)-4H- | 7.25-7.40 (m, 5H), 1.90 (s, 3H), | | yl)-1H-pyrazole |
| | quinazolin-3-yl]- | 1.53 (s, 6H) | | and Example 51 |
| | phenyl}benzamide | | | |
| 60 | 3-(1-Cyano-1- | 11.91 (s, 1H), 10.71 (s, 1H), | 488 | 2-boronic acid- |
| | methylethyl)-N-{4- | 8.50 (s, 1H), 8.37 (d, 1H), 8.29 (s, | | pyrrole-1- |
| | methyl-3-[4-oxo-7- | 1H), 8.22 (s, 1H), 8.20 (d, 1H), | | carboxylic acid |
| | (1H-pyrrol-2-yl)-4H- | 8.10 (d, 1H), 8.02 (m, 1H), | | tert-butyl ester |
| | quinazolin-3-yl]- | 7.95 (d, 1H), 7.85 (m, 2H), 7.65 (d, | | and Example 51 |
| | phenyl}benzamide | 1H), 7.22 (s, 1H), 7.05 (s, 1H), | | |
| | | 6.40 (s, 1H), 2.30 (s, 3H), | | |
| | | 1.95 (s, 6H) | | |
| 61 | N-[4-Methyl-3-(4-oxo- | 10.40 (s, 1H), 8.95 (s, 1H), | 501 | 3-pyrimidine |
| | 7-pyridin-3-yl-4H- | 8.50 (d, 1H), 8.30 (d, 1H), 8.16 (s, | | boronic acid and |
| | quinazolin-3- | 1H), 8.10 (d, 1H), 8.05 (s, 1H), | | Example 16 |
| | yl)phenyl]-3- | 8.01 (d, 1H), 7.95 (d, 1H), | | |
| | trifluoromethyl- | 7.80 (d, 1H), 7.72 (d, 1H), 7.67 (s, | | |
| | benzamide | 1H), 7.60 (m, 3H), 7.25 (d, 1H), | | |
| | | 1.90 (s, 3H) | | |

### Example 62

### 3-(1-Cyano-1-methylethyl-N-[3-(8-methoxy-4-oxo-4H-quinazolin-3-yl)-4-methylphenyl]-benzamide

A suspension of 8-methoxybenzo[d][1,3]oxazin-4-one (157 mg, 0.887 mmol) and N-(3-amino-4-methylphenyl)-3-(1-cyano-1-methylethyl)benzamide (Method 15; 260 mg, 0.887 mmol) in anhydrous toluene (5 ml) was heated to reflux for 25 h. The solid was filtered off and washed with methanol and DCM. The filtrate was concentrated and the resulting product was purified by column chromatography utilizing an ISCO system (hexane-EtOAc) to give 65 mg (16.2%) of a white solid. NMR (400 MHz): 10.55 (s, 1H), 8.30 (s, 1H), 8.10 (s, 1H), 8.00 (d, 1H), 7.90 (m, 2H), 7.81 (m, 2H), 7.60-7.65 (m, 2H), 7.50 (m, 2H), 4.00 (s, 3H), 2.10 (s, 3H), 1.80 (s, 6H); m/z 453.

### Example 63

### 3-(1-Cyano-1-methylethyl)-N-[3-(8-hydroxy-4-oxo-4H-quinazolin-3-yl)-4-methylphenyl]-benzamide

3-(1-Cyano-1-methylethyl)-N-[3-(8-methoxy-4-oxo-4H-quinazolin-3-yl)-4-methylphenyl]-benzamide (Example 62; 45 mg, 0.1 mmol) was suspended in 1 M BBr₃ in DCM (2 ml). The mixture was stirred at 25° C. for 2 hours and then quenched with methanol. The solvents were removed under reduced pressure and the resulting product was purified by reverse phase chromatography utilizing a Gilson HPLC (0.1% TFA in acetonitrile-water) to give 40 mg (91.8%) of a green solid. NMR (400 MHz): 10.53 (s, 1H), 8.32 (s, 1H), 8.11 (s, 1H), 8.00 (d, 1H), 7.90 (m, 2H), 7.80 (d, 1H), 7.60 (m, 2H), 7.40 (m, 2H), 7.30 (d, 1H), 2.12 (s, 3H), 1.80 (s, 6H); m/z 439.

### Example 64

### N-[3-(8-Chloro-4-oxo-4H-quinazolin-3-yl)-4-methylphenyl]-3-(1-cyano-1-methylethyl)benzamide

A stirred mixture of 2-amino-3-chloro-benzoic acid (2.5 g, 14.6 mmol), triethyl orthoformate (15 ml) and acetic acid (0.5 ml) in toluene (20 ml) was heated under reflux for 4 hours. N-(3-Amino-4-methylphenyl)-3-(1-cyano-1-methylethyl)benzamide (Method 15; 2.53 g, 8.6 mmol) was added to the mixture and stirred under reflux for 16 hours. The product was collected by filtration to yield 2.5 g (63.8%) of a white solid. NMR (400 MHz): 10.58 (s, 1H) 8.50 (s, 1H), 8.25 (d, 1H), 8.10 (m, 2H), 8.00 (d, 1H), 7.90 (m, 2H), 7.80 (d, 1H), 7.65 (m, 2H), 7.50 (d, 1H), 2.13 (s, 3H), 1.80 (s, 6H); m/z 457.

### Example 65

### 3-(1-Cyano-1-methylethyl)-N-{4-methyl-3-[8-(N-methylcarbamoylmethylamino)-4-oxo-4H-quinazolin-3-yl]-phenyl}-benzamide

A microwave vial was charged with sodium tert-butoxide (60 mg, 0.493 mmol), Pd₂(dba)₃ (18 mg, 10% mmol), BINAP (24 mg, 20% mmol), N-[3-(8-chloro-4-oxo-4H-quinazolin-3-yl)-4-methylphenyl]-3-(1-cyano-1-methylethyl)benzamide (Example 64; 90 mg, 0.197 mmol) and H-Gly-NHMe hydrochloride (58.9 mg, 0.474 mmol). The vial was fitted with a septum and purged with nitrogen. 1,4-Dioxane (3 ml) was then added via syringe. The vial was irradiated in a microwave at 175° C. for 30 min. The mixture was then filtered through a pad of silica gel and washed with DCM. The filtrate was concentrated and purified first by column chromatography utilizing an ISCO system (0.5% triethyl amine, 5% methanol in DCM) and then by reverse phase chromatography utilizing a Gilson HPLC (0.1% TFA in acetonitrile-water) to give 35 mg (35%) of a white solid. NMR (400 MHz): 10.30 (s, 1H), 8.03 (s, 1H), 7.89 (s, 1H), 7.70-7.81 (m, 3H), 7.61 (m, 3H), 7.42 (m, 1H), 7.26 (d, 1H), 6.75 (d, 1H), 6.40 (s, 1H), 2.62 (s, 2H), 2.45 (d, 3H), 1.90 (s, 3H), 1.53 (s, 6H); m/z 509.

### Example 66

### 3-(1-Cyano-1-methylethyl)-N-{4-methyl-3-[4-oxo-8-(1H-pyrazol-4-yl)-4H-quinazolin-3-yl]-phenyl}-benzamide

A microwave vial was charged with caesium carbonate (257 mg, 0.788 mmol), Pd(PPh₃)₄ (17 mg, 7.5% mmol), 4-(4,4,5,5-tetramethyl-[1,3,2]dioxaborolan-2-yl)-1H-pyrazole (46.5 mg, 0.24 mmol) and N-[3-(8-chloro-4-oxo-4H-quinazolin-3-yl)-4-methylphenyl]-3-(1-cyano-1-methylethyl)benzamide (Example 64; 90 mg, 0.197 mmol). The vial was fitted with a septum and purged with nitrogen. 1,4-Dioxane and water (4:1) (3 ml) was then added via syringe. The vial was irradiated in a microwave at 165° C. for 20 min. The mixture was then filtered through a pad of silica gel and washed with DCM. The filtrate was concentrated and purified first by column chromatography utilizing an ISCO system (0.5% triethyl amine, 5% methanol in DCM) and then by reverse phase chromatography utilizing a Gilson HPLC (0.1% TFA in Acetonitrile-water) to give 20 mg (20%) of a white solid. NMR (400 MHz): 10.25 (s, 1H), 8.12 (d, 3H), 7.90 (d, 1H), 7.82 (d, 1H), 7.80 (s, 1 h), 7.70 (d, 1H), 7.50 (m, 2H), 7.41 (d, 1H), 7.35 (m, 3H), 7.21 (d, 1H), 1.85 (s, 3H), 1.50 (s, 6H); m/z 489.

### Example 67

N-[3-(6-Bromo-4-oxo-4H-quinazolin-3-yl)-4-methylphenyl]-1-t-butyl-3-methyl-1H-pyrazole-5-carboxamide

A solution of 3-(5-amino-2-methylphenyl)-6-bromo-3H-quinazolin-4-one (Method 18; 100 mg, 0.46 mmol), 2-tert-butyl-5-methyl-2H-pyrazole-3-carbonyl chloride (93 mg, 0.46 mmol) and triethyl amine (92 mg, 0.92 mmol) in DCM (5 ml) was stirred at 25° C. for 1 hour. The reaction mixture was quenched with water (10 ml), and extracted with DCM (3×30 ml). The organics were dried over Na₂SO₄(s). The solvent was removed under reduced pressure and the resulting product was purified by column chromatography utilizing an ISCO system (hexane-EtOAc) to give 40 mg (17.6%) of a white solid. NMR (400 MHz): 10.09 (s, 1H), 8.70 (s, 1H), 8.60 (s, 1H), 8.35 (d, 1H), 8.21 (d, 1H), 8.15 (s, 1H), 8.05 (d, 1H), 7.70 (d, 1H), 6.90 (s, 1H), 2.80 (s, 3H), 2.35 (s, 3H), 1.90 (s, 9H); m/z 495.

### Example 68

### N-[3-(6-Morpholino-4-oxo-4H-quinazolin-3-yl)-4-methylphenyl]-1-t-butyl-3-methyl-1H-pyrazole-5-carboxamide

A microwave vial was charged with sodium tert-butoxide (8 mg, 0.06 mmol), Pd₂(dba)₃ (4 mg, 10% mmol), BINAP (5 mg, 20% mmol) and N-[3-(6-bromo-4-oxo-4H-quinazolin-3-yl)-4-methylphenyl]-1-t-butyl-3-methyl-1H-pyrazole-5-carboxamide (Example 67; 20 mg, 0.04 mmol). The vial was fitted with a septum, and flushed under nitrogen atmosphere. Morpholine (9 mg, 0.097 mmol) in 1,4-dioxane was then added dropwise via syringe. The vial was irradiated in a microwave at 175° C. for 30 min. The mixture was then filtered through a pad of silica gel and washed with DCM. The filtrate was concentrated and purified first by column chromatography utilizing an ISCO system (0.5% triethyl amine, 5% methanol in DCM) and then purified by reverse phase chromatography utilizing a Gilson HPLC (0.1% TFA in acetonitrile-water) to yield 15 mg (75%) of white solid. NMR (400 MHz): 10.70 (s, 1H), 8.10 (s, 1H), 7.90 (d, 1H), 7.80 (s, 1H), 7.65 (m, 2H), 7.50 (s, 1H), 7.40 (d, 1H), 6.55 (s, 1H), 3.80 (t, 4H), 3.21 (t, 4H), 2.45 (s, 3H), 2.05 (s, 3H), 1.62 (s, 9H); m/z 501.

### Example 69

### 3-(1-Cyano-1-methylethyl)-N-{4-methyl-3-[4-oxo-6-[(3-piperidin-1-ylpropyyl)amino]quinazolin-3(4H)-yl]phenyl}benzamide

Under an inert atmosphere, a 50 ml sealed tube was charged with a magnetic stirring bar, Pd₂(dba)₃ (45 mg, 0.049 mmol), BINAP (91 mg, 0.147 mmol), and toluene (5 ml). The complex was allowed to stir at 25° C. for 5 min before the addition of sodium tert-butoxide (0.191 g, 2.00 mmol), (3-piperidin-1-ylpropyl)amine-3-piperidin-1-ylpropan-1-amine (0.208 g, 1.47 mmol), and N-[3-(6-bromo-4-oxoquinazolin-3(4H)-yl)-4-methylphenyl]-3-(1-cyano-1-methylethyl)benzamide (Example 27; 0.250 g, 0.49 mmol). The reaction mixture was heated for 12 h at 100° C., cooled, quenched with water (100 ml), and extracted with EtOAc (2×100 ml). The combined organic extract was dried over MgSO₄, filtered, and concentrated in vacuo to yield the crude product which was purified on 40g SiO₂ using EtOAc-MeOH (4:1) as eluent to yield 0.220g (80%) as a white solid. NMR (400 MHz): 10.53 (s, 1H), 9.65 (m, 1H), 8.05 (s, 2H), 7.93 (d, 1H), 7.82-7.74 (m, 2H), 7.61 (d, 1H), 7.55 (d, 1H), 7.43 (d, 1H), 7.21 (dd, 1H), 7.16 (d, 1H), 3.39 (d, 2H), 3.20-3.12 (m, 5H), 2.87-2.85 (m, 3H), 2.05 (s, 3H), 2.04-1.98 (m, 4H), 1.80-1.62 (m, 3H), 1.74 (s, 6H); m/z 563.

### Examples 70-74

The following compounds were synthesized as described in Example 69 from N-[3-(6-bromo-4-oxoquinazolin-3(4H)-yl)-4-methylphenyl]-3-(1-cyano-1-methylethyl)benzamide (Example 27) and the appropriate amine.

| Ex | Compound | NMR | m/z | S.M |
|---|---|---|---|---|
| 70 | 3-(1-Cyano-1- | 10.50 (s, 1H), 8.06-8.04 (m, 2H), | 536 | (2S)-2- |
| | methylethyl)-N-{3- | 7.93 (d, 1H), 7.82-7.57 (m, 3H), | | (Methoxymethyl)- |
| | [6-[(2S)- | 7.64-7.57 (m, 2H), 7.43 (d, 1H), | | pyrrolidine |
| | 2-(methoxymethyl)- | 7.28 (d, 1H), 7.18 (m, 1H), | | |
| | pyrrolidin-1-yl]-4- | 4.58 (m, 2H), 3.49-3.42 (m, 2H), | | |
| | oxoquinazolin- | 3.28 (s, 3H), 3.29-3.10 (m, 1H), 2.05 (s, | | |
| | 3(4H)-yl]-4- | 3H), 2.04-1.98 (m, 2H), 1.74 (s, | | |
| | methylphenyl}benzamide | 6H), 1.73-1.62 (m, 2H) | | |
| 71 | N-{3-[6-(1,4′- | 10.55 (s, 1H), 9.93 (s, 1H), 8.14 (s, | 590 | 1,4′-Bipiperidine |
| | Bipiperidin-1′-yl)- | 1H), 8.05 (s, 1H), 7.93 (d, 1H), | | |
| | 4-oxoquinazolin- | 7.84-7.74 (m, 3H), 7.67 (s, 1H), | | |
| | 3(4H)-yl]-4- | 7.60 (t, 1H), 7.52 (s, 1H), 7.44 (d, | | |
| | methylphenyl}-3- | 1H), 4.03-3.99 (m, 1H), | | |
| | (1-cyano-1- | 3.39-3.25 (m, 4H), 2.99-2.85 (m, 4H), | | |
| | methylethyl)benzamide | 2.30-2.10 (m, 2H), 2.05 (s, 3H), | | |
| | | 2.04-1.98 (m, 2H), 1.85-1.70 (m, 6H), | | |
| | | 1.74 (s, 6H) | | |
| 72 | 3-(1-Cyano-1- | 10.48 (s, 1H), 8.03 (s, 1H), 7.97 (s, | 522 | 1- |
| | methylethyl)-N-{4- | 1H), 7.93 (d, 1H), 7.80-7.74 (m, | | (Tetrahydrofuran- |
| | methyl-3-[4-oxo-6- | 2H), 7.60 (t, 1H), 7.50 (d, 1H), | | 2-yl)methanamine |
| | [(tetrahydrofuran-2- | 7.42 (d, 1H), 7.28-7.24 (m, 2H), | | |
| | ylmethyl)amino]- | 7.16 (d, 1H), 4.05-4.00 (m, 1H), | | |
| | quinazolin-3(4H)- | 3.82-3.60 (m, 3H), 3.19-3.14 (m, | | |
| | yl]phenyl}benzamide | 2H), 2.04 (s, 3H), 2.02-1.72 (m, | | |
| | | 4H), 1.73 (s, 6H) | | |
| 73 | 3-(1-Cyano-1- | 10.50 (s, 1H), 9.88 (m, 1H), | 536 | N,N- |
| | methylethyl)-N-{3- | 8.06-8.03 (m, 2H), 7.93 (d, 1H), 7.83 (s, | | Dimethylpyrrolidin- |
| | [6-[3-(dimethyl- | 1H), 7.77 (t, 1H), 7.67 (d, 1H), | | 3-amine |
| | amino)pyrrolidin-1- | 7.60 (t, 1H), 7.43 (d, 1H), | | |
| | yl]-4- | 7.30-7.20 (m, 2H), 4.03-3.40 (m, 5H), | | |
| | oxoquinazolin-3(4H)- | 2.88 (t, 6H), 2.25-2.20 (m, 2H), | | |
| | yl]-4- | 2.05 (s, 3H), 1.73 (s, 6H) | | |
| | methylphenyl}benzamide | | | |
| 74 | 3-(1-Cyano-1- | 10.52 (s, 1H), 9.66 (m, 1H), | 550 | 1- |
| | methylethyl)-N-{3- | 8.17 (s, 1H), 8.03 (s, 1H), 7.93 (d, 1H), | | Isopropylpiperazine |
| | [6-(4-isopropyl- | 7.84 (d, 1H), 7.80-7.71 (m, 3H), | | |
| | piperazin-1-yl)-4- | 7.60-7.57 (m, 2H), 7.45 (d, 1H), | | |
| | oxoquinazolin- | 3.60-3.31 (m, 8H), 3.25-3.10 (m, | | |
| | 3(4H)-yl]-4- | 1H), 2.05 (s, 3H), 1.73 (s, 6H), | | |
| | methylphenyl}benzamide | 1.30 (d, 6H) | | |

### Example 75

### 3-(1-Cyano-1-methylethyl)-N-{3-[6-[3-(dimethylamino)propoxy]-4-oxoquinazolin-3(4H)-yl]-4-methylphenyl}benzamide

Under an inert atmosphere, a 10 ml sealed tube was charged with a magnetic stirring bar, Pd(OAc)₂ (8 mg, 0.012 mmol), BINAP (91 mg, 0.147 mmol), and toluene (1.5 ml). The catalyst was allowed to stir at 25° C. for 5 min before the addition of cesium carbonate (0.244 g, 0.75 mmol), N,N-dimethylaminopropanol (0.061 g, 0.600 mmol), and N-[3-(6-bromo-4-oxoquinazolin-3(4H)-yl)-4-methylphenyl]-3-(1-cyano-1-methylethyl)benzamide (Example 27; 0.150 g, 0.300 mmol). The reaction mixture was heated for 12 h at 40° C., cooled, quenched with water (50 ml), and extracted with EtOAc (2×50 ml). The combined organic extract was dried over MgSO₄, filtered, and concentrated in vacuo to yield the crude product which was purified on 40g SiO₂ using EtOAc-MeOH (4:1) as eluent to yield 0.039 g (25%) as a white solid. NMR (400 MHz): 10.52 (s, 1H), 10.06 (m, 1H), 8.23 (s, 1H), 8.05 (t, 1H), 7.94 (d, 1H), 7.86 (d, 1H), 7.81 (dd, 1H), 7.75 (d, 1H), 7.62-7.57 (m, 2H), 7.52 (dd, 1H), 7.44 (d, 1H), 4.21 (t, 2H), 3.27-3.20 (m, 2H), 2.80 (s, 3H), 2.78 (s, 3H), 2.20-2.15 (m, 2H), 2.05 (s, 3H), 1.74 (s, 6H); m/z 524.

### Examples 76-77

The following compounds were synthesized as described in Example 75 from N-[3-(6-bromo-4-oxoquinazolin-3(4H)-yl)-4-methylphenyl]-3-(1-cyano-1-methylethyl)benzamide (Example 27) and the appropriate alcohol.

| Ex | Compound | NMR | m/z | S.M |
|---|---|---|---|---|
| 76 | 3-(1-Cyano-1- | 10.53 (s, 1H), 9.98 (m, 1H), 8.22 (s, | 566 | 3-Morpholin- |
| | methylethyl)-N-{4- | 1H), 8.04 (s, 1H), 7.94 (d, 1H), | | 4-ylpropan-1- |
| | methyl-3-[6-(3- | 7.85 (d, 1H), 7.79-7.74 (m, 2H), | | ol |
| | morpholin-4- | 7.62-7.60 (m, 2H), 7.52 (dd, 1H), 7.44 (d, 1H), | | |
| | ylpropoxy)-4- | 4.03-3.93 (m, 2H), 3.70-3.65 (m, 2H), | | |
| | oxoquinazolin-3(4H)- | 3.48-3.40 (m, 6H), 3.14-3.04 (m, 2H), | | |
| | yl]phenyl}benzamide | 2.06 (s, 3H), 1.85-1.80 (m, 2H), | | |
| | | 1.74 (s, 6H) | | |
| 77 | 3-(1-Cyano-1- | 10.49 (s, 1H), 9.99 (m, 1H), 8.19 (s, | 580 | 3-(4-Methylpiperazin- |
| | methylethyl)-N-{4- | 1H), 8.04 (s, 1H), 7.99 (d, 1H), | | 1- |
| | methyl-3-[6-[3-(4- | 7.84 (d, 1H), 7.80-7.74 (m, 2H), | | yl)propan-1-ol |
| | methylpiperazin-1- | 7.62-7.58 (m, 2H), 7.52-7.50 (m, 1H), 7.42 (d, | | |
| | yl)propoxy]-4- | 1H), 4.01-3.95 (m, 2H), 3.14-3.04 (m, | | |
| | oxoquinazolin-3(4H)- | 8H), 3.14 (s, 3H), 2.05 (s, 3H), | | |
| | yl]phenyl}benzamide | 1.84-1.80 (m, 2H), 1.77 (s, 6H) | | |

### Example 78

### N-Cyclopropyl-3-(2-methyl-5-{[3-(trifluoromethyl)benzoyl]amino}phenyl)-4-oxo-3 4-dihydroquinazoline-8-carboxamide

A mixture of 3-[2-Methyl-5-(3-trifluoromethyl-benzoylamino)-phenyl]-4-oxo-3,4-dihydro-quinazoline-8-carboxylic acid (Example 2; 47 mg, 0.10 mmol), cyclopropylamine (0.1 ml), HATU (45 mg, 0.12 mmol) and DIEA (64.5 mg, 0.5 mmol) in 2 ml of anhydrous DMF was stirred at 25° C. for 2 h. Water (5 ml) was then added to the mixture and concentrated under reduced pressure until solid started to precipitate from water. The solid was collected by filtration and purified by column chromatography (silica gel) using hexane-EtOAc to yield 35 mg of white solid (69.1%). NMR (400 MHz): 10.73 (s, 1H), 9.92 (s, 1H), 8.57 (s, 1H), 8.49 (d, 2H), 8.40 (d, 1H), 8.32 (m, 2H), 8.05 (d, 1H), 7.96 (s, 1H), 7.85 (m, 2H), 7.75 (t, 1H), 7.50 (d, 1H), 3.00 (m, 1H), 2.15 (s, 3H), 0.85 (m, 2H), 0.60 (m, 2H); m/z 507.

### Example 79

The following compound was prepared by the procedure of Example 78, using 3-[2-methyl-5-(3-trifluoromethyl-benzoylamino)-phenyl]-4-oxo-3,4-dihydro-quinazoline-8-carboxylic acid (Example 2) and the appropriate amine.

| Ex | Compound | NMR | m/z | S.M |
|---|---|---|---|---|
| 79 | N-[2-(Dimethylamino)ethyl]- | 10.90 (s, 1H), 10.30 (s, br, 1H), | 538 | N,N-Dimethylethane- |
| | 3-(2-methyl-5-{[3- | 10.10 (t, 1H), 8.60-8.32 (m, 5H), | | 1,2-diamine |
| | (trifluoromethyl)benzoyl]- | 8.05-7.75 (m, 5H), 7.55 (d, 1H), | | |
| | amino}phenyl)-4-oxo- | 3.90 (m, 2H), 3.40 (m, 2H), | | |
| | 3,4-dihydroquinazoline- | 3.95 (s, 6H), 2.19 (s, 3H) | | |
| | 8-carboxamide | | | |

### Example 80

### 3-(5-{[3-(1-Cyano-1-methylethyl)benzoyl]amino}-2-methylphenyl)-N-methyl-4-oxo-3,4-dihydroquinazoline-8-carboxamide

A mixture of 3-(5-{[3-(1-cyano-1-methylethyl)benzoyl]amino}-2-methylphenyl)-4-oxo-3,4-dihydroquinazoline-8-carboxylic acid (Example 3; 100 mg, 0.21 mmol), methylamine hydrochloride (134 mg, 2 mmol), HATU (98 mg, 0.26 mmol) and DIEA (277 mg, 2.1 mmol) in 2 ml of anhydrous DMF was stirred at 25° C. for 2 h. The product was purified by using an ISCO system (hexane-EtOAc) to give 70 mg of white solid (69.6%). NMR (400 MHz): 10.36 (s, 1H), 9.60 (t, 1H), 8.35 (s, 1H), 8.30 (d, 1H), 8.20 (d, 1H), 7.89 (s, 1H), 7.78 (d, 1H), 7.75 (s, 1H), 7.65 (d, 1H), 7.55 (m, 2H), 7.45 (t, 1H), 7.30 (d, 1H), 2.79 (s, 3H), 1.95 (s, 3H), 1.60 (s, 6H); m/z 479.

### Examples 81-90

The following compounds were prepared by the procedure of Example 80, using 3-(5-{[3-(1-cyano-1-methylethyl)benzoyl]amino}-2-methylphenyl)-4-oxo-3,4-dihydroquinazoline-8-carboxylic acid (Example 3), 3-[2-methyl-5-(3-trifluoromethyl-benzoylamino)-phenyl]-4-oxo-3,4-dihydro-quinazoline-8-carboxylic acid (Example 2) or 6-bromo-3-{5-[3-(cyano-dimethyl-methyl)-benzoylamino]-2-methyl-phenyl}-4-oxo-3,4-dihydro-quinazoline-8-carboxylic acid (Example 4) and the appropriate starting amine.

| Ex | Compound | NMR | m/z | S.M |
|---|---|---|---|---|
| 81 | 3-(5-{[3-(1-Cyano-1- | 10.60 (s, 1H), 10.25 (t, 1H), | 525 | (2- |
| | methylethyl)benzoyl]- | 8.65 (m, 2H), 8.45 (d, 1H), | | Methoxyethyl)- |
| | amino}-2-methylphenyl)- | 8.10 (s, 1H), 7.98 (m, 2H), | | amine |
| | N-(2-methoxyethyl)-4- | 7.88-7.75 (m, 3H), 7.65 (t, | | |
| | oxo-3,4- | 1H), 7.52 (d, 1H), 3.65 (m, | | |
| | dihydroquinazoline-8- | 4H), 3.35 (s, 3H), 2.20 (s, 3H), | | |
| | carboxamide | 1.80 (s, 6H) | | |
| 82 | 3-(5-{[3-(1-Cyano-1- | 10.71 (s, 1H), 10.05 (m, 2H), | 537 | N,N- |
| | methylethyl)benzoyl]amino}- | 8.60 (s, 1H), 8.57 (d, 1H), | | Dimethylethane- |
| | 2-methylphenyl)- | 8.50 (d, 1H), 8.15 (s, 1H), 8.06 (m, | | 1,2-diamine |
| | N-[2-(dimethylamino)ethyl]- | 2H), 7.85 (m, 3H), 7.70 (t, | | |
| | 4-oxo-3,4- | 1H), 7.53 (d, 1H), 3.90 (m, | | |
| | dihydroquinazoline-8- | 2H), 3.40 (m, 2H), 2.95 (s, | | |
| | carboxamide | 6H), 2.18 (s, 3H), 1.82 (s, 6H) | | |
| 83 | 3-(5-{[3-(1-Cyano-1- | 10.56 (s, 1H), 9.95 (d, 1H), | 507 | Cyclopropyl- |
| | methylethyl)benzoyl]amino}- | 8.55 (s, 1H), 8.48 (d, 1H), | | amine |
| | 2-methylphenyl)- | 8.40 (d, 1H), 8.10 (s, 1H), 7.98 (m, | | |
| | N-cyclopropyl-4-oxo- | 2H), 7.82 (m, 2H), 7.77 (t, | | |
| | 3,4-dihydroquinazoline- | 1H), 7.66 (t, 1H), 7.50 (d, 1H), | | |
| | 8-carboxamide | 3.00 (m, 1H), 2.16 (s, 3H), | | |
| | | 1.80 (s, 6H), 0.85 (m, 2H), | | |
| | | 0.65 (m, 2H) | | |
| 84 | 3-(5-{[3-(1-Cyano-1- | 12.60 (s, 1H), 10.60 (s, 1H), | 532 | 1H-Pyrazol-3- |
| | methylethyl)benzoyl]amino}- | 8.72 (m, 2H), 8.50 (d, 1H), | | amine |
| | 2-methylphenyl)- | 8.10 (s, 1H), 8.00 (m, 2H), | | |
| | 4-oxo-N-1H-pyrazol-3- | 7.90-7.76 (m, 4H), 7.70 (t, | | |
| | yl-3,4- | 1H), 7.55 (d, 1H), 6.80 (s, 1H), | | |
| | dihydroquinazoline-8- | 2.18 (s, 3H), 0.80 (s, 6H) | | |
| | carboxamide | | | |
| 85 | 3-(5-{[3-(1-Cyano-1- | 10.60 (s, 1H), 9.85 (t, 1H), | 560 | 2-(1H-Imidazol- |
| | methylethyl)benzoyl]amino}- | 9.02 (s, 1H), 8.50 (s, 1H), | | 4-yl)ethanamine |
| | 2-methylphenyl)- | 8.40 (m, 2H), 8.10 (s, 1H), 8.00 (m, | | |
| | N-[2-(1H-imidazol-4- | 2H), 7.75 (m, 3H), 7.63 (m, | | |
| | yl)ethyl]-4-oxo-3,4- | 2H), 7.50 (d, 1H), 3.73 (m, | | |
| | dihydroquinazoline-8- | 2H), 3.02 (t, 2H), 2.15 (s, 3H), | | |
| | carboxamide | 1.80 (s, 6H) | | |
| 86 | 3-(5-{[3-(1-Cyano-1- | 14.00 (s, 1H), 10.45 (s, 1H), | 550 | 1,3,4-Thiadiazol- |
| | methylethyl)benzoyl]amino}- | 9.23 (s, 1H), 8.70 (s, 1H), | | 2-amine |
| | 2-methylphenyl)- | 8.55 (d, 1H), 8.45 (d, 1H), 8.00 (s, | | |
| | 4-oxo-N-1,3,4-thiadiazol- | 1H), 7.85 (m, 2H), 7.70 (m, | | |
| | 2-yl-3,4- | 3H), 7.50 (t, 1H), 7.40 (d, 1H), | | |
| | dihydroquinazoline-8- | 2.10 (s, 3H), 1.70 (s, 6H) | | |
| | carboxamide | | | |
| 87 | 3-(5-{[3-(1-Cyano-1- | 14.00 (s, 1H), 10.60 (s, 1H), | 564 | 5-Methyl-1,3,4- |
| | methylethyl)benzoyl]amino}- | 8.80 (s, 1H), 8.65 (d, 1H), | | thiadiazol-2- |
| | 2-methylphenyl)- | 8.55 (d, 1H), 8.10 (s, 1H), 7.96 (m, | | amine |
| | N-(5-methyl-1,3,4- | 2H), 7.85 (m, 3H), 7.60 (t, | | |
| | thiadiazol-2-yl)-4-oxo- | 1H), 7.45 (s, 1H), 2.75 (s, 3H), | | |
| | 3,4-dihydroquinazoline- | 2.15 (s, 3H), 1.80 (s, 6H) | | |
| | 8-carboxamide | | | |
| 88 | 3-(2-Methyl-5-{[3- | 10.75 (s, 1H), 9.21 (s, 2H), | 467 | Ammonium |
| | (trifluoromethyl)benzoyl]- | 8.85 (s, 1H), 8.55 (m, 2H), | | chloride |
| | amino}phenyl)-4-oxo- | 8.30 (m, 2H), 8.01 (m, 2H), | | |
| | 3,4-dihydroquinazoline- | 7.70 (m, 3H), 7.55 (d, 1H), | | |
| | 8-carboxamide | 2.20 (s, 3H) | | |
| 89 | 3-(5-{[3-(1-Cyano-1- | 10.75 (s, 1H), 9.21 (s, 2H), | 468 | Ammonium |
| | methylethyl)benzoyl]amino}- | 8.85 (s, 1H), 8.55 (m, 2H), | | chloride |
| | 2-methylphenyl)- | 8.30 (m, 2H), 8.01 (m, 2H), | | |
| | 4-oxo-3,4- | 7.70 (m, 3H), 7.55 (d, 1H), | | |
| | dihydroquinazoline-8- | 3.95 (s, 6H), 2.19 (s, 3H) | | |
| | carboxamide | | | |
| 90 | 6-Bromo-3-(5-{[3-(1- | 10.25 (s, 1H), 9.50 (s, 1H), | 585 | Cyclopropyl- |
| | cyano-1-methylethyl)- | 8.30 (s, 1H), 8.20 (m, 2H), | | amine |
| | benzoyl]amino}-2- | 7.80 (s, 1H), 7.70 (m, 2H), | | |
| | methylphenyl)-N- | 7.55 (m, 2H), 7.40 (t, 1H), | | |
| | cyclopropyl-4-oxo-3,4- | 7.20 (d, 1H), 2.70 (m, 1H), 1.85 (s, | | |
| | dihydroquinazoline-8- | 3H), 1.50 (s, 6H), 0.50 (m, | | |
| | carboxamide | 2H), 0.30 (m, 2H) | | |

### Example 91

### 3-(1-Cyano-1-methylethyl)-N-{3-[8-[(cyclopropylcarbonyl)amino]-4-oxoquinazolin-3 (4H)-yl]-4-methylphenyl}benzamide

A mixture of N-[3-(8-amino-4-oxo-4H-quinazolin-3-yl)-4-methyl-phenyl]-3-(cyano-dimethyl-methyl)-benzamide (Example 106; 100 mg, 0.23 mmol), cyclopropanecarbonyl chloride (0.2 ml) and triethylamine (46 mg, 0.46 mmol) in DCM (5 ml) was stirred at 25° C. for 12 h. The product was purified by an ISCO system (hexane-EtOAc) to give 45 mg of white solid (38.7%). NMR (400 MHz): 10.60 (s, 1H), 10.20 (s, 1H), 8.70 (d, 1H), 8.50 (s, 1H), 8.15 (s, 1H), 8.00 (m, 4H), 7.85 (d, 1H), 7.75 (m, 2H), 7.62 (d, 1H), 2.35 (m, 1H), 2.18 (s, 3H), 1.80 (s, 6H), 0.95 (m, 4H); m/z 506.

### Examples 92-93

The following compounds were prepared by the procedure of Example 91, using N-[3-(8-amino-4-oxo-4H-quinazolin-3-yl)-4-methyl-phenyl]-3-(cyano-dimethyl-methyl)-benzamide (Example 106) and the appropriate starting material.

| Ex | Compound | NMR | m/z | S.M |
|---|---|---|---|---|
| 92 | 3-(1-Cyano-1- | 10.50 (s, 1H), 9.35 (s, 1H), | 516 | Methane sulfonyl |
| | methylethyl)-N-{4- | 8.50 (s, 1H), 8.10 (s, 1H), | | chloride |
| | methyl-3-[8- | 8.05 (m, 2H), 7.90 (m, 2H), 7.89 (d, | | |
| | [(methylsulfonyl)amino]- | 1H), 7.80 (m, 1H), 7.60 (t, | | |
| | 4-oxoquinazolin-3(4H)- | 2H), 7.50 (d, 1H), 3.30 (s, 3H), | | |
| | yl]phenyl}benzamide | 2.15 (s, 3H), 1.80 (s, 6H) | | |
| 93 | N-[3-(5-{[3-(1-Cyano-1- | 11.25 (s, 1H), 10.65 (s, 1H), | 550 | 1,2,3- |
| | methylethyl)benzoyl]amino}- | 10.00 (s, 1H), 9.00 (d, 1H), | | Thiadiazole-4- |
| | 2-methylphenyl)-4- | 8.60 (s, 1H), 8.12 (s, 1H), | | carbonyl chloride |
| | oxo-3,4- | 8.05 (m, 2H), 7.95 (m, 2H), | | |
| | dihydroquinazolin-8-yl]- | 7.80 (m, 2H), 7.65 (t, 1H), 7.50 (d, | | |
| | 1,2,3-thiadiazole-4- | 1H), 2.18 (s, 3H), 1.80 (s, 6H) | | |
| | carboxamide | | | |

### Example 94

### 3-(Cyano-dimethyl-methyl)-N-{4-methyl-3-[8-(3-methyl-ureido)-4-oxo-4H-quinazolin-3-yl]-phenyl}benzamide

A suspension of 3-(5-{[3-(1-cyano-1-methylethyl)benzoyl]amino}-2-methylphenyl)-4-oxo-3,4-dihydroquinazoline-8-carboxylic acid (Example 3; 150 mg, 0.32 mmol), diphenyl phosphoryl azide (177 mg, 0.64 mmol) and DIEA (83 mg, 0.64 mmol) in toluene (10 ml) was stirred at reflux for 5 h. Methylamine (2 M in THF, 5 ml) was then added to the suspension and the reaction mixture was again stirred at reflux for 1 h. The clear solution was treated with a second portion of methylamine (2 M in THF, 5 ml) and the resulting mixture was stirred at 100° C. for 2 days. The product was purified by an ISCO system (hexane-EtOAc) to yield 30 mg of white solid (19%). NMR (400 MHz): 10.30 (s, 1H), 8.80 (s, 1H), 8.47 (d, 1H), 8.15 (s, 1H), 7.86 (s, 1H), 7.75 (d, 1H), 7.65 (m, 2H), 7.59 (d, 1H), 7.55 (d, 1H), 7.45 (m, 1H), 7.30 (m, 2H), 7.06 (m, 1H), 2.50 (d, 3H), 1.90 (s, 3H), 1.60 (s, 6H); m/z 495.

### Example 95

### 3-(Cyano-dimethyl-methyl)-N-{3-[8-(2-methoxy-ethylamino)-4-oxo-4H-quinazolin-3-yl]-4-methyl-phenyl}benzamide

A microwave vial was charged with cesium carbonate (161 mg, 0.49 mmol), Pd₂(dba)₃ (30 mg, 10% mmol), tri-tert-butylphosphine (0.15 ml) and N-[3-(8-chloro-4-oxo-4H-quinazolin-3-yl)-4-methylphenyl]-3-(1-cyano-1-methylethyl)benzamide (Example 64; 150 mg, 0.329 mmol). The vial was fitted with a septum and purged with nitrogen. 2-Methoxyethylamine (49 mg, 0.658 mmol) in 1,4-dioxane (3 ml) was then added via syringe. The vial was irradiated in a microwave at 165° C. for 20 min. The mixture was filtered through a pad of silica gel and washed with DCM. The filtrate was concentrated and then purified by column chromatography utilizing an ISCO system (hexane-EtOAc) to give 90 mg (55.3%) of a white solid. NMR (400 MHz): 10.30 (s, 1H), 8.10 (s, 1H), 7.90 (s, 1H), 7.75 (d, 1H), 7.70 (m, 3H), 7.45 (m, 1H), 7.30-7.20 (m, 3H), 6.80 (d, 1H), 5.92 (t, 1H), 3.45 (m, 2H), 3.25 (m, 5H), 1.90 (s, 3H), 1.60 (s, 6H); m/z 496.

### Example 96

The following compounds were prepared by the procedure of Example 95, using 6-bromo-3-(5-{[3-(1-cyano-1-methylethyl)benzoyl]amino}-2-methylphenyl)-N-cyclopropyl-4-oxo-3,4-dihydroquinazoline-8-carboxamide (Example 90) and the appropriate starting material.

| Ex | Compound | NMR | m/z | S.M |
|---|---|---|---|---|
| 96 | 3-(5-{[3-(1-Cyano-1- | 10.28 (s, 1H), 9.65 (s, 1H), 9.41 (s, | 604 | N-Methyl |
| | methylethyl)benzoyl]amino}- | br, 1H), 8.10 (s, 1H), 7.90 (s, 1H), | | piperizine |
| | 2-methylphenyl)-N- | 7.80 (s, 1H), 7.65 (m, 2H), | | |
| | cyclopropyl-6-(4- | 7.60 (m, 3H), 7.36 (t, 1H), 7.20 (d, 1H), | | |
| | methylpiperazin-1-yl)-4-oxo- | 3.10 (m, 8H), 2.62 (m, 4H), | | |
| | 3,4-dihydroquinazoline-8- | 1.82 (s, 3H), 1.40 (s, 6H), 0.50 (m, 2H), | | |
| | carboxamide | 0.30 (m, 2H) | | |

### Example 97

### 3-(Cyano-dimethyl-methyl)-N-[4-methyl-3-(4-oxo-7-pyridin-3-yl-4H-quinazolin-3-yl)-phenyl]-benzamide

A microwave vial was charged with caesium carbonate (389 mg, 1.196 mmol), Pd(PPh₃)₄ (26 mg, 7.5% mmol), N-[3-(7-bromo-4-oxo-4H-quinazolin-3-yl)-4-methylphenyl]-3-(1-cyano-1-methylethyl)benzamide (Example 51; 150 mg, 0.299 mmol) and 3-pyridine boronic acid (36.7 mg, 0.299 mmol). The vial was fitted with a septum and purged with nitrogen. 1,4-Dioxane-water (4:1), (3 ml) was added via a syringe. The vial was irradiated in a microwave at 165° C. for 20 min. The mixture was then filtered through a pad of silica gel and washed with DCM. The filtrate was concentrated and the resulting solid was purified by a Gilson HPLC (5-95% acetonitrile-water-0.1% TFA) to give 83 mg (55.6%). NMR (400 MHz): 10.35 (s, 1H), 9.05 (s, 1H), 8.65 (s, 1H), 8.40 (d, 1H), 8.25 (s, 1H), 8.20 (d, 1H), 8.05 (s, 1H), 7.90 (m, 2H), 7.80 (d, 1H), 7.72 (s, 1H), 7.50 (m, 3H), 7.42 (t, 1H), 7.25 (d, 1H), 1.95 (s, 3H), 1.60 (s, 6H); m/z 500.

### Example 98

### 3-(Cyano-dimethyl-methyl)-N-{3-[8-(2-diethylamino-ethoxy)-4-oxo-4H-quinazolin-3-yl]-4-methyl-phenyl}benzamide

A suspension of 3-(1-cyano-1-methylethyl)-N-[3-(8-hydroxy-4-oxo-4H-quinazolin-3-yl)-4-methylphenyl]benzamide (Example 63; 96 mg, 0.219 mmol), 2-diethylamino ethyl chloride hydrochloride (49 mg, 0.285 mmol), potassium carbonate (302 mg, 2.19 mmol) and sodium iodide (3 mg, 0.0219 mmol) in acetone (10 ml) was refluxed for 12 h. The solid was filtered, washed with acetone, and discarded. The filtrate was concentrated and the resulting residue was purified by a Gilson HPLC (5-90% acetonitrile-water-0.1% TFA) to give 65 mg of white solid (55.3%). NMR (400 MHz): 10.68 (s, 1H), 10.40 (s, br, 1H), 8.40 (s, 1H), 8.12 (s, 1H), 8.00 (d, 1H), 7.90 (m, 3H), 7.80 (d, 1H), 7.60 (m, 3H), 7.50 (d, 1H), 4.60 (t, 2H), 3.50 (m, 6H), 2.15 (s, 3H), 1.80 (s, 6H), 1.35 (t, 6H); m/z 539.

### Example 99

### 3-(1-Cyano-1-methylethyl)-N-{3-[6-[3-(dimethylamino)prop-1-yn-1-yl]-4-oxoquinazolin-3(4H)-yl]-4-methylphenyl}benzamide

N-[3-(6-Bromo-4-oxo-4H-quinazolin-3-yl)-4-methylphenyl]-3-(1-cyano-1-methylethyl)benzamide (Example 27; 0.250g, 0.500 mmol) was added to acetonitrile (4.00 ml). Triethylamine (0.350 ml, 2.50 mmol) was added followed by N,N-dimethylprop-2-yn-1-amine (0.103 g, 1.25 mmol). With stirring Pd(PPh₃)₄ (57 mg, 0.05 mmol) and CuI (10 mg, 0.050 mmol) were added and the reaction was warmed to 60° C. for 4 h. The reaction was then diluted with EtOAc (50 ml) and filtered through a pad of SiO₂, and concentrated in vacuo. The crude product was purified on 40g SiO₂ using EtOAc-MeOH 10:1 as eluent giving 0.203 g (81%). NMR (400 MHz): 11.02 (brs, 1H), 10.60 (s, 1H), 8.41 (s, 1H), 8.34 (d, 1H), 8.06 (s, 1H), 7.98 (dd, 1H), 7.92 (d, 1H), 7.89 (s, 1H), 7.82 (d, 1H), 7.75 (d, 1H), 7.59 (t, 1H), 7.44 (d, 1H), 4.36 (d, 2H), 2.88 (s, 3H), 2.87 (s, 3H) 2.07 (s, 3H), 1.74 (s, 6H); m/z 504.

### Examples 100-102

The following compounds were prepared by the procedure of Example 99, N-[3-(6-bromo-4-oxo-4H-quinazolin-3-yl)-4-methylphenyl]-3-(1-cyano-1-methylethyl)benzamide (Example 27) and the appropriate starting alkyne.

| Ex | Compound | NMR | m/z | S.M |
|---|---|---|---|---|
| 100 | 3-(1-Cyano-1- | 10.59 (s, 1H), 9.35-9.28 (m, | 490 | N-Methylprop-2- |
| | methylethyl)-N-{4- | 1H), 8.41 (s, 1H), 8.29 (s, 1H), | | yn-1-amine |
| | methyl-3-[6-[3- | 8.06 (s, 1H), 7.98-7.95 (m, | | |
| | (methylamino)prop-1- | 2H), 7.90 (s, 1H), | | |
| | yn-1-yl]-4-oxo | 7.83-7.73 (m, 2H), 7.59 (t, 1H), 7.45 (d, | | |
| | quinazolin-3(4H)- | 1H), 4.19 (t, 2H), 3.56 (s, 1H), | | |
| | yl]phenyl}benzamide | 2.67 (s, 3H), 2.07 (s, 3H), | | |
| | | 1.73 (s, 6H) | | |
| 101 | N-{3-[6-(3-Aminoprop- | 10.59 (s, 1H), 8.60-8.48 (m, | 476 | Prop-2-yn-1- |
| | 1-yn-1-yl)-4-oxo | 3H), 8.25 (s, 1H), 8.06 (s, 1H), | | amine |
| | quinazolin-3(4H)-yl]-4- | 7.94-7.89 (m, 2H), | | |
| | methylphenyl}-3-(1- | 7.83-7.76 (m, 2H), 7.59 (t, 1H), 7.46 (d, | | |
| | cyano-1-methylethyl) | 1H), 4.05 (m, 2H), 2.07 (s, | | |
| | benzamide | 3H), 1.74 (s, 6H) | | |
| 102 | N-{3-[6-[3- | 10.52 (s, 1H), 8.43 (t, 1H), | 518 | N-Prop-2-yn-1- |
| | (Acetylamino)prop-1- | 8.36 (s, 1H), 8.16 (d, 1H), | | ylacetamide |
| | yn-1-yl]-4-oxo | 8.04 (s, 1H), 7.92-7.74 (m, 3H), | | |
| | quinazolin-3(4H)-yl]-4- | 7.64-7.52 (m, 3H), 7.44 (d, | | |
| | methylphenyl}-3-(1- | 1H), 4.14 (d, 2H), 2.07 (s, 3H), | | |
| | cyano-1-methylethyl) | 1.85 (s, 3H), 1.74 (s, 6H) | | |
| | benzamide | | | |

### Example 103

### 3-(1-Cyano-1-methylethyl)-N-{4-methyl-3-[6-[3-(methylamino)propyl]-4-oxoquinazolin-3(4H)-yl]phenyl}benzamide

3-(1-Cyano-1-methylethyl)-N-{4-methyl-3-[6-[3-(methylamino)prop-1-yn-1-yl]-4-oxoquinazolin-3(4H)-yl]phenyl}benzamide (Example 100; 0.05 g, 0.102 mmol) was dissolved in MeOH (5 ml). Palladium on carbon (10 wt %) was then added and the reaction was placed under 1 atmosphere of hydrogen and stirred for 8 h at 25° C. The reaction mixture was filtered through celite and concentrated in vacuo to yield the crude product which was purified on 40 g SiO₂ using EtOAc-MeOH 4:1 as eluent to yield 0.040g (79%) of a white solid. NMR (400 MHz): 10.53 (s, 1H), 8.40-8.38 (m, 1H), 8.30 (s, 1H), 8.08 (s, 1H), 8.03 (s, 1H), 7.93 (d, 1H), 7.86 (s, 1H), 7.80-7.76 (m, 3H), 7.59 (t, 1H), 7.45 (d, 1H), 3.00 (s, 3H), 2.98-2.96 (m, 2H), 2.07 (s, 3H), 1.85-1.82 (m, 2H), 1.74 (s, 6H), 1.65-1.62 (m, 2H); m/z 494.

### Examples 104-105

The following compounds were prepared by the procedure of Example 103 utilizing the appropriate starting material.

| Ex | Compound | NMR | m/z | S.M |
|---|---|---|---|---|
| 104 | 3-(1-Cyano-1- | 10.49 (s, 1H), 8.41 (t, 1H), 8.29 (s, | 508 | Example 99 |
| | methylethyl)-N-{3- | 1H), 8.08 (s, 1H), 8.02 (s, 1H), | | |
| | [6-[3-(dimethyl | 7.95 (d, 1H), 7.88 (d, 1H), 7.80 (d, | | |
| | amino)propyl]-4- | 2H), 7.75 (s, 1H), 7.59 (t, 1H), | | |
| | oxo quinazolin- | 7.45 (d, 1H), 3.00 (s, 3H), 2.99 (s, | | |
| | 3(4H)-yl]-4- | 3H), 2.98-2.96 (m, 2H), 2.06 (s, | | |
| | methylphenyl} | 3H), 1.85-1.80 (m, 2H), 1.77 (s, | | |
| | benzamide | 6H), 1.70-1.66 (m, 2H) | | |
| 105 | N-{3-[6-[3- | 10.51 (s, 1H), 10.46 (s, 1H), | 522 | Example 102 |
| | (Acetylamino)propyl]- | 8.40 (t, 1H), 8.25 (s, 1H), 8.09 (s, 1H), | | |
| | 4- | 8.05 (s, 1H), 7.95 (t, 1H), 7.90 (d, | | |
| | oxoquinazolin- | 2H), 7.81 (d, 1H), 7.77 (s, 1H), | | |
| | 3(4H)-yl]-4- | 7.60 (t, 1H), 7.45 (d, 1H), | | |
| | methylphenyl}-3- | 3.10-3.08 (m, 2H), 2.07 (s, 3H), 1.88 (s, | | |
| | (1-cyano-1- | 3H), 1.86-1.83 (m, 2H), 1.77 (s, | | |
| | methylethyl) | 6H), 1.71-1.64 (m, 2H) | | |
| | benzamide | | | |

### Example 106

### N-[3-(8-Amino-4-oxo-4H-quinazolin-3-yl)-4-methyl-phenyl]-3-(cyano-dimethyl-methyl)-benzamide

A suspension of 3-(5-{[3-(1-cyano-1-methylethyl)benzoyl]amino}-2-methylphenyl)-4-oxo-3,4-dihydroquinazoline-8-carboxylic acid (Example 3; 466 mg, 1 mmol), diphenyl phosphoryl azide (550 mg, 2 mmol) and DIEA (258 mg, 2 mmol) in tert-butanol was stirred to reflux for 12 h. The clear solution was cooled to 25° C. and concentrated under reduced pressure. The resulting residue was purified with an ISCO system (hexane-EtOAc) to yield 293 mg. The solid was then treated with 4 M HCl in dioxane (3 ml) for 2 h at 25° C. and concentrated under reduced pressure. The resulting residue was purified by a Gilson HPLC (5-95% acetonitrile-water-0.1% TFA) to yield 153 mg of white solid (35%). NMR (400 MHz): 10.40 (s, 1H), 8.10 (s, 1H), 7.95 (s, 1H), 7.85 (d, 1H), 7.70 (m, 3H), 7.49 (t, 1H), 7.30 (d, 1H), 7.20 (m, 2H), 6.98 (d, 1H), 1.95 (s, 3H), 1.60 (s, 6H); m/z 438.

### Examples 107-108

The following compounds were synthesized as described in Example 27 from 3-(5-amino-2-methylphenyl)-6-(4-methyl-1,4-diazepan-1-yl)quinazolin-4(3H)-one (Method 40) or 3-(5-amino-2-methylphenyl)-6-(4-methylpiperazin-1-yl)quinazolin-4(3H)-one (Method 41) and 3-(1-cyano-1-methylethyl)benzoic acid (Method 11).

| Ex | Compound | NMR | m/z |
|---|---|---|---|
| 107 | 3-(1-Cyano-1- | 10.28 (s, 1H), 7.92 (m, 1H), 7.84 (s, 1H), | 535 |
| | methylethyl)-N-{4-methyl- | 7.82 (d, 1H), 7.62 (m, 3H), 7.46 (m, 2H), 7.28 (d, | |
| | 3-[6-(4-methyl-1,4- | 1H), 7.24 (d, 1H), 7.12 (d, 1H), 3.48 (m, 2H), | |
| | diazepan-1-yl)-4- | 3.42 (m, 2H), 3.18 (m, 2H), 2.52 (m, 2H), | |
| | oxoquinazolin-3(4H)- | 2.14 (s, 3H), 1.92 (s, 3H), 1.80 (m, 2H), 1.62 (s, 6H) | |
| | yl]phenyl}benzamide | | |
| 108 | 3-(1-Cyano-1- | 8.62 (s, 1H), 7.92 (m, 1H), 7.82 (m, 2H), | 521 |
| | methylethyl)-N-{4-methyl- | 7.68 (m, 2H), 7.62 (m, 3H), 7.46 (m, 2H), 7.24 (d, | |
| | 3-[6-(4-methylpiperazin-1- | 1H), 3.38 (m, 4H), 2.60 (m, 4H), 2.38 (s, 3H), | |
| | yl)-4-oxoquinazolin-3(4H)- | 1.94 (s, 3H), 1.84 (s, 3H), 1.74 (s, 6H) | |
| | yl]phenyl}benzamide | | |

### Preparation of Starting Materials

### Method 1

### N-(4-Methyl-3-nitrophenyl)-3-trifluoromethylbenzamide

A solution of 4-methyl-3-nitro-phenylamine (3.64 g, 24 mmol) and 3-trifluoromethyl-benzoyl chloride (5 g, 24 mmol) in DCM (100 ml) was treated with triethylamine (4.85 g, 48 mmol). The mixture was stirred at 25° C. for 20 min. The reaction was then quenched with water (50 ml) and stirred for 15 min. The solid was collected by vacuum filtration and washed with hexane. A second crop of solid was collected from the filtrate to give a total yield of 7.78 g (100%) of white-light yellow solid. NMR (400 MHz): 7.35 (m, 1H), 7.66 (m, 1H), 7.87 (m, 2H), 8.15 (m, 2H), 8.40 (s, 1H), 10.62 (s, 1H); m/z 324.

### Method 2

### N-(3-Amino-4-methylphenyl)-3-trifluoromethylbenzamide

A suspension of N-(4-methyl-3-nitrophenyl)-3-trifluoromethylbenzamide (Method 1; 324 mg, 1 mmol) and tin (II) chloride (1.33 g, 7 mmol) in DMF (2 ml) was stirred at 25° C. for 12 hours. The mixture was treated with 25% NaOH (10 ml) and extracted with chloroform (3×50 ml). The organic phases were combined and dried over anhydrous sodium sulfate and concentrated. The resulting product was purified by column chromatography utilizing an ISCO system (hexane-EtOAc) to yield 270 mg (92%) as a white solid. NMR (400 MHz): 10.00 (s, 1H), 8.05 (m, 2H), 7.80 (m, 1H), 7.60 (m, 1H), 6.92 (s, 1H), 6.70m (m, 2H), 4.70 (s, 2H), 1.87 (s, 3H); m/z 294.

### Method 3

### 2-Amino-4-bromobenzoic acid

A solution of 2-amino-4-bromobenzoic acid ethyl ester (6 g, 24.5 mmol) in 84 ml of ethanol was treated with sodium hydroxide (1.97 g in 17 ml water). The reaction mixture was stirred at 25° C. for 12 hours. The ethanol was removed by distillation and the resulting suspension was diluted with water (200 ml) and acidified with 10% HCl to pH=1-3. The white solid was collected by filtration, washed with water and dried via high vacuum (5.2 g, 98.3%). NMR (400 MHz): 7.50 (d, 1H), 6.90 (s, 1H), 6.55 (d, 1H); m/z 216.

### Method 4

### 3-Cyanomethyl-benzoic acid methyl ester

A suspension of methyl-3-(bromomethyl)benzoate (13.5 g, 58.9 mmol) and sodium cyanide (4.33 g, 88.4 mmol) in DMF (25 ml) and water (1 ml) was stirred at 75° C. for 5 hours. The reaction mixture was quenched with water (50 ml) and extracted with EtOAc (3×100 ml). The combined organics were dried with Na₂SO₄(s) and concentrated under reduced pressure. The resulting residue was purified by column chromatography utilizing an ISCO system (hexane-EtOAc) to give 7.2 g (70%) of a colourless oil. NMR (400 MHz): 7.90 (s, 1H), 7.86 (d, 1H), 7.60 (d, 1H), 7.50 (m, 1H), 4.10 (s, 2H), 3.80 (s, 3H); m/z 175.

### Method 5-6

The following compound was prepared by the procedure of Method 4, using the appropriate starting material.

| Meth | Compound | m/z | SM |
|---|---|---|---|
| 5 | Methyl 4-chloro-3-(cyanomethyl)benzoate | 210 | Method 22 |
| 6 | [4-({[tert-Butyl(diphenyl)silyl]oxy}methyl)-2- | 392 | Method 27 |
| | thienyl]acetonitrile | | |

### Method 7

### 3-(1-Cyano-1-methylethyl)benzoic acid methyl ester

A solution of 3-cyanomethyl-benzoic acid methyl ester (Method 4; 7.2 g, 41.1 mmol) in anhydrous DMSO (80 ml) was treated with sodium hydride (60%, 4.9 g, 123.3 mmol, 3 eq).Methyl iodide was then added dropwise at 0° C. The reaction mixture was stirred at 25° C. for 12 hours. The reaction mixture was then quenched with water (200 ml) and extracted with EtOAc. The combined organics were dried with Na₂SO₄(s) and concentrated under reduced pressure. The crude product was purified by column chromatography utilizing an ISCO system (hexane-EtOAc) to give 5.5 g (66%) of a colourless oil. NMR (400 MHz): 8.05 (s, 1H), 7.90 (d, 1H), 7.75 (d, 1H), 7.55 (m, 1H), 3.80 (s, 3H), 1.62 (s, 6H); m/z 203.

### Methods 8-10

The following compounds were prepared by the procedure of Method 7, using the appropriate starting material.

| Meth | Compound | m/z | SM |
|---|---|---|---|
| 8 | Methyl 4-chloro-3-(1-cyano-1- | 238 | Method 5 |
| | methylethyl)benzoate | | |
| 9 | 2-[4-({[tert- | 421 | Method 6 |
| | Butyl(diphenyl)silyl]oxy}methyl)-2- | | |
| | thienyl]-2-methylpropanenitrile | | |
| 10 | 2-Methyl-2-(2-thienyl)propanenitrile | 152 | 2-Thienyl- |
| | | | acetonitrile |

### Method 11

### 3-(1-Cyano-1-methylethyl)benzoic acid

A solution of 3-(1-cyano-1-methylethyl)benzoic acid methyl ester (Method 7; 5.5 g, 27.1 mmol) in 100 ml of THF/MeOH/H₂O (3:1:1) was treated with lithium hydroxide (1.95 g) in water (20 ml). The mixture was stirred at 25° C. for 12 hours. The volatile solvent was removed by distillation and the resulting solution was diluted with water, then acidified with 10% HCl to pH=1-3. The resulting white solid (4.83 g, 94%) was filtered, washed with water, and dried. NMR (400 MHz): 13.00 (s, 1H), 7.95 (s, 1H), 7.80 (d, 1H), 7.65 (d, 1H), 7.45 (m, 1H), 1.60 (s, 6H); m/z 189.

### Method 12

The following compound was prepared by the procedure of Method 11, using the appropriate starting material.

| Meth | Compound | m/z | SM |
|---|---|---|---|
| 12 | 4-Chloro-3-(1-cyano-1-methylethyl)benzoic | 224 | Method 8 |
| | acid | | |

### Method 13

### Methyl 3-(6-bromo-4-oxoquinazolin-3(4H)-yl)-4-methylbenzoate

A suspension of 2-amino-5-bromobenzoic acid (97 g, 0.45 mol) in anhydrous toluene (2 l) under nitrogen was treated with excess trimethylorthoformate (250 ml, 2.25 mol). A catalytic amount of acetic acid (1 ml) was added via syringe, and the heterogeneous white reaction mixture was refluxed for 3 hours. The reaction mixture was then cooled to 40° C. and methyl 3-amino-4-methylbenzoate (74 g, 0.45 mol) was added as a slurry generated by adding anhydrous toluene (1 l). The reaction mixture was refluxed for 20 hours, then cooled, diluted with EtOAc (1.5 l), and washed successively with 1 M HCl (aq) (1×600 ml), 2 M NaOH (aq) (2×400 ml), and brine (2×300 ml). The solvent was removed by reduced pressure to afford a tan solid. Recrystallization from EtOAc/hexanes provided the desired product as a white solid (105g, 167g theoretical, 63%). NMR (300 MHz): δ 8.37 (s, 1H), 8.29 (d, 1H), 8.1 (m, 3H), 7.74 (d, 1H), 7.62 (d, 1H), 3.87 (s, 3H), 2.18 (s, 3H); m/z 374.

### Method 14

### 3-(1-Cyano-1-methylethyl)-N-(4-methyl-3-nitro-phenyl)benzamide

A mixture of 4-methyl-3-nitroaniline (2.74 g, 18 mmol), 3-(1-cyano-1-methylethyl)benzoic acid (Method 11; 3.4 g, 18 mmol), EDCI (6.9 g, 36 mmol), HOBt (2.43 g, 18 mmol) and diisopropyl ethyl amine (3.48 g, 27 mmol, 1.5 eq) in DMF (30 ml) was stirred at 25° C. for 12 hours. The reaction mixture was diluted with DCM and then washed with water and brine. The organic phase was dried with Na₂SO₄(s). The solvent was removed by reduced pressure and the resulting product was purified by column chromatography utilizing an ISCO system (hexane-EtOAc) to give 4.4 g (53%). NMR (400 MHz): 10.50 (s, 1H), 8.40 (s, 1H), 7.40-7.95 (m, 6H), 3.20 (s, 3H), 1.65 (s, 6H); m/z 323.

### Method 15

### N-(3-Amino-4-methylphenyl)-3-(1-cyano-1-methylethyl)benzamide

A suspension of 3-(1-cyano-1-methylethyl)-N-(4-methyl-3-nitro-phenyl)benzamide (Method 14; 4g, 13.9 mmol) and 5% palladium on carbon in hydrazine hydrate (100 ml) and ethanol (100 ml) was heated to reflux for 3 hours, then stirred at 80° C. for 12 hours. The palladium/carbon was removed by filtration and the filtrate was concentrated. The residue was purified by column chromatography using an ISCO system (hexane-EtOAc) to give 3.7 g (91%) of an orange gum. NMR (400 MHz): 9.95 (s, 1H), 8.00 (s, 1H), 7.90 (d, 1H), 7.70 (d, 1H), 7.55 (m, 1H), 7.05 (s, 1H), 6.80-6.87 (m, 2H), 4.85 (s, 2H), 2.05 (s, 3H), 1.85 (s, 6H); m/z 293.

### Method 16

### 3-(6-Bromo-4-oxoquinazolin-3(4H)-yl)-4-methylbenzoic acid

Methyl 3-(6-bromo-4-oxoquinazolin-3(4H)-yl)-4-methylbenzoate (Method 13; 50g, 0.13 mol) was refluxed under nitrogen for 6 hours in 6 M HCl (1.2 l). The reaction mixture was cooled and the resulting product was collected by filtration. The solid was washed with water to remove trace HCl. The material was dried under vacuum and then triturated with a small amount of warm ethanol. The resulting product was collected by filtration to yield a white, finely divided solid. NMR (300 MHz): δ 8.38 (s, 1H), 8.29 (d, 1H), 8.07 (dd, 1H), 8.00 (m, 2H), 7.74 (d, 1H), 7.59 (d, 1H), 2.17 (s, 3H); m/z 359.

### Method 17

### N-Benzyloxycarbonyl-3-(6-bromo-4-oxoquinazolin-3(4H)-yl)-4-methylaniline

3-(6-Bromo-4-oxoquinazolin-3(4H)-yl)-4-methylbenzoic acid (Method 16; 8g, 22 mol) was suspended in anhydrous toluene (50 ml) and treated with triethylamine (3.3 ml, 24 mmol) under nitrogen. Diphenylphosphoryl azide (4.9 ml, 23 mmol) was added dropwise while stirring, followed by benzyl alcohol (4.6 ml, 44 mmol). The heterogeneous reaction mixture was heated to reflux for 12 hours. The reaction mixture was then cooled and water (250 ml) was added while stirring vigorously. The layers were separated and the aqueous layer extracted several times with EtOAc. The combined organic layers were washed sequentially with water (1×25 ml), saturated NaHCO₃ (aq) (1×50 ml), and brine (1×25 ml), then dried with Na₂SO₄(s). The solvents were removed under reduced pressure to afford a white solid. NMR (300 MHz): 10.0 (br s, 1H), 8.38 (s, 1H), 8.29 (d, 1H), 8.06 (dd, 1H), 7.73 (d, 1H), 7.35-7.55 (m, 8H), 2.17 (s, 3H); m/z: 465.

### Method 18

### 3-(5-Amino-2-methylphenyl)-6-bromo-3H-quinazolin-4-one

N-Benzyloxycarbonyl-3-(6-bromo-4-oxoquinazolin-3(4H)-yl)-4-methyl aniline (Method 17; 4g, 10 mmol) was suspended in 30% HBr in acetic acid and stirred vigorously at 25° C. under nitrogen atmosphere for 24 hours. Excess acetic acid was removed under reduced pressure and water (200 ml) was added while stirring vigorously. The layers were separated and the aqueous layer extracted several times with EtOAc. The combined organic layers were washed sequentially with water (1×25 ml), saturated NaHCO₃ (aq) (1×50 ml), and brine (1×25 ml), then dried with Na₂SO₄(s). The solvents were removed under reduced pressure to afford a white solid. NMR (300 MHz): 7.5-8.2 (m, 7H), 7.2 (s, 2H), 2.15 (s, 3H); m/z 330.

### Method 19

### 2-Methyl-2-(4-methylpyridin-2-yl)propanenitrile

2-Fluoro-4-methylpyridine (1.00 g, 9.00 mmol) and 2-methylpropanenitrile (2.48 g, 36 mmol) were dissolved in anhydrous toluene (30 ml). Potassium Hexamethyldisilazide (13.5 mmol) was added and the reaction was refluxed for 1 h. The reaction was then quenched with saturated aqueous NH₄Cl (50 ml) and the mixture was extracted with EtOAc (2×50 ml). The combined organic phase was dried with MgSO₄ and concentrated in vacuo to yield the crude reaction product which was purified on 40g SiO₂ hexanes-EtOAc 5:1 as eluent giving 0.870 g (60%); m/z 161.

### Method 20

### 2-(1-Cyano-1-methylethyl)isonicotinic acid

2-Methyl-2-(4-methylpyridin-2-yl)propanenitrile (Method 19; 0.870 g, 5.43 mmol) was dissolved in water (15 ml). The reaction mixture was heated to 60° C. and KMnO₄ (4.3 g, 27 mmol) was added. The reaction was heated to reflux for 2 h, and was then filtered through a bed of celite. The pH was adjusted to 4 by the careful addition of 1 N HCl and the aqueous phase was extracted with EtOAc (4×25 ml). The organic phase was dried with MgSO₄ and concentrated in vacuo to yield the crude reaction product which was purified on 40g SiO₂ using EtOAc-MeOH 10:1 as eluent giving 0.700g (68%); m/z 191.

### Method 21

The following compound was prepared by the procedure of Method 20, using the appropriate starting material.

| Meth | Compound | m/z | SM |
|---|---|---|---|
| 21 | 3-tert-Butylbenzoic acid | 179 | 1-tert-butyl-3-methylbenzene |

### Method 22

### Methyl 3-(bromomethyl)-4-chlorobenzoate

Azobis(isobutyronitrile) (500 mg) was added to methyl 4-chloro-3-methylbenzoate (2.50 g, 13.54 mmol), N-bromosuccinimide (3.00 g, 16.93 mmol) and carbon tetrachloride (50 ml). The solution was heated to 80° C. with stirring for 4 h before being cooled to 25° C. The reaction mixture was filtered through a pad of celite and the filtrate was concentrated in vacuo. The crude product was purified on 40g SiO₂ using hexanes-EtOAc 10:1 as eluent giving 2.70 g (76%); m/z 264.

### Method 23

### tert-Butyl(diphenyl)(3-thienylmethoxy)silane

Anhydrous DMF (86 ml) and imidazole (8.94 g, 131.4 mmol) were added to 3-thienylmethanol (5.0 g, 43.8 mmol). The reaction mixture was cooled to 0° C. and treated with tert-butylchlorodiphenylsilane (15.0 g, 54.7 mmol) and was allowed to stir 6 h. The reaction was warmed to 25° C. before being quenched by the addition of saturated aqueous NH₄Cl (250 ml). The resulting mixture was extracted with EtOAc (3×125 ml). The combined organic phase was washed with brine (1×100 ml), dried with MgSO₄, and concentrated in vacuo. The crude reaction product was purified on 120g SiO₂ using hexanes-EtOAc 10:1 as eluent giving 14.8 g (96%); m/z 353.

### Method 24

### 2-(5-Formyl-2-thienyl)-2-methylpropanenitrile

THF (5.8 ml) was added to 2-methyl-2-(2-thienyl)propanenitrile (Method 10; 0.260 g, 1.71 mmol) and the reaction mixture was cooled to −78° C. To the cooled reaction was added 1.26 ml of tert-butyl lithium (1.7 M solution in pentanes) drop wise via syringe. The resulting bright yellow mixture was allowed to stir for 1 h before anhydrous DMF (0.330 ml, 4.27 mmol) was added via syringe. The reaction was stirred for 6 h at −78° C. before being quenched by the addition of saturated aqueous NH₄Cl (25 ml). The resulting mixture was extracted with EtOAc (3×25 ml). The combined organic phase was washed with brine (1×50 ml), dried with MgSO₄, and concentrated in vacuo giving the title compound, 0.271 g, (88%) as a colourless oil; m/z 180.

### Method 25

The following compound was prepared by the procedure of Method 24, using the appropriate starting material.

| Meth | Compound | m/z | SM |
|---|---|---|---|
| 25 | 4-({[tert- | 381 | Method 23 |
| | Butyl(diphenyl)silyl]oxy}methyl)thiophene-2- | | |
| | carbaldehyde | | |

### Method 26

### [4-({[tert-Butyl(diphenyl)silyl]oxy}methyl)-2-thienyl]methanol

4-({[tert-Butyl(diphenyl)silyl]oxy}methyl)thiophene-2-carbaldehyde (Method 25; 3.99 g, 10.48 mmol) was dissolved in methanol (50 ml). NaBH₄ (0.792 g, 20.96 mmol) was added in one portion. After 1 h, the reaction was carefully quenched with a solution of saturated NH₄Cl (250 ml). The resulting mixture was extracted with EtOAc (3×125 ml). The combined organic phase was washed with brine (1×250 ml), dried with MgSO₄, and conc. in vacuo giving the crude reaction product which purified on 120g SiO₂ using hexanes/EtOAc 5:2 as eluent giving 3.99 g of the title compound as a colourless oil (98%); m/z 384.

### Method 27

### {[5-(Bromomethyl)-3-thienyl]methoxy}(tert-butyl)diphenylsilane

Anhydrous THF (45 ml) was added to [4-({[tert-butyl(diphenyl)silyl]oxy}methyl)-2-thienyl]methanol (Method 26; 4.2 g, 10.98 mmol). Phosphorous tribromide (3.56 g, 13.17 mmol) was added dropwise via syringe and the reaction was allowed to stir for 1 h at 25° C. before being quenched by saturated aqueous NaHCO₃ (250 ml). The reaction mixture was extracted with EtOAc (2×250 ml) and the combined organic phase was dried with MgSO₄ and concentrated in vacuo to yield the crude reaction product which was purified on 120g SiO₂ hexanes-EtOAc 10:1 as eluent giving 3.70 g (76%); m/z 447.

### Method 28

### 2-[4-(Hydroxymethyl)-2-thienyl]-2-methylpropanenitrile

Anhydrous THF (25 ml) was added to 2-[4-({[tert-butyl(diphenyl)silyl]oxy}methyl)-2-thienyl]-2-methylpropanenitrile (Method 9; 0.880 g, 2.10 mmol). A 1M solution of tetrabutylammonium fluoride in THF (5.25 mmol) was added dropwise via syringe and the reaction was allowed to stir for 12 h. at 25° C. before being quenched saturated aqueous NH₄Cl (50 ml). The reaction mixture was extracted with EtOAc (2×50 ml) and the combined organic phase was dried with MgSO₄ and concentrated in vacuo to yield the crude reaction product which was purified on 40g SiO₂ hexanes-EtOAc 2:1 as eluent giving 0.270 g (71%); m/z 182.

### Method 29

### 2-(4-Formyl-2-thienyl)-2-methylpropanenitrile

To DMSO (0.277 g, 3.55 mmol) was added DCM (10 ml). The reaction was cooled to −78° C. and oxalyl chloride (0.225 g, 1.78 mmol) was added dropwise via syringe and the reaction was allowed to stir for 30 min at this temperature. A 1 M solution of 2-[4-(hydroxymethyl)-2-thienyl]-2-methylpropanenitrile (Method 28; 0.270 g, 1.48 mmol) in DCM was then added dropwise via syringe and the reaction was allowed to stir for 30 min. at this temperature. Triethylamine (0.718 g, 7.40 mmol) was then added and the reaction was allowed to warm to 25° C. with stirring over 1 h before being quenched with saturated aqueous NaHCO₃ (250 ml). The reaction mixture was then extracted with EtOAc (2×50 ml) and the combined organic phase was dried with MgSO₄ and concentrated in vacuo to yield the crude reaction product which was purified on 40g SiO₂ hexanes-EtOAc 10:1 as eluent giving 0.262 g (99%); m/z 180.

### Method 30

### 5-(1-Cyano-1-methylethyl)thiophene-2-carboxylic acid

To 2-(5-formyl-2-thienyl)-2-methylpropanenitrile (Method 24; 0.271 g, 1.51 mmol) was added 7.5 ml of tertiary butyl alcohol and 4.5 ml of 2-methyl-2-butene. The reaction mixture was treated dropwise with an aqueous pre-mixed solution of NaClO₂ (1.22 g, 13.60 mmol) and NaH₂PO₄ (1.45 g, 10.57 mmol) in H₂O (7 ml). The reaction mixture was stirred for 30 min. at 25° C. before the volatiles were removed under reduced pressure. The resulting crude product was washed with saturated aqueous NaHCO₃ (1×50 ml) and extracted with EtOAc (3×25 ml). The combined organic phase was washed with brine (1×50 ml), dried with MgSO₄, and conc. in vacuo giving 0.265 g (90%) as a white solid; m/z 196.

### Method 31

The following compound was prepared by the procedure of Method 30, using the appropriate starting material.

| Meth | Compound | m/z | SM |
|---|---|---|---|
| 31 | 5-(1-Cyano-1-methylethyl)thiophene-3- | 196 | Method 29 |
| | carboxylic acid | | |

### Method 32

### 3-(Morpholin-4-ylsulfonyl)benzoic acid

A solution of 3-(chlorosulfonyl)benzoic acid (1.00 g, 4.53 mmol) in DCM (10 ml) was treated with morpholine (3.95 ml, 45.3 mmol, 10 equiv). After 30 min, the reaction was quenched with 10% HCl and extracted with EtOAc. The organics were washed with NaCl₍ₛₐₜ₎ and then dried with Na₂SO₄₍ₛ₎. The organics were then removed under reduced pressure to give 1.10 g, 89%; m/z 272.

### Methods 33-34

The following compounds were prepared by the procedure of Method 32, using the appropriate starting material.

| Meth | Compound | m/z | SM |
|---|---|---|---|
| 33 | 3-(Azetidin-1-ylsulfonyl)-benzoic acid | 241 | Azetidine |
| 34 | 3-[(Cyclopropylamino)sulfonyl]benzoic | 241 | Cyclopropyl- |
| | acid | | amine |

### Method 35

### tert-Butyl (4-methyl-3-nitrophenyl)carbamate

A solution of 4-methyl-3-nitroaniline (10.0 g, 0.066 mol) was dissolved in THF (25 ml) at 65° C. Di-tert-butyl dicarbonate (17.2 g, 0.079 mol, 1.2 equiv) in THF (20 ml) was added dropwise over 30 min. The mixture was then refluxed under nitrogen for 12 h. The reaction was cooled to 25° C. and the solvent was removed under reduced pressure to give a brown oil. The oil was dissolved in hexane-EtOAc (4:1), (200 ml) and 30g of silica gel was added to the solution. The solution was stirred for 5 min and the silica was removed by filtration. The silica was then repeatedly washed with hexane-EtOAc (4:1) until no further product was detected. The solvents were combined and concentrated under reduced pressure. The resulting yellow solid was washed with hexane and air dried to give 14.2 g of the desired product (85%). NMR (300 MHz): 8.07 (s, 1H), 7.53 (d, 1H), 7.26-7.30 (m, 1H), 6.66 (s, 1H), 2.55 (s, 3H), 1.55 (s, 9H).

### Method 36

### tert-Butyl (3-amino-4-methylphenyl)carbamate

A solution of tert-butyl (4-methyl-3-nitrophenyl)carbamate (Method 35; 10.0 g, 39.6 mmol) was dissolved in EtOH (220 ml). The solution was treated with 10% Pd/C (650 mg) and placed on a Parr Hydrogenator at 50 psi of hydrogen for 12 h. The resulting solution was filtered through celite and the solvent was removed under reduced pressure to give 8.68 g (98%). NMR (300 MHz): 6.86-6.98 (m, 2H), 6.48 (d, 1H), 6.36 (s, 1H), 3.59 (s, 2H), 2.09 (s, 3H), 1.42-1.50 (m, 9H).

### Method 37

### 8-Methoxy-4H-3,1-benzoxazin-4-one

A solution of 2-amino-3-methoxybenzoic acid (10.0 g, 59.8 mmol), triethylorthoformate (45 ml, 270 mmol) and acetic acid (1 ml) were refluxed in toluene (100 ml) for 12 h utilizing a Dean-Stark trap to remove water. The solvent was then removed under reduced pressure. The remaining solid was dissolved in DCM and washed with water. The solution was dried over Na₂SO₄ and the solvents were removed under reduced pressure. NMR (300 MHz): 7.77-7.89 (m, 2H), 7.51 (t, 1H), 7.31 (d, 1H), 4.01 (s, 3H).

### Method 38

### tert-Butyl [3-(8-methoxy-4-oxoquinazolin-3(4H)-yl)-4-methylphenyl]carbamate

A solution of 8-methoxy-4H-3,1-benzoxazin-4-one (Method 37; 200 mg, 1.13 mmol) and tert-butyl (3-amino-4-methylphenyl)carbamate (Method 36; 138 mg, 1.13 mmole) were refluxed in toluene (10 ml) for 12 h. The solvent was removed under reduced pressure and the residue was purified by column chromatography (silica gel) using EtOAc-DCM (3:1) to yield 90 mg of a white solid (21%). NMR (300 MHz): 9.56 (s, 1H), 8.19 (s, 1H), 7.73 (d, 1H), 7.53 (t, 2H), 7.38-7.48 (m, 2H), 7.25-7.33 (m, 1H), 3.89-3.95 (s, 3H), 1.98 (s, 3H), 1.45 (s, 9H).

### Method 39

### 3-(5-Amino-2-methylphenyl)-8-methoxyquinazolin-4(3H)-one

A solution of tert-butyl [3-(8-methoxy-4-oxoquinazolin-3(4H)-yl)-4-methylphenyl]carbamate (Method 38; 1.00 g, 2.62 mmol) in dioxane (25 ml) was treated with HCl (4 M in dioxane, 25 ml). The mixture was stirred at 25° C. for 12 h. Approximately 50% of the solvent was removed under reduced pressure and the remaining solution was dissolved in 15 ml of water. The pH of the solution was adjusted to 12 by the addition of NH₄OH. The mixture was then extracted three times with EtOAc. The combined solvents were dried over Na₂SO₄ and concentrated under reduced pressure to give 0.4 g (54%) as a pale yellow foam. NMR (300 MHz): 7.91-8.02 (m, 2H), 7.48 (t, 1H), 7.22-7.28 (m, 2H), 7.14 (d, 1H), 6.69-6.75 (m, 1H), 6.56 (s, 1H), 6.04-6.16 (m, 1H), 4.05 (s, 3H), 2.04 (s, 3H).

### Method 40

### 3-(5-Amino-2-methylphenyl)-6-(4-methyl-1,4-diazepan-1-yl)quinazolin-4(3H)-one

3-(5-Amino-2-methylphenyl)-6-(4-methyl-1,4-diazepan-1-yl)quinazolin-4(3H)-one was prepared by reacting 2-amino-N-(5-amino-2-methylphenyl)-5-(4-methyl-1,4-diazepan-1-yl)benzamide (Method 42) with triethylorthoformate.

### Method 41

The following compound was prepared by the procedure of Method 40, using the appropriate starting material.

| Meth | Compound | SM |
|---|---|---|
| 41 | 3-(5-Amino-2-methylphenyl)-6-(4-methylpiperazin- | Method 43 |
| | 1-yl)quinazolin-4(3H)-one | |

### Method 42

### 2-Amino-N-(5-amino-2-methylphenyl)-5-(4-methyl-1,4-diazepan-1-yl)benzamide

2-Amino-N-(5-amino-2-methylphenyl)-5-(4-methyl-1,4-diazepan-1-yl)benzamide was prepared by a reduction of 5-(4-methyl-1,4-diazepan-1-yl)-N-(2-methyl-5-nitrophenyl)-2-nitrobenzamide (Method 44) with H₂ and Pd/C.

### Method 43

The following compound was prepared by the procedure of Method 42, using the appropriate starting material.

| Meth | Compound | SM |
|---|---|---|
| 43 | 2-Amino-N-(5-amino-2-methylphenyl)-5-(4- | Method 45 |
| | methylpiperazin-1-yl)benzamide | |

### Method 44

### 5-(4-Methyl-1,4-diazepan-1-yl)-N-(2-methyl-5-nitrophenyl)-2-nitrobenzamide

5-(4-Methyl-1,4-diazepan-1-yl)-N-(2-methyl-5-nitrophenyl)-2-nitrobenzamide was prepared by an amide bond coupling of 5-(4-methyl-1,4-diazepan-1-yl)-2-nitrobenzoic acid (Method 46) with 2-methyl-5-nitroaniline.

### Method 45

The following compound was prepared by the procedure of Method 44, using the appropriate starting material.

| Meth | Compound | SM |
|---|---|---|
| 45 | N-(2-Methyl-5-nitrophenyl)-5-(4-methylpiperazin- | Method 47 |
| | 1-yl)-2-nitrobenzamide | |

### Method 46

### 5-(4-Methyl-1,4-diazepan-1-yl)-2-nitrobenzoic acid

5-(4-Methyl-1,4-diazepan-1-yl)-2-nitrobenzoic acid was prepared by reacting 5-fluoro-2-nitrobenzoic acid with 1-methyl-1,4-diazepane.

### Method 47

The following compound was prepared by the procedure of Method 47, using the appropriate amine

| Meth | Compound | SM |
|---|---|---|
| 47 | 5-(4-Methylpiperazin-1-yl)-2-nitrobenzoic | 1-Methylpiperazine |
| | acid | |

1. A compound of formula (I): 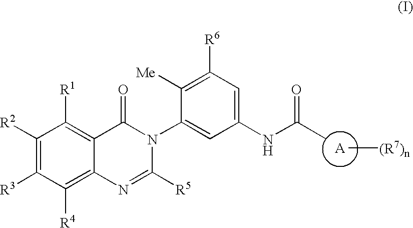
wherein:
Ring A is a 5 or 6 membered carbocyclyl or a 5 or 6 membered heterocyclyl; wherein if said heterocyclyl contains an —NH— moiety that nitrogen may be optionally substituted by a group selected from R²⁰;
R¹, R², R³, R⁴ and R⁵ are independently selected from hydrogen, halo, nitro, cyano, hydroxy, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, N′—(C₁₋₆alkyl)ureido, N′,N′—(C₁₋₆alkyl)₂ureido, C₁₋₆alkylS(O)₂ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁶— or heterocyclyl-R¹⁶—; wherein at least one R¹, R², R³, R⁴ and R⁵ is not hydrogen; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹;
R⁶ is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁷—; wherein R⁶ may be optionally substituted on carbon by one or more R¹⁰; and wherein if said heterocyclyl contains an —NH-moiety that nitrogen may be optionally substituted by a group selected from R¹¹;
R⁷ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁸— or heterocyclyl-R¹⁸—; wherein R⁷ may be optionally substituted on carbon by one or more R¹²; and wherein if said heterocyclyl contains an —NH— moiety that nitrogen may be optionally substituted by a group selected from R¹³;
n is selected from 1-4; wherein the values of R⁷ may be the same or different;
R⁸, R¹⁰ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁹— or heterocyclyl-R¹⁹—; wherein R⁸, R¹⁰ and R¹² independently of each other may be optionally substituted on carbon by one or more R¹⁴; and wherein if said heterocyclyl contains an —NH— moiety that nitrogen may be optionally substituted by a group selected from R¹⁵;
R¹⁶, R¹⁷ and R¹⁹ are independently selected from a direct bond, —O—, —N(R²¹)—, —C(O)—, —N(R²¹)C(O)—, —C(O)N(R²¹)—, —S(O)ₛ—, —SO₂N(R²¹)— or —N(R²¹)SO₂—; wherein R²¹ is hydrogen or C₁₋₆alkyl and s is 0-2;
R¹⁸ is —N(R²²)—-C(O)— —N(R²²)C(O)—, —C(O)N(R²²)—, —S(O)ₛ—, —SO₂N(R²²)— or —N(R²²)SO₂—; wherein R²² is hydrogen or C₁₋₆alkyl and s is 0-2;
R⁹, R¹¹, R¹³, R¹⁵ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R¹⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof; with the proviso that said compound is not: 2-chloro-N-{4-methyl-3-[6-(4-methylpiperazin-1-yl)-4-oxoquinazolin-3(4H)-yl]phenyl}isonicotinamide; 3,5-difluoro-N-{4-methyl-3-[6-(4-methylpiperazin-1-yl)-4-oxoquinazolin-3(4H)-yl]phenyl}benzamide; 3-(acetylamino)-N-{4-methyl-3-[6-(4-methylpiperazin-1-yl)-4-oxoquinazolin-3(4H)-yl]phenyl}benzamide; 3-fluoro-N-{4-methyl-3-[6-(4-methylpiperazin-1-yl)-4-oxoquinazolin-3(4H)-yl]phenyl}-4-(trifluoromethyl)benzamide; 2-methoxy-N-{4-methyl-3-[6-(4-methylpiperazin-1-yl)-4-oxoquinazolin-3(4H)-yl]phenyl}benzamide; 3-ethoxy-N-{4-methyl-3-[6-(4-methylpiperazin-1-yl)-4-oxoquinazolin-3(4H)-yl]phenyl}benzamide; N-{4-methyl-3-[6-(4-methylpiperazin-1-yl)-4-oxoquinazolin-3(4H)-yl]phenyl}-3-(1,1,2,2-tetrafluoroethoxy)benzamide; 3-chloro-N-{4-methyl-3-[6-(4-methyl-1,4-diazepan-1-yl)-4-oxoquinazolin-3(4H)-yl]phenyl}isonicotinamide; 3,5-difluoro-N-{4-methyl-3-[6-(4-methyl-1,4-diazepan-1-yl)-4-oxoquinazolin-3(4H)-yl]phenyl}benzamide; 4-methoxy-N-[4-methyl-3-(2-methyl-4-oxoquinazolin-3(4H)-yl)phenyl]benzamide; or 4-methyl-N-[4-methyl-3-(2-methyl-4-oxoquinazolin-3(4H)-yl)phenyl]benzamide.

2. A compound of formula (I), or a pharmaceutically acceptable salt thereof, as claimed in claim 1 wherein Ring A is phenyl, pyridyl, thienyl or pyrazolyl; wherein said pyrazolyl may be optionally substituted on nitrogen by a group selected from R²⁰; wherein R²⁰ is C₁₋₆alkyl.

3. A compound of formula (I), or a pharmaceutically acceptable salt thereof, as claimed in claim 1 wherein:
R¹, R², R³, R⁴ and R⁵ are independently selected from hydrogen, halo, hydroxy, amino, carboxy, carbamoyl, C₁₋₆alkyl, C₂₋₆alkynyl, C₁₋₆alkoxy, N—(C₁₋₆alkyl)amino, N—(C₁₋₆alkyl)carbamoyl, N′—(C₁₋₆alkyl)ureido, C₁₋₆alkylsulphonylamino, carbocyclyl-R¹⁶— or heterocyclyl-R¹⁶—; wherein at least one R¹, R², R³, R⁴ and R⁵ is not hydrogen; wherein R¹, R², R³, R⁴ and R⁵ independently of each other may be optionally substituted on carbon by one or more R⁸; and wherein if said heterocyclyl contains an —NH— moiety that nitrogen may be optionally substituted by a group selected from R⁹;
R⁸ is selected from hydroxy, amino, C₁₋₆alkyl, C₁₋₆alkoxy, N—(C₁₋₆alkyl)amino, N,N—(C₁₋₆alkyl)₂amino, C₁₋₆alkanoylamino, N—(C₁₋₆alkyl)carbamoyl or heterocyclyl-R¹⁹—; wherein R⁸, R¹⁰ and R¹² independently of each other may be optionally substituted on carbon by one or more R¹⁴; and wherein if said heterocyclyl contains an —NH— moiety that nitrogen may be optionally substituted by a group selected from R¹⁵;
R¹⁶ and R¹⁹ are independently selected from a direct bond, —N(R²¹)—, —N(R²¹)C(O)— or —C(O)N(R²¹)—; wherein R²¹ is hydrogen;
R⁹ and R¹⁵ are independently selected from C₁₋₆alkyl and C₁₋₆alkoxycarbonyl; and
R¹⁴ is methoxy.

4. A compound of formula (I), or a pharmaceutically acceptable salt thereof, as claimed in claim 1 wherein R⁶ is hydrogen.

5. A compound of formula (I), or a pharmaceutically acceptable salt thereof, as claimed in claim 1 wherein:
R⁷ is a substituent on carbon and is selected from halo, C₁₋₆alkyl, C₁₋₆alkoxy, C₁₋₆alkylS(O)ₐ wherein a is 2, C₁₋₆alkylsulphonylamino, carbocyclyl-R¹⁸— or heterocyclyl-R¹⁸—; wherein R⁷ may be optionally substituted on carbon by one or more R¹²;
R¹² is selected from halo or cyano;
R¹⁸ is —S(O)ₛ— or —N(R²²)SO₂—; wherein R²² is hydrogen and s is 0-2.

6. A compound of formula (I), or a pharmaceutically acceptable salt thereof, as claimed in claim 1 wherein n is selected from 1 or 2; wherein the values of R⁷ may be the same or different.

7. A compound of formula (I): 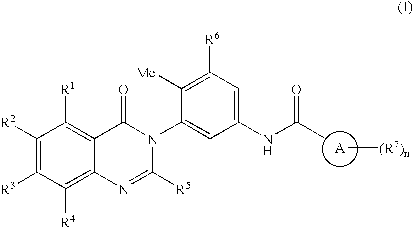
wherein:
Ring A is phenyl, 1-t-butylpyrazol-5-yl, 1-methylpyrazol-5-yl, pyrid-2-yl, pyrid-3-yl, pyrid-4-yl, thien-2-yl and thien-3-yl;
R¹, R², R³, R⁴ and R⁵ are independently selected from hydrogen, chloro, bromo, hydroxy, amino, carboxy, carbamoyl, methyl, 3-dimethylaminopropyl, 3-methylaminopropyl, 3-acetylaminopropyl, methoxy, N-methylcarbamoyl, N-(2-ethoxyethyl)carbamoyl, N-(2-dimethylaminoethyl)carbamoyl, N-[2-(imidazol-4-yl)ethyl]carbamoyl, 3-(amino)prop-1-yn-1-yl, 3-(acetylamino)prop-1-yn-1-yl, 3-(methylamino)prop-1-yn-1-yl, 3-(dimethylamino)prop-1-yn-1-yl, N′-methylureido, mesylamino, 2-(dimethylamino)ethoxy, 2-(diethylamino)ethoxy, 3-(dimethylamino)propoxy, 2-morpholinoethoxy, 3-morpholinopropoxy, 2-(piperidin-1-yl)ethoxy, 2-(pyrrolidino)ethoxy, oxiranylmethoxy, 3-(1-methylpiperazin-4-yl)propoxy, 2-(pyrrolidin-1-yl)ethylamino, 2-hydroxypropylamino, 2-(piperidin-1-yl)ethylamino, 3-(dimethylamino)propylamino, 2-(pyrid-2-yl)ethylamino, 1-(t-butoxycarbonyl)azetidin-3-ylamino, azetidin-3-ylamino, (N-methylcarbamoyl)methylamino, tetrahydrofuran-2-ylmethylamino, 2-methoxyethylamino, 3-(piperidin-1-yl)propylamino, cyclopropylaminocarbonyl, cyclopropylcarbonylamino, pyrazol-3-ylaminocarbonyl, 1,3,4-thiadiazol-2-ylaminocarbonyl, 5-methyl-1,3,4-thiadiazol-2-ylaminocarbonyl, 1,2,3-thiadiazol-4-ylcarbonylamino, 1-ethylpiperazin-4-yl, 1-isopropylpiperazin-4-yl, morpholino, azetidin-3-ylamino, pyrid-3-yl, pyrrol-2-yl, pyrazol-4-yl, pyrimidin-5-yl, 3-dimethylaminopyrrolidin-1-yl, 4-(piperidin-1-yl)piperidin-1-yl, (2S)-2-(methoxymethyl)pyrrolidin-1-yl and 1-methylpiperazin-4-yl;
R⁶ is hydrogen;
R⁷ is selected from fluoro, chloro, methyl, t-butyl, methoxy, mesyl, cyclopropylaminosulphonyl, azetidin-1-ylsulphonyl, morpholinosulphonyl, mesylamino, trifluoromethyl or 1-cyano-1-methylethyl;
n is selected from 1 or 2; wherein the values of R⁷ may be the same or different;
or a pharmaceutically acceptable salt thereof;
with the proviso that said compound is not: 2-chloro-N-{4-methyl-3-[6-(4-methylpiperazin-1-yl)-4-oxoquinazolin-3(4H)-yl]phenyl}isonicotinamide; 3,5-difluoro-N-{4-methyl-3-[6-(4-methylpiperazin-1-yl)-4-oxoquinazolin-3(4H)-yl]phenyl}benzamide; 2-methoxy-N-{4-methyl-3-[6-(4-methylpiperazin-1-yl)-4-oxoquinazolin-3(4H)-yl]phenyl}benzamide; 4-methoxy-N-[4-methyl-3-(2-methyl-4-oxoquinazolin-3(4H)-yl)phenyl]benzamide; or 4-methyl-N-[4-methyl-3-(2-methyl-4-oxoquinazolin-3(4H)-yl)phenyl]benzamide.

8. A compound of formula (I): 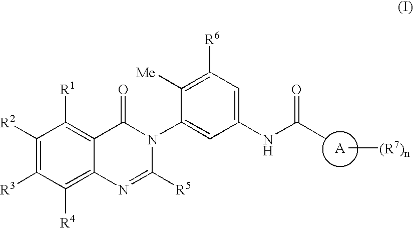
selected from:
3-(1-cyano-1-methylethyl)-N-{3-[6-(4-ethylpiperazin-1-yl)-4-oxo-4H-quinazolin-3-yl]-4-methylphenyl}benzamide;
3-(1-cyano-1-methylethyl)-N-[4-methyl-3-(4-oxo-7-pyrimidin-5-yl-4H-quinazolin-3-yl)phenyl]-benzamide;
3-(1-cyano-1-methylethyl)-N-{4-methyl-3-[4-oxo-7-(1H-pyrazol-4-yl)-4H-quinazolin-3-yl]-phenyl}benzamide;
3-(1-cyano-1-methylethyl)-N-[3-(8-methoxy-4-oxo-4H-quinazolin-3-yl)-4-methylphenyl]-benzamide;
3-(1-cyano-1-methylethyl)-N-{4-methyl-3-[4-oxo-8-(1H-pyrazol-4-yl)-4H-quinazolin-3-yl]-phenyl}-benzamide;
N-{3-[6-(1,4′-bipiperidin-1′-yl)-4-oxoquinazolin-3(4H)-yl]-4-methylphenyl}-3-(1-cyano-1-methylethyl)benzamide;
3-(1-cyano-1-methylethyl)-N-{3-[6-(4-isopropylpiperazin-1-yl)-4-oxoquinazolin-3(4H)-yl]-4-methyl phenyl}benzamide;
3-(5-{[3-(1-cyano-1-methylethyl)benzoyl]amino}-2-methylphenyl)-N-(2-methoxyethyl)-4-oxo-3,4-dihydroquinazoline-8-carboxamide;
3-(5-{[3-(1-cyano-1-methylethyl)benzoyl]amino}-2-methylphenyl)-4-oxo-N-1,3,4-thiadiazol-2-yl-3,4-dihydroquinazoline-8-carboxamide;
3-(cyano-dimethyl-methyl)-N-[4-methyl-3-(4-oxo-7-pyridin-3-yl-4H-quinazolin-3-yl)-phenyl]-benzamide;
3-(1-cyano-1-methylethyl)-N-{4-methyl-3-[6-(4-methyl-1,4-diazepan-1-yl)-4-oxoquinazolin-3(4H)-yl]phenyl}benzamide; and
3-(1-cyano-1-methylethyl)-N-{4-methyl-3-[6-(4-methylpiperazin-1-yl)-4-oxoquinazolin-3(4H)-yl]phenyl}benzamide;
or a pharmaceutically acceptable salt thereof.

9. A process for preparing a compound of formula (I) or a pharmaceutically acceptable salt thereof, as claimed in claim 1, which process, wherein variable are, unless otherwise specified, as defined in claim 1, comprises of:
Process a) reacting an amine of the formula (II): 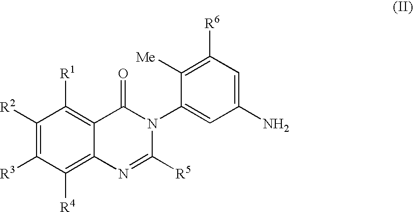
with an acid of formula (III): 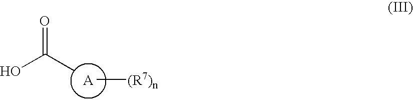
or an activated acid derivate thereof; or
Process b) reacting an amine of formula (VI): 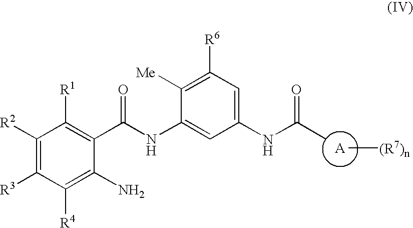
with an compound of formula (V):
(R^{a}O)₃CR⁵ (V)
wherein R^{a} is methyl or ethyl; or
Process c) reaction of an amine of formula (VI): 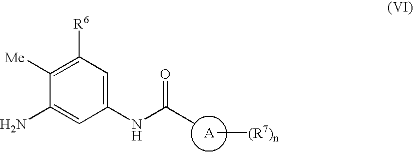
with a benzo[d][1,3]oxazin-4-one of the formula (VII): 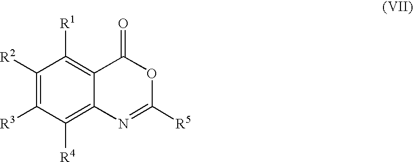
and thereafter if necessary:
i) converting a compound of the formula (I) into another compound of the formula (I);
ii) removing any protecting groups;
iii) forming a pharmaceutically acceptable salt.

10. A pharmaceutical composition which comprises a compound of the formula (I), or a pharmaceutically acceptable salt thereof, as claimed in claim 1, in association with a pharmaceutically-acceptable diluent or carrier.

11-14. (canceled)

15. A method for producing a B-Raf inhibitory effect in a warm-blooded animal, such as man, in need of such treatment which comprises administering to said animal an effective amount of a compound of formula (I), or a pharmaceutically acceptable salt thereof, as claimed in claim 1.

16. A method for producing an anti-cancer effect in a warm-blooded animal, such as man, in need of such treatment which comprises administering to said animal an effective amount of a compound of formula (I), or a pharmaceutically acceptable salt thereof, as claimed in claim 1.

17. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof, as claimed in claim 1.

18-20. (canceled)
